(19) United States
(12) Patent Application Publication
(10) Pub. No.: US 2024/0261444 A1
(43) Pub. Date: Aug. 8, 2024

(54) RIGIDIFIED MACROCYCLES, COMPLEXES WITH RADIONUCLIDES, AND USE IN TARGETED RADIOTHERAPY OF CANCER

(71) Applicant: Cornell University, Ithaca, NY (US)

(72) Inventors: Karthika J. Kadassery, Ithaca, NY (US); Justin Wilson, Ithaca, NY (US)

(73) Assignee: Cornell University, Ithaca, NY (US)

(21) Appl. No.: 18/562,744

(22) PCT Filed: May 26, 2022
(86) PCT No.: PCT/US2022/031132

§ 371 Date: Nov. 20, 2023

Related U.S. Application Data
(60) Provisional application No. 63/193,428, filed on May 26, 2021.

(51) Int. Cl. A61K 51/04; A61K 51/10; C07B 59/00

(52) U.S. Cl. CPC A61K 51/0482; A61K 51/1096; C07B 59/002

(57) ABSTRACT

The present technology provides compounds, as well as compositions including such compounds, useful in targeted radiotherapy of cancer and/or mammalian tissue overexpressing e.g., a glypican-3 (GPC3) receptor and/or prostate specific membrane antigen, where the compounds are represented by the Formulas (I) or a pharmaceutically acceptable salt and/or solvate thereof, (II) or a pharmaceutically acceptable salt and/or solvate thereof, (III) or a pharmaceutically acceptable salt and/or solvate thereof, wherein M¹ is independently at each occurrence a radionuclide. Equivalents of such compounds are also disclosed.

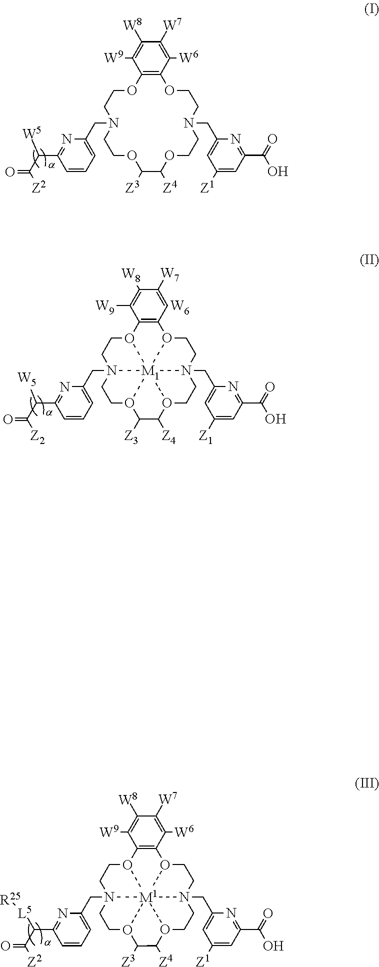

### CROSS-REFERENCE TO RELATED APPLICATIONS

This application claims the benefit of and priority to U.S. Provisional Appl. No. 63/193,428 filed on May 26, 2021, which is incorporated herein by reference in its entirety for any and all purposes.

### U.S. GOVERNMENT RIGHTS

This invention was made with government support under R21EB027282 and R01EB02925 awarded by the National Institutes of Health. The government has certain rights in the invention.

### FIELD

The present technology generally relates to macrocycles and macrocyclic complexes of radionuclides, compositions including such compounds and complexes, and methods of use.

### SUMMARY

In an aspect, a compound of Formula (I) is provided:

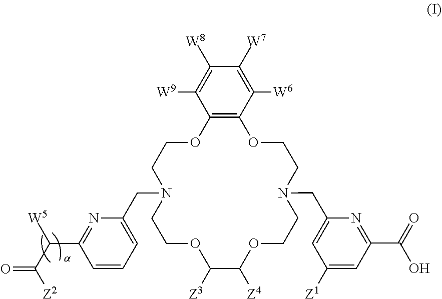

or a pharmaceutically acceptable salt and/or solvate thereof, wherein
Z¹ is H or —X¹—W¹;
Z² is OH or NH—W²;
Z³ is H or W³, and Z⁴ is H or W⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W¹, W², W⁶, W⁷, W⁸, and W⁹ are each independently H, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)_{y}—R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group;
W³, W⁴, W⁵, and W¹⁰ are each independently OH, NH₂, SH, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)yₓ-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group; and
R′ is independently at each occurrence H, halo, —N₃, C₁-C₆ alkyl, C₃-C₆ cycloalkyl, C₂-C₆ alkenyl, C₅-C₅ cycloalkenyl, C₂-C₆ alkynyl, C₅-C₁₀ cycloalkynyl, C₅-C₆ aryl, heterocyclyl, or heteroaryl.

In a related aspect, a compound of Formula (II) is provided

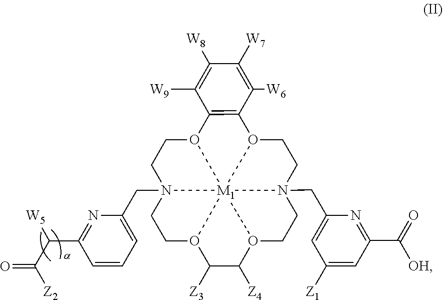

or a pharmaceutically acceptable salt and/or solvate thereof, wherein
M¹ is a radionuclide;
Z¹ is H or —X¹—W¹;
Z² is OH or NH—W²;
Z³ is H or W³, and Z⁴ is H or W⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W¹, W², W⁶, W⁷, W⁸, and W⁹ are each independently H, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)_{y}—R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group;
W³, W⁴, W⁵, and W¹⁰ are each independently OH, NH₂, SH, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)yₓ-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group; and
R′ is independently at each occurrence H, halo, —N₃, C₁-C₆ alkyl, C₃-C₆ cycloalkyl, C₂-C₆ alkenyl, C₅-C₅ cycloalkenyl, C₂-C₆ alkynyl, C₅-C₁₀ cycloalkynyl, C₅-C₆ aryl, heterocyclyl, or heteroaryl.

In a further related aspect , the present technology provides a compound (e.g., a “targeting compound”) useful in targeted radiotherapy of cancer and/or mammalian tissue overexpressing e.g., a glypican-3 (GPC3) receptor and/or PSMA, where the compound is of Formula (III)

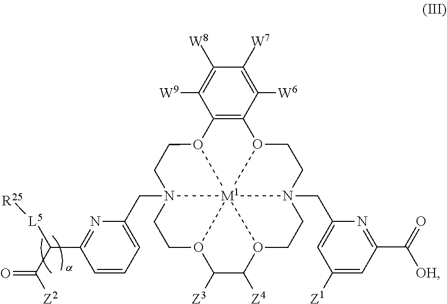

or a pharmaceutically acceptable salt and/or solvate thereof, wherein
M¹ is a radionuclide;
Z¹ is H or —X¹-L¹-R²¹;
Z² is OH or NH-L²-R²²;
Z³ is H or -L³-R²³, and Z⁴ is H or -L⁴-R²⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W⁶, W⁷, W⁸, W⁹, and W¹⁰ are each independently H or -L⁷-R²⁷;
L¹, L², L³, L⁴, L⁵, and L⁷ are each independently at each occurrence a bond or a linker group; and
R²¹, R²², R²³, R²⁴, R²⁵, and R²⁷ are each independently comprises an antibody, antibody fragment (e.g., an antigen-binding fragment), a binding moiety, a binding peptide, a binding polypeptide (such as a selective targeting oligopeptide containing up to 50 amino acids), a binding protein, an enzyme, a nucleobase-containing moiety (such as an oligonucleotide, DNA or RNA vector, or aptamer), or a lectin.

In a further related aspect, a modified antibody, modified antibody fragment, or modified binding peptide comprising a linkage arising from conjugation of a compound of Formula (I) or pharmaceutically acceptable salt and/or solvate thereof, with an antibody, antibody fragment, or binding peptide. In a related aspect, a modified antibody, modified antibody fragment, or modified binding peptide is provided that includes a linkage arising from conjugation of a compound of Formula (II) or a pharmaceutically acceptable salt and/or solvate thereof, with an antibody, antibody fragment, or binding peptide.

In any embodiment and/or aspect disclosed herein (for simplicity's sake, hereinafter recited as “in any embodiment disclosed herein” or the like), it may be that the antibody includes Codrituzumab (GC33), belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, or Etaracizumab. In any embodiment disclosed herein, it may be that the antibody fragment includes an antigen-binding fragment of Codrituzumab (GC33), belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, or Etaracizumab. In any embodiment disclosed herein, it may be that the binding peptide includes a prostate specific membrane antigen (“PSMA”) binding peptide, a somatostatin receptor agonist, a bombesin receptor agonist, a seprase binding compound, or a binding fragment thereof. In any embodiment disclosed herein, it may be that the binding peptide includes Codrituzumab (GC33), or a binding fragment thereof.

In another aspect, the present technology also provides compositions (e.g., pharmaceutical compositions) and medicaments comprising any of one of the embodiments of the compounds of Formulas I, II, or III (or a pharmaceutically acceptable salt and/or solvate thereof) disclosed herein and a pharmaceutically acceptable carrier or one or more excipients or fillers. In a similar aspect, the present technology also provides compositions (e.g., pharmaceutical compositions) and medicaments comprising any of one of the embodiments of the modified antibody, modified antibody fragment, or modified binding peptide of the present technology disclosed herein and a pharmaceutically acceptable carrier or one or more excipients or fillers.

In an aspect, a method of treating a subject is provided, wherein the method includes administering a targeting compound of the present technology to the subject or administering a modified antibody, modified antibody fragment, or modified binding peptide of the present technology to the subject. In any embodiment disclosed herein, it may be that the subject suffers from cancer and/or a mammalian tissue overexpressing glypican-3 (GPC3) receptor and/or a mammalian tissue overexpressing prostate specific membrane antigen (“PSMA”).

In an aspect, a compound is provided that includes a first domain having a blood-protein binding moiety with low specific affinity for the blood-protein, a second domain having a tumor targeting moiety with high affinity for a tumor antigen, and a third domain having a chelator.

### BRIEF DESCRIPTION OF THE DRAWINGS

FIGS. 1A-1B shows x-ray crystal structures of [La(BZmacropa)(H₂O)](PF6) (FIG. 1A), and [La(BZ2macropa)(H₂O)](PF6) (FIG. 1). Thermal ellipsoids are drawn at the 50% probability level. Outer-sphere solvents, counter-anions, and hydrogen atoms attached to carbon centers are omitted for clarity.

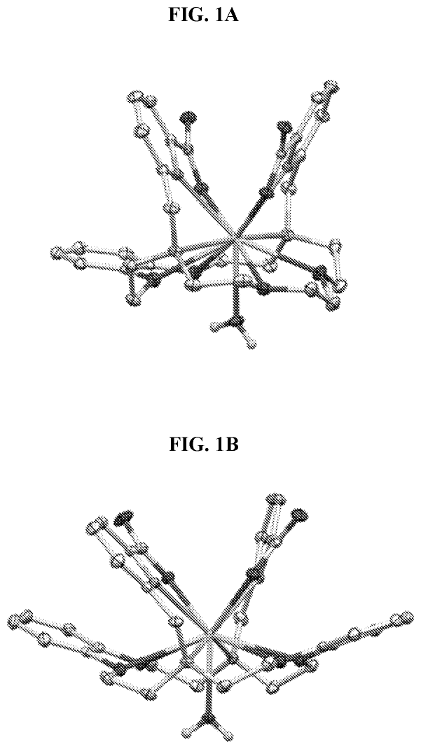

FIGS. 2A-2B shows the selected organ biodistribution of 2²⁵Ac-labeled GC33-BZM (solid), GC33-M (checkered), and OBI-BZM (striped) at 48 h (FIG. 2A) and 96 h (FIG. 2B) after administration (n=3-4) in mice.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure and claims will be understood to mean any one of the recited members individually or a combination of any two or more thereof—for example, “A, B, and/or C” would mean “A, B, C, A and B, A and C, B and C, or the combination of A, B, and C.”

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF5), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

As used herein, Cₘ-Cₙ, such as C₁-C₁₂, C₁-C₈, or C₁-C₆ when used before a group refers to that group containing m to n carbon atoms.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Alkyl groups may be substituted or unsubstituted. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Cycloalkyl groups may be substituted or unsubstituted. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Cycloalkylalkyl groups may be substituted or unsubstituted. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Alkenyl groups may be substituted or unsubstituted. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Alkynyl groups may be substituted or unsubstituted. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Aryl groups may be substituted or unsubstituted. The phrase “aryl groups” includes groups containing fused rings, such as fused aromatic-aliphatic ring systems (e.g., indanyl, tetrahydronaphthyl, and the like). Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Aralkyl groups may be substituted or unsubstituted. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Heterocyclyl groups may be substituted or unsubstituted. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Heteroaryl groups may be substituted or unsubstituted. Thus, the phrase “heteroaryl groups” includes fused ring compounds as well as includes heteroaryl groups that have other groups bonded to one of the ring members, such as alkyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene. Such groups may further be substituted or unsubstituted.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Alkoxy groups may be substituted or unsubstituted. Representative substituted alkoxy groups may be substituted one or more times with substituents such as those listed above.

The terms “alkanoyl” and “alkanoyloxy” as used herein can refer, respectively, to —C(O)-alkyl and —O—C(O)-alkyl groups, where in some embodiments the alkanoyl or alkanoyloxy groups each contain 2-5 carbon atoms. Similarly, the terms “aryloyl” and “aryloyloxy” respectively refer to —C(O)-aryl and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylic acid” as used herein refers to a compound with a —C(O)OH group. The term “carboxylate” as used herein refers to a —C(O)O⁻ group. A “protected carboxylate” refers to a —C(O)O-G where G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “ester” as used herein refers to —COOR⁷⁰ groups. R⁷⁰ is a substituted or unsubstituted alkyl, cycloalkyl, alkenyl, alkynyl, aryl, aralkyl, heterocyclylalkyl or heterocyclyl group as defined herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while sulfides include —SR⁸⁰ groups, sulfoxides include —S(O)R⁸¹ groups, sulfones include —SO₂R⁸² groups, and sulfonyls include —SO₂₀R⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “trifluoromethyldiazirido” refers to

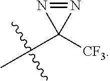 [SMILES: *C1(C(F)(F)F)N=N1]

The term “isocyano” refers to —NC.

The term “isothiocyano” refers to —NCS.

The term “pentafluorosulfanyl” refers to —SF₅.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid).

When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g. dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

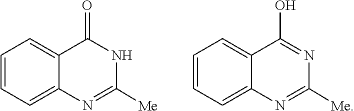 [SMILES: Cc1nc2ccccc2c(=O)[nH]1 ; Cc1nc(O)c2ccccc2n1]

As another example, guanidines may exhibit the following isomeric forms in protic organic solution, also referred to as tautomers of each other:

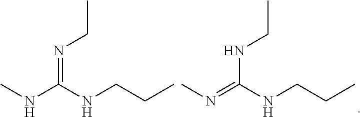

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

Throughout this disclosure, various publications, patents and published patent specifications are referenced by an identifying citation. Also within this disclosure are Arabic numerals referring to referenced citations, the full bibliographic details of which are provided immediately preceding the claims. The disclosures of these publications, patents and published patent specifications are hereby incorporated by reference into the present disclosure to more fully describe the present technology.

### The Present Technology

Although targeted radiotherapy has been practiced for some time using macrocyclic complexes of radionuclides, the macrocycles currently in use (e.g., DOTA) may form complexes of insufficient stability with radionuclides, particularly for radionuclides of larger size, such as actinium, radium, bismuth, and lead isotopes. Such instability results in dissociation of the radionuclide from the macrocycle, and this results in a lack of selectivity to targeted tissue, which also results in toxicity to non-targeted tissue. Accordingly, macrocyclic complexes that are substantially more stable than DOTA may be particularly beneficial in new therapies.

The present technology provides herein new macrocyclic complexes that are substantially more stable than those of the conventional art. For example, these new complexes may advantageously target cancer cells more effectively, with substantially less toxicity to non-targeted tissue than complexes of the art. Moreover, complexes described herein can advantageously be produced at room temperature, in contrast to DOTA-type complexes, which generally require elevated temperatures (e.g., at least 80° C.) for complexation with the radionuclide. The present technology may also employ alpha-emitting radionuclides instead of, e.g., beta radionuclides. Alpha-emitting radionuclides are of much higher energy, and thus substantially more potent, than beta-emitting radionuclides.

In one aspect, described herein is a compound of Formula (I):

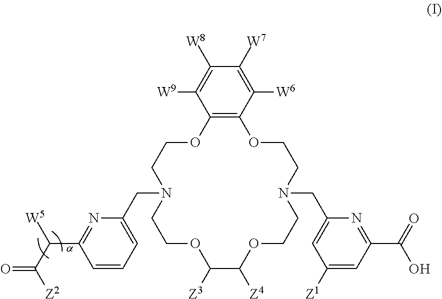

or a pharmaceutically acceptable salt and/or solvate thereof, wherein
Z¹ is H or —X¹—W¹;
Z² is OH or NH—W²;
Z³ is H or W³, and Z⁴ is H or W⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W¹, W², W⁶, W⁷, W⁸, and W⁹ are each independently H, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)_{y}—R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group;
W³, W⁴, W⁵, and W¹⁰ are each independently OH, NH₂, SH, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)yₓ-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group; and
R′ is independently at each occurrence H, halo, —N₃, C₁-C₆ alkyl, C₃-C₆ cycloalkyl, C₂-C₆ alkenyl, C₅-C₅ cycloalkenyl, C₂-C₆ alkynyl, C₅-C₁₀ cycloalkynyl, C₅-C₆ aryl, heterocyclyl, or heteroaryl.

Significantly, the uncomplexed form of Formula (I) may be complexed with a radionuclide, such as an alpha-emitting radionuclide, at room temperature (e.g., 18-30° C., or about or no more than 20° C., 25° C., or 30° C.) at high radiochemical yields, e.g., at least or greater than 90%, 95%, 97%, or 98%.

In a related aspect, a compound of Formula (II) is provided

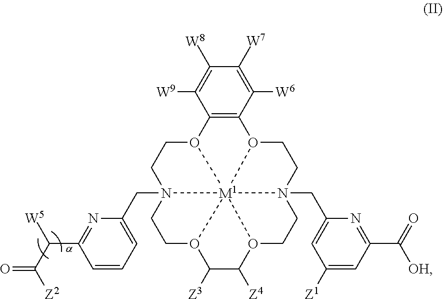

or a pharmaceutically acceptable salt and/or solvate thereof, wherein
M¹ is a radionuclide;
Z¹ is H or —X¹—W¹;
Z² is OH or NH—W²;
Z³ is H or W³, and Z⁴ is H or W⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W¹, W², W⁶, W⁷, W⁸, and W⁹ are each independently H, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)_{y}—R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group;
W³, W⁴, W⁵, and W¹⁰ are each independently OH, NH₂, SH, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)yₓ-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group; and
R′ is independently at each occurrence H, halo, —N₃, C₁-C₆ alkyl, C₃-C₆ cycloalkyl, C₂-C₆ alkenyl, C₅-C₅ cycloalkenyl, C₂-C₆ alkynyl, C₅-C₁₀ cycloalkynyl, C₅-C₆ aryl, heterocyclyl, or heteroaryl.

In any embodiment disclosed herein, it may be that M¹ is independently at each occurrence actinium-225 (²²⁵Ac³⁺), lanthanum-132 (¹³²La³⁺), lanthanum-135 (¹³⁵La³⁺), lutetium-177 (¹⁷⁷Lu³⁺), indium-111 (¹¹¹In³⁺), radium-223 (²³³Ra²⁺), bismuth-213 (²¹³Bi³⁺), lead-212 (2¹²Pb²⁺ and/or ²¹²Pb⁴⁺), terbium-149 (¹⁴⁹Tb³⁺), fermium-255 (²⁵⁵Fm³⁺), thorium-227 (²²⁷Th⁴⁺), thorium-226 (²²⁶Th⁴⁺), astatine-211 (²¹¹At), astatine-217 (²¹⁷At⁺), uranium-230, scandium-44 (⁴⁴Sc³⁺), scandium-47 (⁴⁷Sc³⁺), gallium-67 (⁶⁷Ga³⁺), or gallium-68 (⁶⁸Ga³⁺). In any embodiment disclosed herein, it may be that M¹ is actinium-225 (²²⁵Ac³⁺), radium-223 (²³³Ra²⁺), bismuth-213 (²¹³Bi³⁺), lead-212 (²¹²Pb²⁺ and/or ²¹²Pb⁴⁺), terbium-149 (¹⁴⁹Tb³⁺), fermium-255 (²⁵⁵Fm³⁺) thorium-227 (²²⁷Th⁴⁺), thorium-226 (²²⁶Th⁴⁺), astatine-211 (²¹¹At⁺), astatine-217 (²¹⁷At⁺), or uranium-230.

In a further related aspect, the present technology provides a compound (e.g., a “targeting compound”) useful in targeted radiotherapies, for example, targeted radiotherapy of cancer and/or mammalian tissue overexpressing, e.g., a glypican-3 (GPC3) receptor and/or prostate specific membrane antigen (“PSMA”), where the compound is of Formula (III)

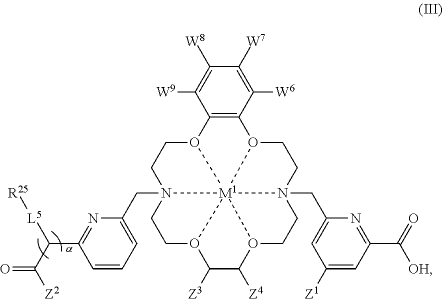

or a pharmaceutically acceptable salt and/or solvate thereof, wherein
M¹ is a radionuclide;
Z¹ is H or —X¹-L¹-R²¹;
Z² is OH or NH-L²-R²²;
Z³ is H or -L³-R²³, and Z⁴ is H or -L⁴-R²⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W⁶, W⁷, W⁸, W⁹, and W¹⁰ are each independently H or -L7-R²⁷;
L¹, L², L³, L⁴, L⁵, and L⁷ are each independently at each occurrence a bond or a linker group; and
R²¹, R²², R²³, R²⁴, R²⁵, and R²⁷ are each independently comprises an antibody, antibody fragment (e.g., an antigen-binding fragment), a binding moiety, a binding peptide, a binding polypeptide (such as a selective targeting oligopeptide containing up to 50 amino acids), a binding protein, an enzyme, a nucleobase-containing moiety (such as an oligonucleotide, DNA or RNA vector, or aptamer), or a lectin.

In any embodiment disclosed herein, it may be that M¹ is independently at each occurrence actinium-225 (²²⁵Ac³⁺), lanthanum-132 (¹³²La³⁺), lanthanum-135 (¹³⁵La³⁺), lutetium-177 (¹⁷⁷Lu³⁺), indium-111 (¹¹¹In³⁺), radium-223 (²³³Ra²⁺), bismuth-213 (²¹³Bi³⁺), lead-212 (²¹²Pb²⁺ and/or ²¹²Pb⁴⁺), terbium-149 (¹⁴⁹Tb³⁺), fermium-255 (²⁵⁵Fm³⁺), thorium-227 (²²⁷Th⁴⁺), thorium-226 (²²⁶Th⁴⁺), astatine-211 (²¹¹At⁺), astatine-217 (²¹⁷At⁺), uranium-230, scandium-44 (⁴⁴Sc³⁺), scandium-47 (⁴⁷Sc³⁺), gallium-67 (⁶⁷Ga³⁺), or gallium-68 (68Ga³⁺). In any embodiment disclosed herein, M¹ may be actinium-225 (²²⁵Ac³⁺), radium-223 (²³³Ra²⁺), bismuth-213 (²¹³Bi³⁺), lead-212 (²¹²Pb²⁺ and/or ²¹²Pb⁴⁺), terbium-149 (¹⁴⁹Tb³⁺), fermium-255 (²⁵⁵Fm³⁺) thorium-227 (²²⁷Th⁴⁺), thorium-226 (²²⁶Th⁴⁺), astatine-211 (²¹¹At⁺), astatine-217 (²¹⁷ At⁺), or uranium-230.

Representative R²¹, R²², R²¹, R²⁴, R²¹, and R²⁷ groups include those antibodies listed in Table A as well as antigen-binding fragments of such antibodies and any equivalent embodiments, as would be known to those of ordinary skill in the art.

TABLE A
Representative Antibodies

| | Disclosed In |
|---|---|
| Antibody | (U.S. Pat. No. or |
| (Trade Name(s)) | patent application Publ. No.)* |
| Belimumab | 7,138,501 |
| (Benlysta) | |
| Mogamulizumab | 6,989,145 |
| (Poteligeo) | |
| Blinatumomab | 7,112,324 |
| (Blincyto) | |
| Ibritumomab tiuxetan | 5,776,456 |
| (Zevalin) | |
| Obinutuzumab | 6,602,684 |
| (Gazyva) | |
| Ofatumumab¹ | 8,529,902 |
| (Arzerra) | |
| Rituximab | 5,736,137 |
| (Rituxan, MabThera) | |
| Inotuzumab ozogamicin | 8,153,768 |
| (Besponsa) | |
| Moxetumomab pasudotox | 8,809,502 |
| (Lumoxiti) | |
| Brentuximab vedotin | 7,829,531; 7,090,843 |
| (Adcetris) | |
| Daratumumab | 7,829,673 |
| (Darzalex) | |
| Ipilimumab | 6,984,720 |
| (Yervoy) | |
| Cetuximab | 6,217,866 |
| (Erbitux) | |
| Necitumumab | 7,598,350 |
| (Portrazza) | |
| Panitumumab | 6,235,883 |
| (Vectibix) | |
| Dinutuximab² | 7,432,357 |
| (Unituxin) | |
| Pertuzumab | 7,862,817 |
| (Perjeta, Omnitarg) | |
| Trastuzumab³ | 5,821,337 |
| (Herceptin) | |
| Trastuzumab emtansine | 7,097,840 |
| (Kadcyla) | |
| Siltuximab | 7,612,182 |
| (Sylvant) | |
| Cemiplimab⁴ | 9,987,500 |
| (Libtayo) | |
| Nivolumab | 8,008,449 |
| (Opdivo) | |
| Pembrolizumab | 8,354,509 |
| (Keytruda) | |
| Olaratumab | 8,128,929 |
| (Lartruvo) | |
| Atezolizumab | 8,217,149 |
| (Tecentriq) | |
| Avelumab⁵ | 9,624,298 |
| (Bavencio) | |
| Durvalumab | 8,779,108 |
| (Imfinzi) | |
| Capromab pendetide | 5,162,504 |
| (Prostascint) | |
| Elotuzumab | 7,709,610 |
| (Empliciti) | |
| Denosumab | 6,740,522 |
| (Prolia, Xgeva) | |
| Ziv-aflibercept | 7,070,959 |
| (Zaltrap) | |
| Bevacizumab | 6,054,297 |
| (Avastin) | |
| Ramucirumab | 7,498,414 |
| (Cyramza) | |
| Tositumomab | 6,565,827; 6,287,537;, 6,090,365; |
| (Bexxar) | 6,015,542; 5,843,398; 5,595,721 |
| Gemtuzumab ozogamicin | 5,773,001 |
| (Mylotarg) | |
| Alemtuzumab | 6,569,430; 5,846,534 |
| (Campath-1H) | |
| Cixutumumab | 7,968,093; 7,638,605 |
| Girentuximab | 8,466,263 |
| (Rencarex) | |
| Nimotuzumab | 6,506,883 |
| (Theracim, Theraloc) | |
| Catumaxomab | 9,017,676; 8,663,638; 2013/0309234A1 |
| (Removab) | |
| Etaracizumab | 2004/0001835A1 |
| (Abegrin, Vitaxin) | |

¹Also designated 2F2.
²Also designated Ch14.18.
³Also designated HuMaB4D5-8.
⁴Also designated H4H7798N.
⁵Also designated A09-246-2.
*Note:
the disclosures of the each of the patents and patent publications listed in Table A are incorporated herein by reference.

In any embodiment disclosed herein, it may be that the binding peptide comprises a prostate specific membrane antigen (“PSMA”) binding peptide, a somatostatin receptor agonist, a bombesin receptor agonist, a seprase binding compound, or a binding fragment thereof. Exemplary PSMA binding peptides include, but are not limited to, those according to the following structure

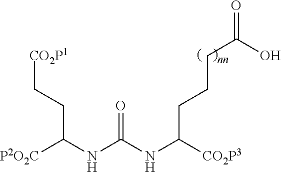

where nn is 0, 1, or 2, and P¹, P², and P³ are each independently H, methyl, benzyl, 4-methoxybenzyl, or tert-butyl. In any embodiment herein, it may be that each of P¹, P², and P³ are H.

Somatostatin, illustrated in Scheme A, is a peptide hormone that regulates the endocrine system and affects neurotransmission and cell proliferation via interaction with G protein-coupled somatostatin receptors and inhibition of the release of numerous secondary hormones. Somatostatin has two active forms produced by alternative cleavage of a single preproprotein. There are five known somatostatin receptors, all being G protein-coupled seven transmembrane receptors: SST1 (SSTR1); SST2 (SSTR2); SST3 (SSTR3); SST4 (SSTR4); and SST5 (SSTR5). Exemplary somatostatin receptor agonists include somatostatin itself, lanreotide, octreotate, octreotide, pasireotide, and vapreotide.

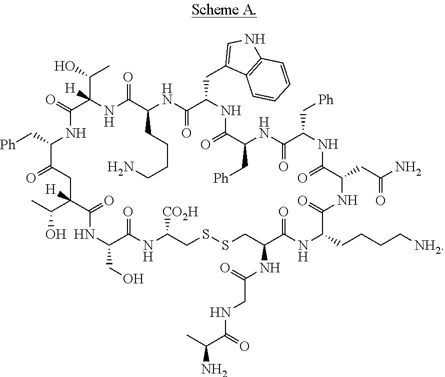 [SMILES: C[C@H](N)C(=O)NCC(=O)N[C@H]1CSSC[C@@H](C(=O)O)NC(=O)[C@H](CO)NC(=O)[C@H]([C@@H](C)O)CC(=O)[C@H](Cc2ccccc2)NC(=O)[C@H]([C@@H](C)O)NC(=O)[C@H](CCCCN)NC(=O)[C@H](Cc2c[nH]c3ccccc23)NC(=O)[C@H](Cc2ccccc2)NC(=O)[C@H](Cc2ccccc2)NC(=O)[C@H](CC(N)=O)NC(=O)[C@H](CCCCN)NC1=O]

Many neuroendocrine tumors express SSTR2 and the other somatostatin receptors. Long acting somatostatin agonists (e.g., Octreotide, Lanreotide) are used to stimulate the SSTR2 receptors, and thus to inhibit further tumor proliferation. See, Zatelli M C, et al., (Apr 2007). “Control of pituitary adenoma cell proliferation by somatostatin analogs, dopamine agonists and novel chimeric compounds”. European Journal of Endocrinology/European Federation of Endocrine Societies. 156 Suppl 1: S29-35. Octreotide is an octapeptide that mimics natural somatostatin but has a significantly longer half-life in vivo. Octreotide is used for the treatment of growth hormone producing tumors (acromegaly and gigantism), when surgery is contraindicated, pituitary tumors that secrete thyroid stimulating hormone (thyrotropinoma), diarrhea and flushing episodes associated with carcinoid syndrome, and diarrhea in people with vasoactive intestinal peptide-secreting tumors (VIPomas). Lanreotide is used in the management of acromegaly and symptoms caused by neuroendocrine tumors, most notably carcinoid syndrome. Pasireotide is a somatostatin analog with an increased affinity to SSTR5 compared to other somatostatin agonists and is approved for treatment of Cushing's disease and acromegaly. Vapreotide is used in the treatment of esophageal variceal bleeding in patients with cirrhotic liver disease and AIDS-related diarrhea.

Bombesin is a peptide originally isolated from the skin of the European fire-bellied toad (Bombina bombina). In addition to stimulating gastrin release from G cells, bombesin activates at least three different G-protein-coupled receptors: BBR1, BBR2, and BBR3, where such activity includes agonism of such receptors in the brain. Bombesin is also a tumor marker for small cell carcinoma of lung, gastric cancer, pancreatic cancer, and neuroblastoma. Bombesin receptor agonists include, but are not limited to, BBR-1 agonists, BBR-2 agonists, and BBR-3 agonists.

Seprase (or Fibroblast Activation Protein (FAP)) is an integral membrane serine peptidase. In addition to gelatinase activity, seprase has a dual function in tumour progression. Seprase promotes cell invasiveness towards the ECM and also supports tumour growth and proliferation. Seprase binding compounds include seprase inhibitors.

Glypican-3 (GPC3) is a cell-surface glycoprotein consisting of heparan sulfate glycosaminoglycan chains and an inner protein core. Glypican 3 immunostaining has utility for differentiating hepatocellular carcinoma (HCC) (see, e.g., Filmus J, Capurro M (2004). “Glypican-3 and alphafetoprotein as diagnostic tests for hepatocellular carcinoma”. Molecular Diagnosis. 8 (4): 207-212) and dysplastic changes in cirrhotic livers; HCC stains with glypican 3, while liver with dysplastic changes and/or cirrhotic changes does not. GPC3 protein expression has been found in HCC, not in normal liver and cholangiocarcinoma. GPC3 is also expressed to a lesser degree in melanoma, ovarian clear-cell carcinomas, yolk sac tumors, neuroblastoma, hepatoblastoma, Wilms' tumor cells, and other tumors. GPC3 is a promising therapeutic target for treating liver cancer (see, e.g., Ishiguro T, Sugimoto M, Kinoshita Y, Miyazaki Y, Nakano K, Tsunoda H, et al. “Anti-glypican 3 antibody as a potential antitumor agent for human liver cancer”. Cancer Research. 68 (23): 9832-9838).

In a further related aspect, a modified antibody, modified antibody fragment, or modified binding peptide comprising a linkage arising from conjugation of a compound of Formula (I) or pharmaceutically acceptable salt and/or solvate thereof, with an antibody, antibody fragment, or binding peptide. In a related aspect, a modified antibody, modified antibody fragment, or modified binding peptide is provided that includes a linkage arising from conjugation of a compound of Formula (II) or a pharmaceutically acceptable salt and/or solvate thereof, with an antibody, antibody fragment, or binding peptide. In any embodiment disclosed herein, it may be that the antibody includes belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, or Etaracizumab. In any embodiment disclosed herein, it may be that the antibody fragment includes an antigen-binding fragment of belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, or Etaracizumab. In embodiments, it may be that the binding peptide includes a prostate specific membrane antigen (“PSMA”) binding peptide, a somatostatin receptor agonist, a bombesin receptor agonist, a seprase binding compound, or a binding fragment thereof. In embodiments, it may be that the binding peptide includes Codrituzumab (GC33) or a binding fragment thereof.

As an example of a modified antibody, modified antibody fragment, or modified binding peptide of the present technology, it may be that the linkage is a thiocyante linkage; wherein the thiocyanate linkage arises from conjugation of the compound with the antibody, antibody fragment, or binding peptide; and wherein the compound is

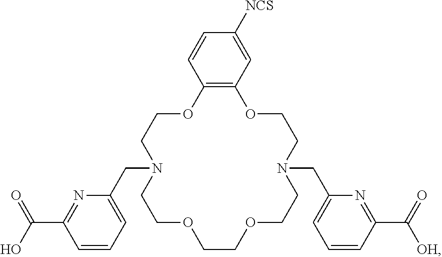

or pharmaceutically acceptable salt and/or solvate thereof.

As another example of a modified antibody, modified antibody fragment, or modified binding peptide of the present technology, it may be that the linkage is a thiocyante linkage; wherein the thiocyanate linkage arises from conjugation of the compound with the antibody, antibody fragment, or binding peptide; and wherein the compound is

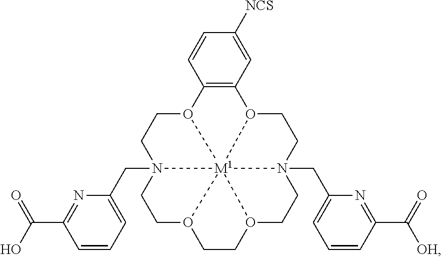

or a pharmaceutically acceptable salt and/or solvate thereof.

In any embodiment herein, it may be that the structures include compounds of Formula (I-A); a modified antibody, modified antibody fragment, or modified binding peptide comprising a linkage arising from conjugation of a compound of Formula (I-A) or pharmaceutically acceptable salt and/or solvate thereof, with an antibody, antibody fragment, or binding peptide; compounds of Formula (II-A); a modified antibody, modified antibody fragment, or modified binding peptide comprising a linkage arising from conjugation of a compound of Formula (II-A) or pharmaceutically acceptable salt and/or solvate thereof, with an antibody, antibody fragment, or binding peptide; and targeting compounds of Formula (III-A)

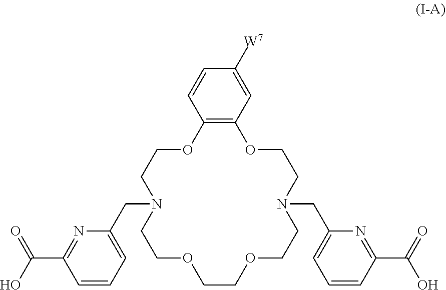

or a pharmaceutically acceptable salt and/or solvate thereof,

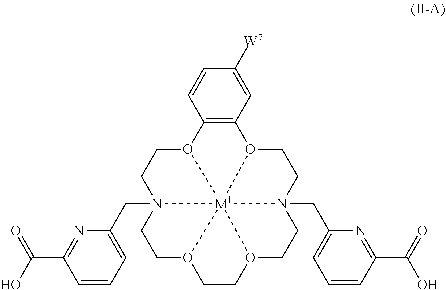

or a pharmaceutically acceptable salt and/or solvate thereof,

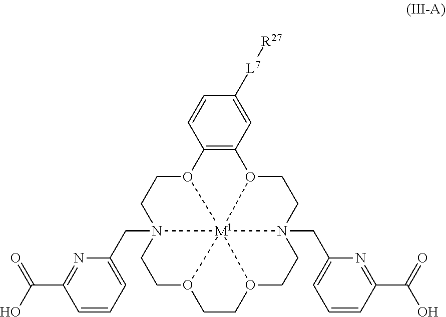

or a pharmaceutically acceptable salt and/or solvate thereof, wherein M¹ is independently at each occurrence a radionuclide.

Targeting compounds of Formula (III-A) may be prepared by a process that includes reacting a compound of Formula (I-A) or (II-A) with R²⁷—W^{7a}, where Table B provides representative examples (where n is independently at each occurrence 1, 2, 3, 4, 5, 6, 7, 8, 9, or 10). As such, R²⁷ may be conjugated to macrocycle R^{4′} by reaction of complementary chemical functional groups W^{7a} and W⁷ (e.g., represented by —X²—W^{7′} in Table B) to form linker L⁷ (e.g., represented by —X²-L^{7′} in Table B). For example, R²⁷—W^{7a} may include a modified target amino acid residue within a protein (e.g., one of the representative antibodies disclosed in Table A or an antigen-binding fragment thereof, a PSMA binding peptide, a somatostatin receptor agonist, a bombesin receptor agonist, a seprase binding compound, or a binding fragment of any one thereof, or an antibody codrituzumab (GC33), or a binding fragment of any one thereof). W^{7a} may include a reactive chemical functional moiety, non-limiting examples of which are disclosed in the Table B, where W⁷ (e.g., represented by —X²—W^{7′} in Table B) may be selected to selectively react with W^{7a} in order to provide L⁷ (e.g., represented by —X²-L^{7′} in Table B) of Formula (III-A). In some embodiments, X² is O, NH, S, or a covalent bond. In some embodiments, W^{7′} is H, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)_{y}—R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group;

TABLE B

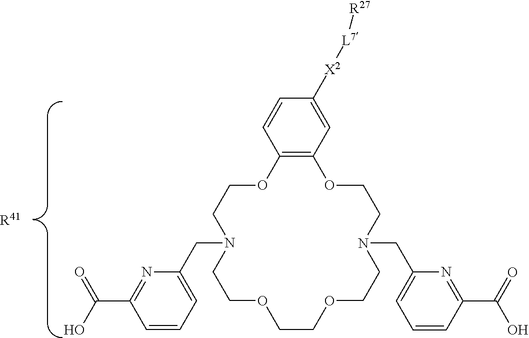

| | R⁴¹—W⁷ | | Final Conjugation Product |
|---|---|---|---|
| W^{7a}—R²⁷ | R⁴¹—X²—W^{7′} | X² | (R⁴¹—X²—L⁷′—R²⁷) |
| H₂N—R²⁷ | R⁴¹—NCS | N/A | 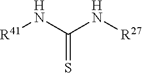 |
| N₃—R²⁷ | 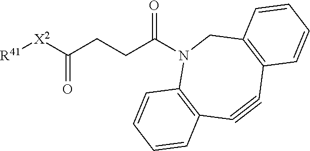 | NH | 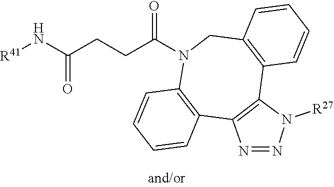 |
| | | | 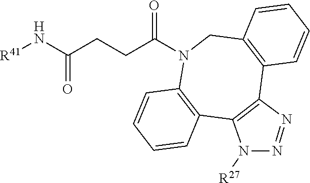 |
| | | O | 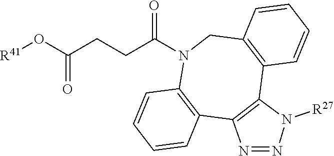 |
| | | | and/or |
| | | | 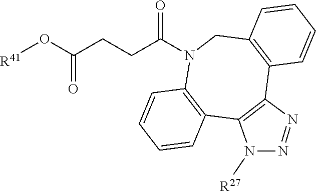 |
| | 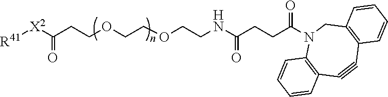 | | 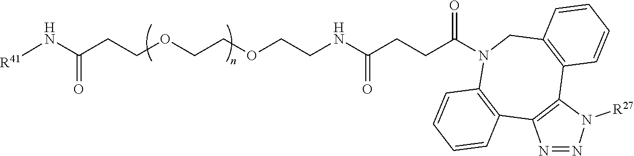 |
| | | | and/or |
| | | | 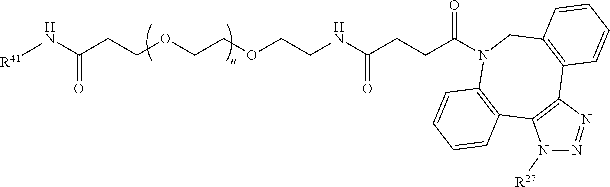 |
| | | O | 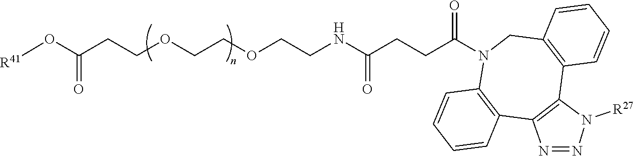 |
| | | | and/or |
| | | | 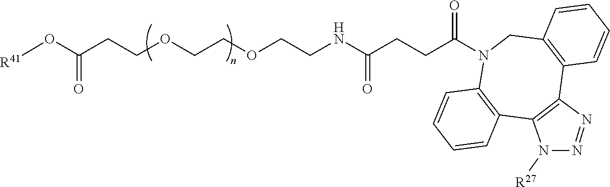 |
| 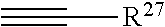 | 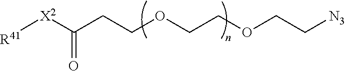 | NH | 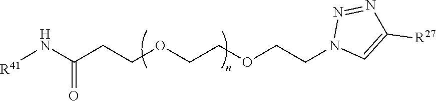 |
| | | | and/or |
| | | | 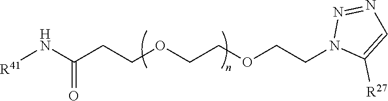 |
| | | O | 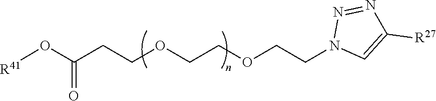 |
| | | | and/or |
| | | | 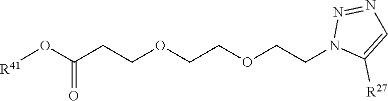 |
| | 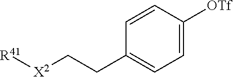 | NH | 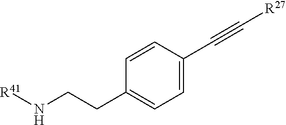 |
| | | O | 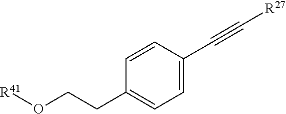 |
| | 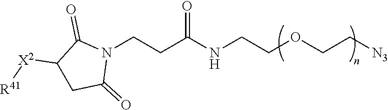 | S | 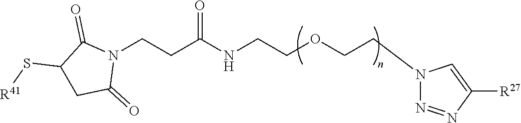 |
| 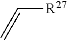 | 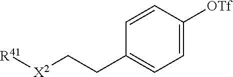 | NH | 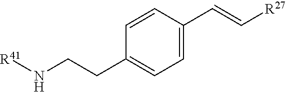 |
| | | O | 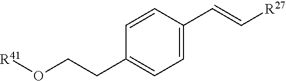 |
| 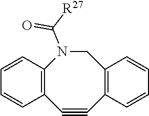 | 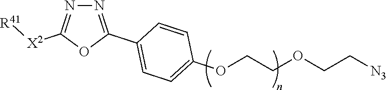 | S | 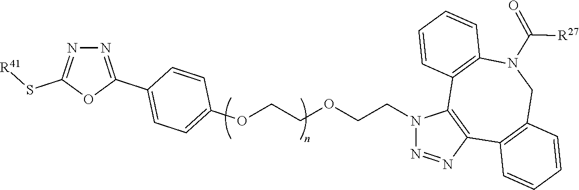 |
| | | | and/or |
| | | | 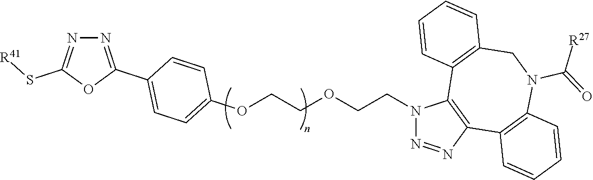 |
| 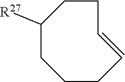 | 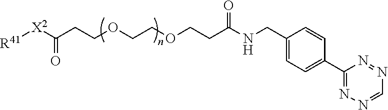 | NH | 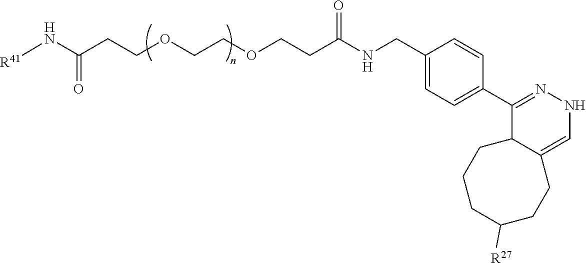 |
| | | | and/or |
| | | | 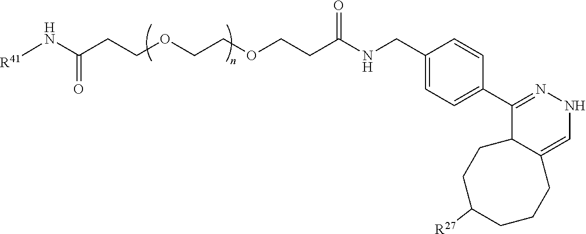 |
| | | O | 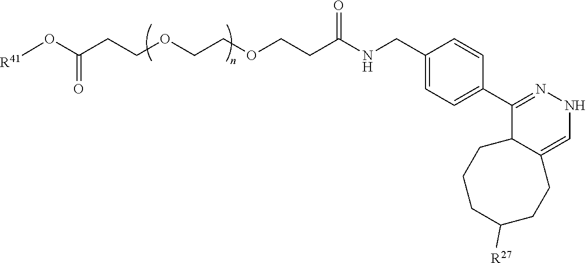 |
| | | | and/or |
| | | | 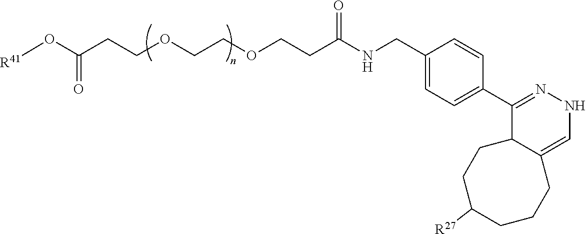 |
| 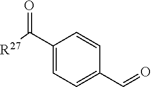 | 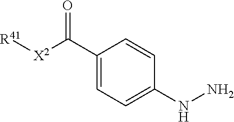 | NH | 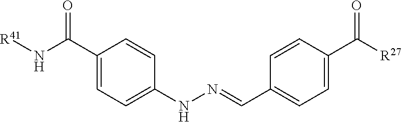 |
| | | O | 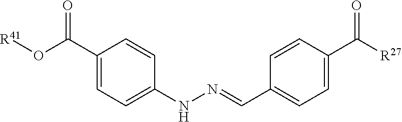 |
| H₂N—R²⁷ | 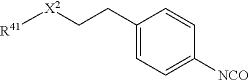 | NH | 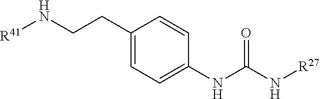 |
| | | O | 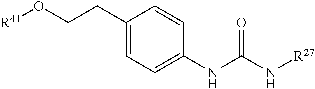 |
| | 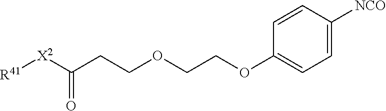 | NH | 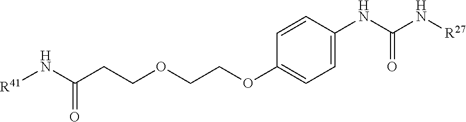 |
| | | O | 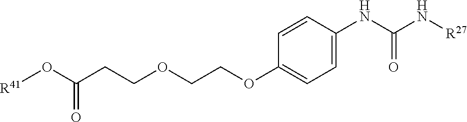 |
| | 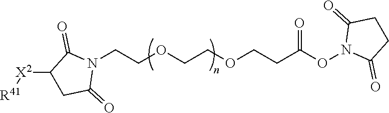 | S | 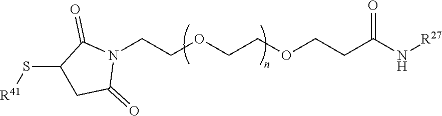 |
| | 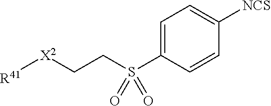 | | 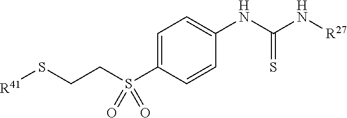 |
| | 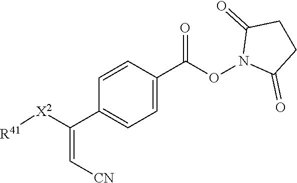 | | 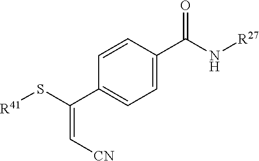 |
| 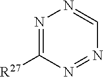 | 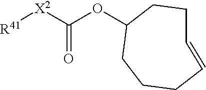 | NH | 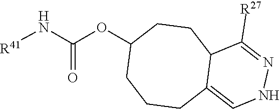 |
| | | | and/or |
| | | | 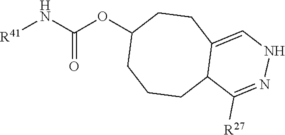 |
| | | O | 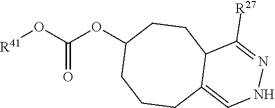 |
| | | | and/or |
| | | | 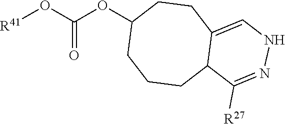 |
| | 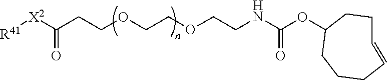 | NH | 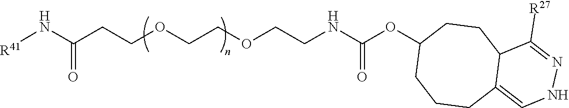 |
| | | | and/or |
| | | | 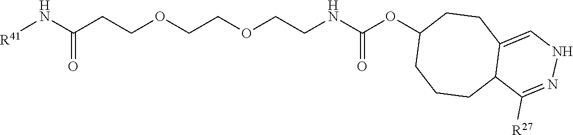 |
| | | O | 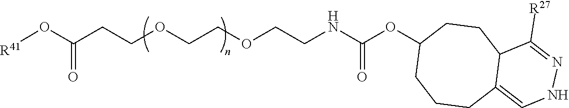 |
| | | | and/or |
| | | | 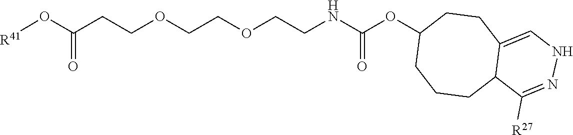 |
| | 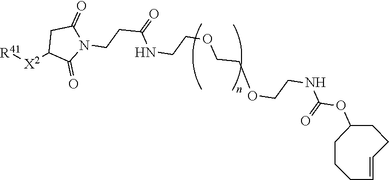 | S | 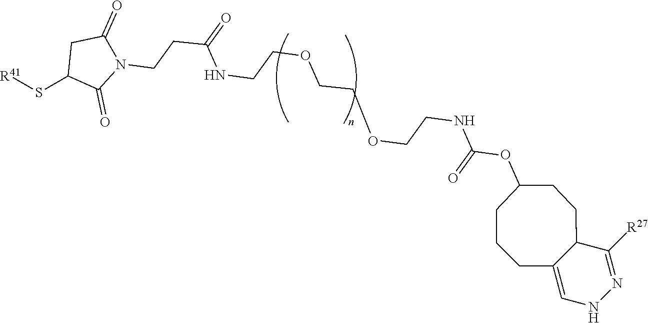 |
| | | | and/or |
| | | | 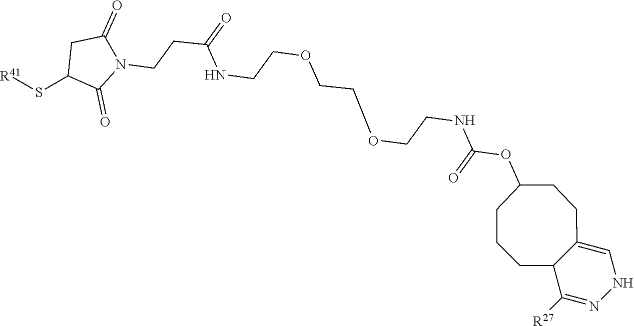 |
| 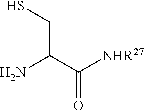 | 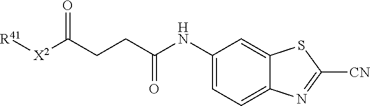 | NH | 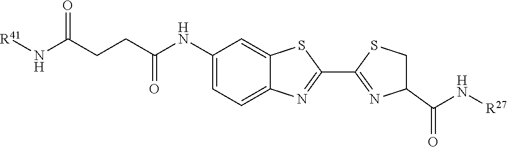 |
| | | O | 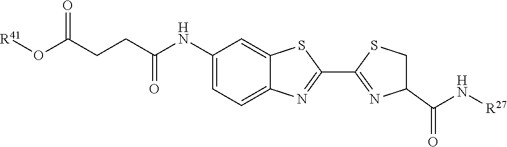 |
| | 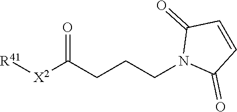 | NH | 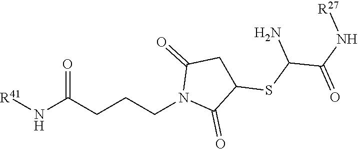 |
| | | O | 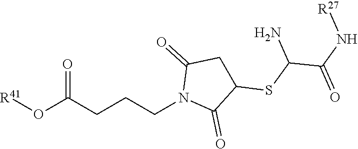 |
| 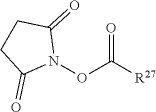 | 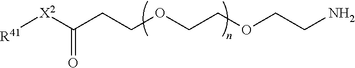 | NH | 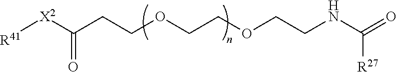 |
| | | O | 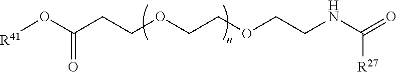 |
| | 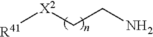 | NH | 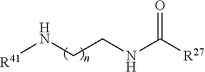 |
| | | O | 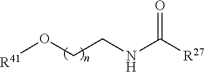 |

In any embodiment herein, it may be that the structures include compounds of Formula (I-B); a modified antibody, modified antibody fragment, or modified binding peptide comprising a linkage arising from conjugation of a compound of Formula (I-B) or pharmaceutically acceptable salt and/or solvate thereof, with an antibody, antibody fragment, or binding peptide; compounds of Formula (II-B); a modified antibody, modified antibody fragment, or modified binding peptide comprising a linkage arising from conjugation of a compound of Formula (II-B) or pharmaceutically acceptable salt and/or solvate thereof, with an antibody, antibody fragment, or binding peptide; and targeting compounds of Formula (III-B)

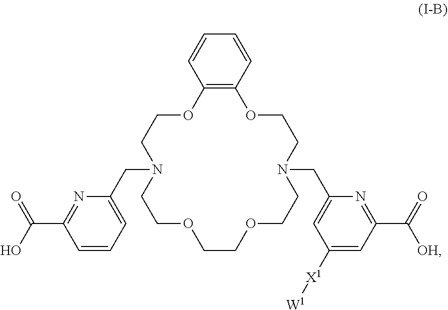

or a pharmaceutically acceptable salt and/or solvate thereof,

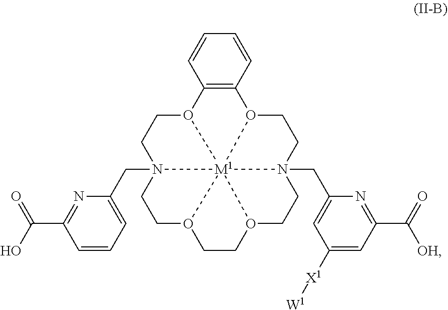

or a pharmaceutically acceptable salt and/or solvate thereof,

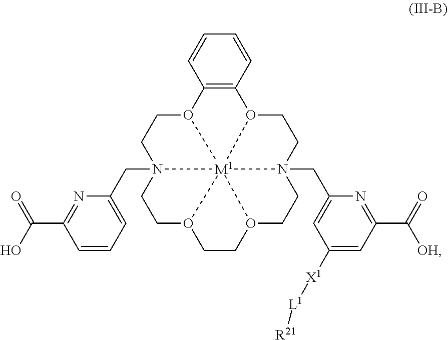

or a pharmaceutically acceptable salt and/or solvate thereof, wherein M¹ is independently at each occurrence a radionuclide.

Targeting compounds of Formula (III-B) may be prepared by a process that includes reacting a compound of Formula (I-B) or (II-B) with R²¹—W^{1a}, where Table C provides representative examples (where n is independently at each occurrence 1, 2, 3, 4, 5, 6, 7, 8, 9, or 10). As such, R²¹ may be conjugated to macrocycle R⁴² by reaction of complementary chemical functional groups W¹ and W^{1a} to form linker L¹. For example, R²¹—W^{1a} may include a modified target amino acid residue within a protein (e.g., one of the representative antibodies disclosed in Table A or an antigen-binding fragment thereof, a PSMA binding peptide, a somatostatin receptor agonist, a bombesin receptor agonist, a seprase binding compound, or a binding fragment of any one thereof, or an antibody codrituzumab (GC33), or a binding fragment of any one thereof). W¹ may include a reactive chemical functional moiety, non-limiting examples of which are disclosed in the Table C, where W¹ may be selected to selectively react with W^{1a} in order to provide L¹ of Formula (III-B).

TABLE C

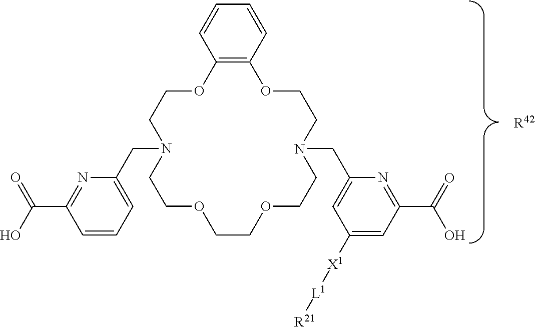

| | | | Final Conjugation Product |
|---|---|---|---|
| W^{1a}—R²¹ | R⁴²—X¹—W¹ | X¹ | (R⁴2—X¹—L¹—R²¹) |
| N³—R²¹ | 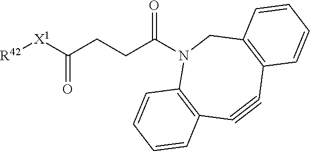 | N H | 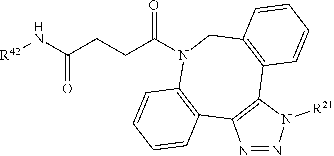 |
| | | | and/or |
| | | | 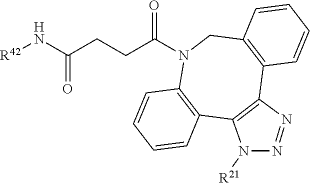 |
| | | O | 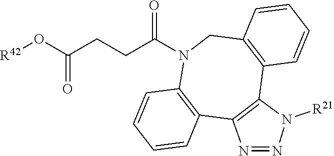 |
| | | | and/or |
| | | | 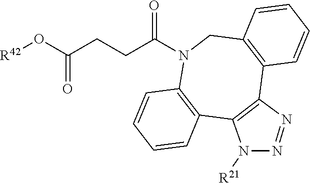 |
| | 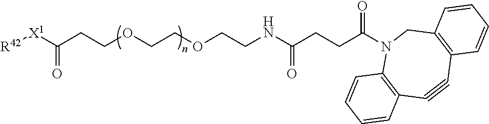 | N H | 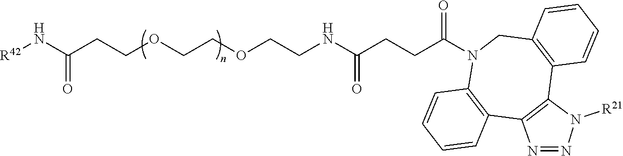 |
| | | | and/or |
| | | | 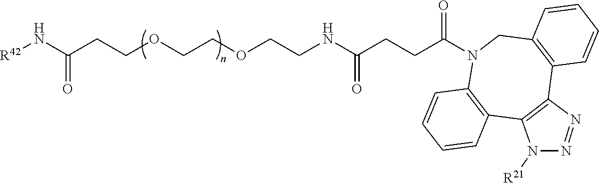 |
| | | O | 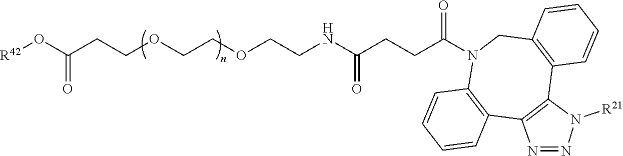 |
| | | | and/or |
| | | | 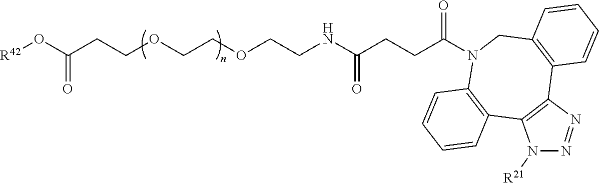 |
| 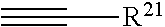 | 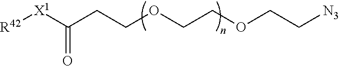 | N H | 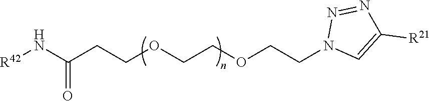 |
| | | | and/or |
| | | | 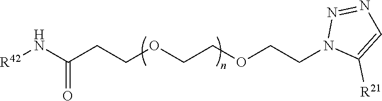 |
| | | O | 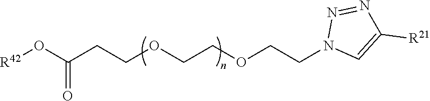 |
| | | | and/or |
| | | | 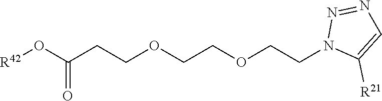 |
| | 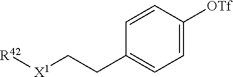 | N H | 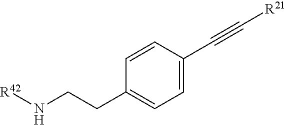 |
| | | O | 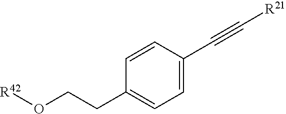 |
| | 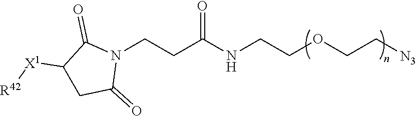 | S | 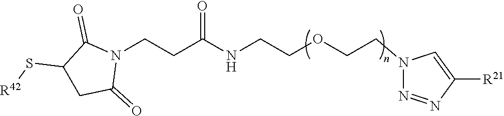 |
| 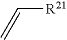 | | N H | 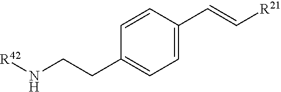 |
| | 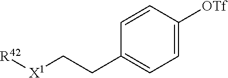 | O | 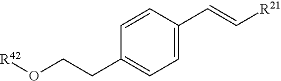 |
| 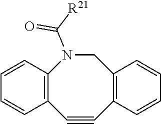 | 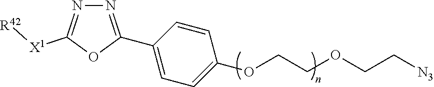 | S | 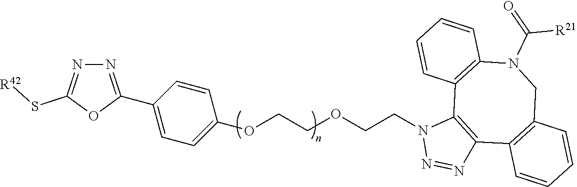 |
| | | | and/or |
| | | | 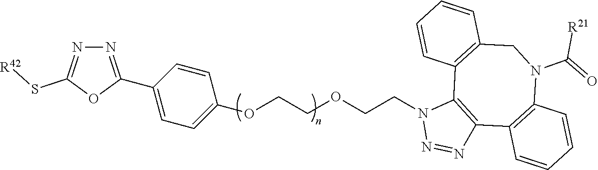 |
| 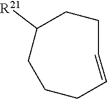 | 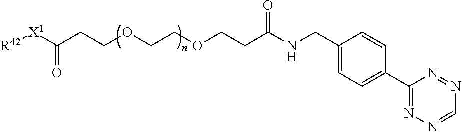 | N H | 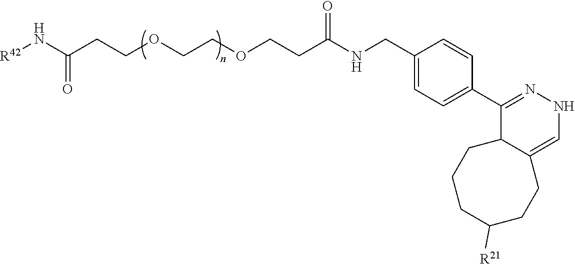 |
| | | | and/or |
| | | | 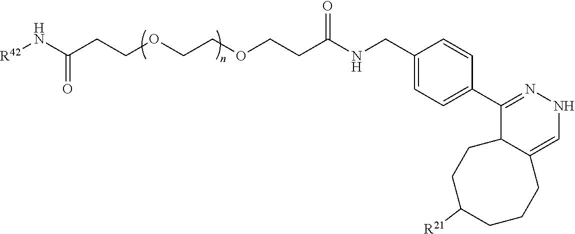 |
| | | O | 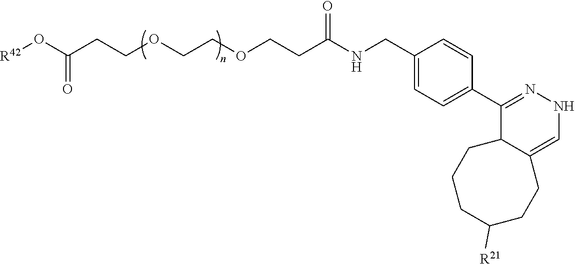 |
| | | | and/or |
| | | | 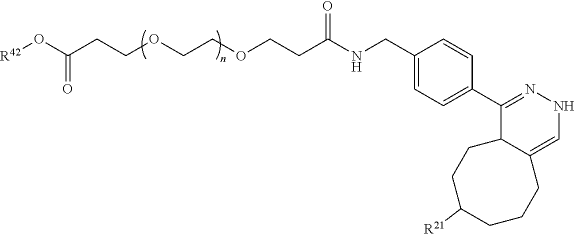 |
| 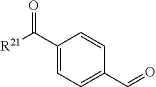 | 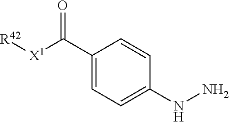 | N H | 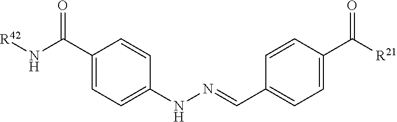 |
| | | O | 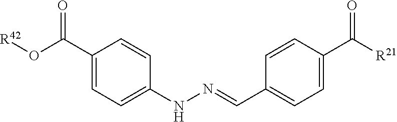 |
| H₂N—R²¹ | 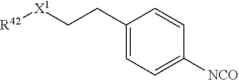 | N H | 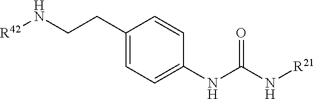 |
| | | O | 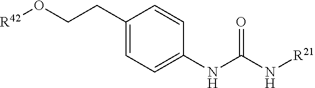 |
| | 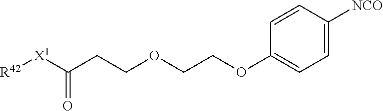 | N H | 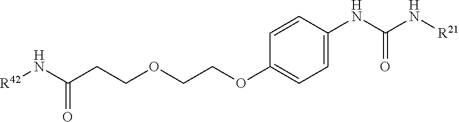 |
| | | O | 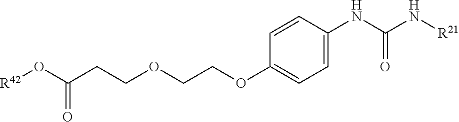 |
| | 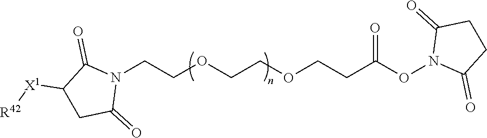 | S | 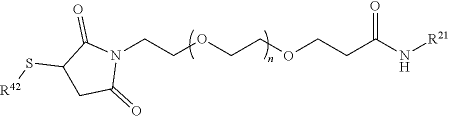 |
| | 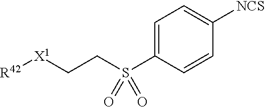 | | 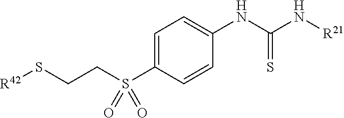 |
| | 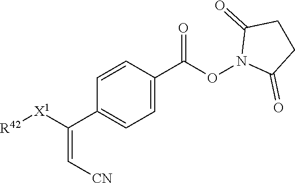 | | 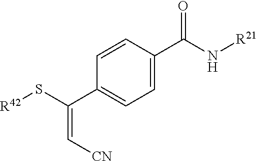 |
| 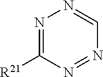 | 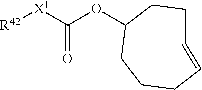 | N H | 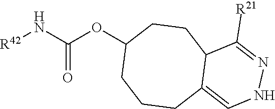 |
| | | | and/or |
| | | | 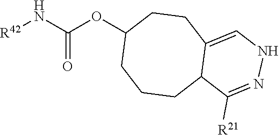 |
| | | O | 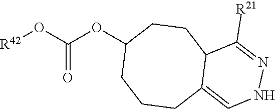 |
| | | | and/or |
| | | | 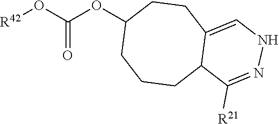 |
| | 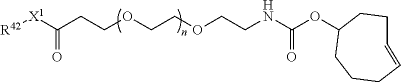 | N H | 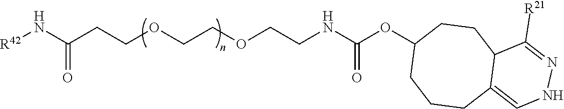 |
| | | | and/or |
| | | | 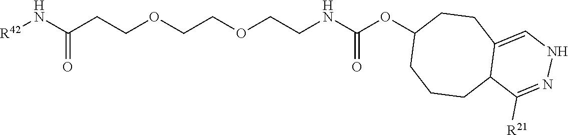 |
| | | O | 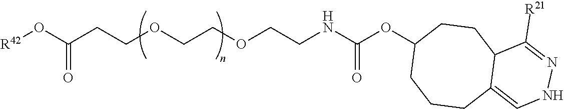 |
| | | | and/or |
| | | | 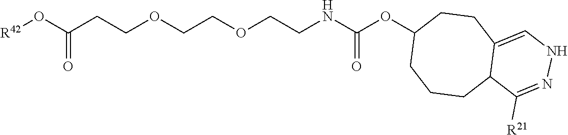 |
| | 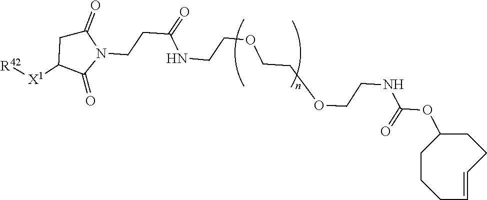 | S | 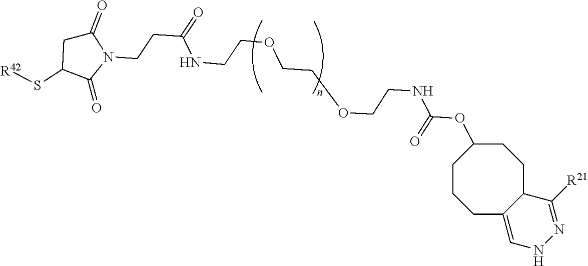 |
| | | | and/or |
| | | | 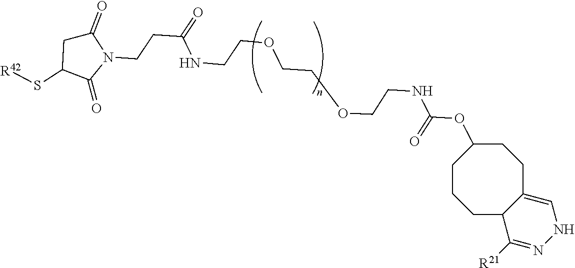 |
| 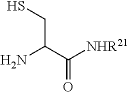 | 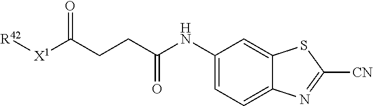 | N H | 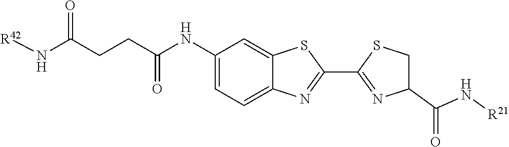 |
| | | O | 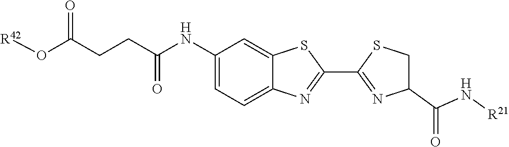 |
| | 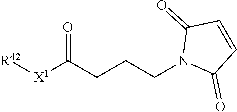 | N H | 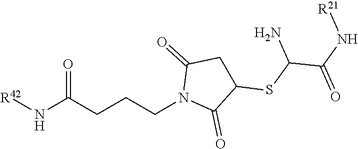 |
| | | O | 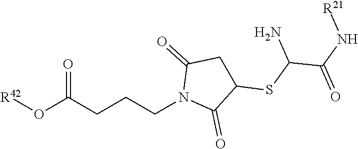 |
| 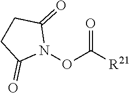 | 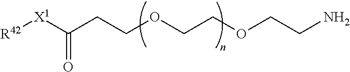 | N H | 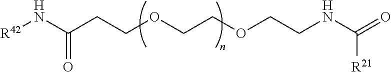 |
| | | O | 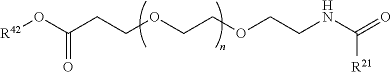 |
| | 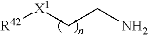 | N H | 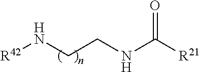 |
| | | O | 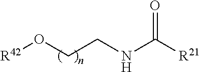 |

In any embodiment herein, it may be that the structures include compounds of Formula (I-C); a modified antibody, modified antibody fragment, or modified binding peptide comprising a linkage arising from conjugation of a compound of Formula (I-C) or pharmaceutically acceptable salt and/or solvate thereof, with an antibody, antibody fragment, or binding peptide; compounds of Formula (II-C); a modified antibody, modified antibody fragment, or modified binding peptide comprising a linkage arising from conjugation of a compound of Formula (II-C) or pharmaceutically acceptable salt and/or solvate thereof, with an antibody, antibody fragment, or binding peptide; and targeting compounds of Formula (III-C)

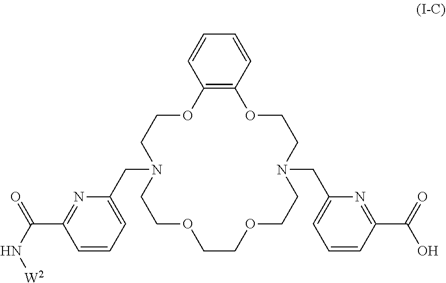

or a pharmaceutically acceptable salt and/or solvate thereof,

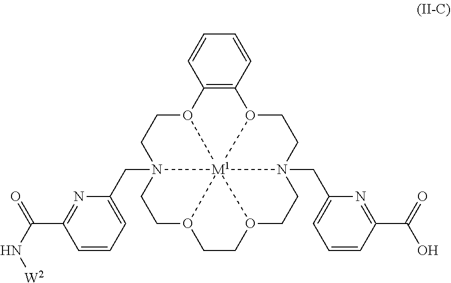

or a pharmaceutically acceptable salt and/or solvate thereof,

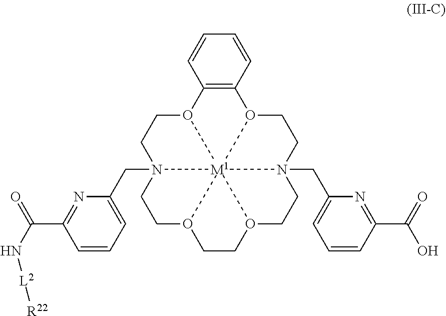

or a pharmaceutically acceptable salt and/or solvate thereof, wherein M is independently at each occurrence a radionuclide.

Targeting compounds of Formula (III-C) may be prepared by a process that includes reacting a compound of Formula (I-C) or (II-C) with R²²—W^{2a}, where Table D provides representative examples (where n is independently at each occurrence 1, 2, 3, 4, 5, 6, 7, 8, 9, or 10). As such, R²² may be conjugated to macrocycle R⁴³ by reaction of complementary chemical functional groups W² and W^{2a} to form linker L². For example, R²²—W^{2a} may include a modified target amino acid residue within a protein (e.g., one of the representative antibodies disclosed in Table A or an antigen-binding fragment thereof, a PSMA binding peptide, a somatostatin receptor agonist, a bombesin receptor agonist, a seprase binding compound, or a binding fragment of any one thereof, or an antibody codrituzumab (GC33), or a binding fragment of any one thereof). W² may include a reactive chemical functional moiety, non-limiting examples of which are disclosed in the Table D, where W² may be selected to selectively react with W^{2a} in order to provide L² of Formula (III-C).

TABLE D

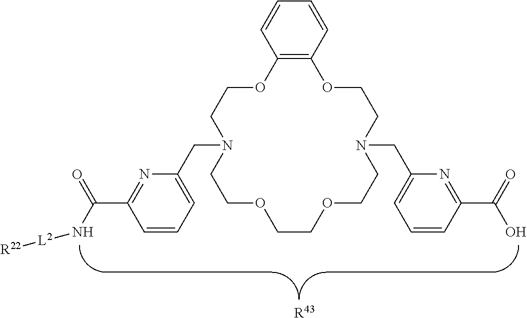

| | | Final Conjugation Product |
|---|---|---|
| R²²—W^{2a} | W²—R⁴³ | (R²²—L²—R⁴³) |
| 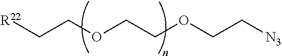 | 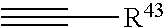 | 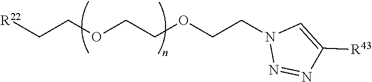 |
| | | and/or |
| | | 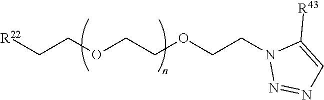 |
| | 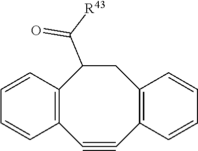 | 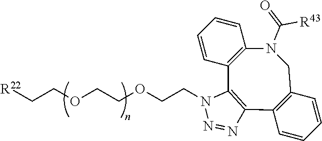 |
| | | and/or |
| | | 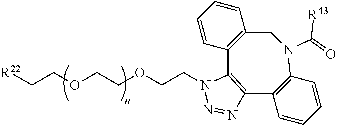 |
| 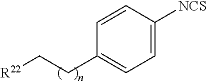 | H₂N—R⁴³ | 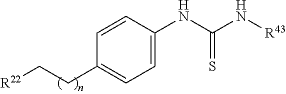 |
| 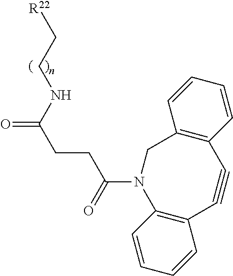 | N₃—R⁴³ | 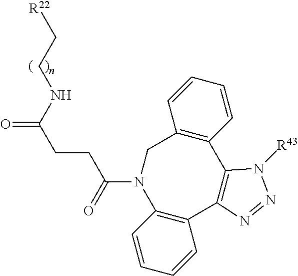 |
| | | and/or |
| | | 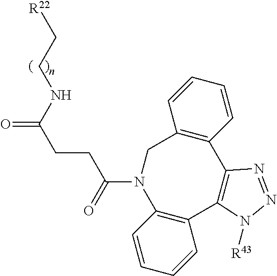 |
| 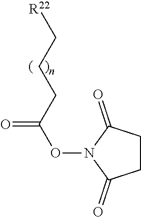 | H₂N—R⁴³ | 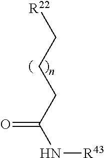 |
| 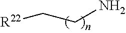 | 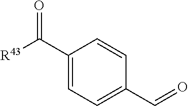 | 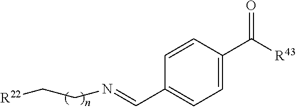 |
| | 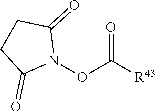 | 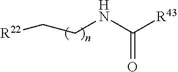 |
| 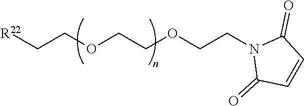 | 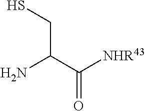 | 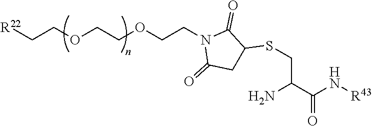 |

In any embodiment herein, it may be that the structures include compounds of Formula (I-D); a modified antibody, modified antibody fragment, or modified binding peptide comprising a linkage arising from conjugation of a compound of Formula (I-D) or pharmaceutically acceptable salt and/or solvate thereof, with an antibody, antibody fragment, or binding peptide; compounds of Formula (II-D); a modified antibody, modified antibody fragment, or modified binding peptide comprising a linkage arising from conjugation of a compound of Formula (II-D) or pharmaceutically acceptable salt and/or solvate thereof, with an antibody, antibody fragment, or binding peptide; and targeting compounds of Formula (III-D)

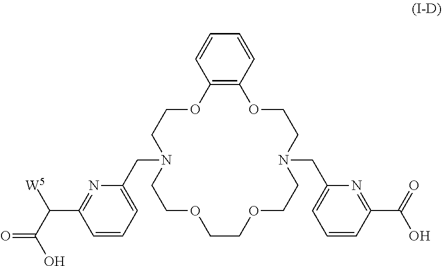

or a pharmaceutically acceptable salt and/or solvate thereof,

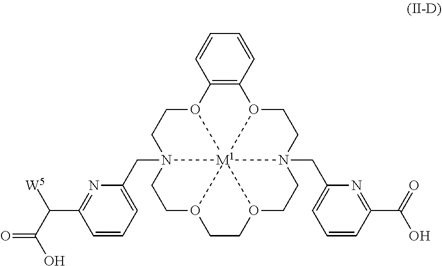

or a pharmaceutically acceptable salt and/or solvate thereof,

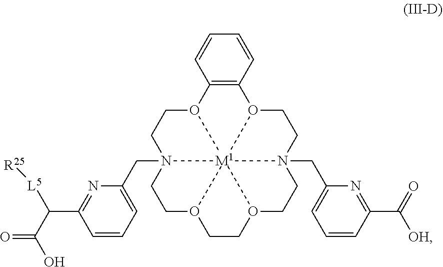

or a pharmaceutically acceptable salt and/or solvate thereof, wherein M¹ is independently at each occurrence a radionuclide.

Targeting compounds of Formula (III-D) may be prepared by a process that includes reacting a compound of Formula (I-D) or (II-D) with R²⁵—W^{5a}, where Table E provides representative examples (where n is independently at each occurrence 1, 2, 3, 4, 5, 6, 7, 8, 9, or 10). As such, R²⁵ may be conjugated to macrocycle R⁴⁴ by reaction of complementary chemical functional groups W⁵ and W^{5a} to form linker L⁵. For example, R²⁵—W^{5a} may include a modified target amino acid residue within a protein (e.g., one of the representative antibodies disclosed in Table A or an antigen-binding fragment thereof, a PSMA binding peptide, a somatostatin receptor agonist, a bombesin receptor agonist, a seprase binding compound, or a binding fragment of any one thereof, or an antibody codrituzumab (GC33), or a binding fragment of any one thereof). W⁵ may include a reactive chemical functional moiety, non-limiting examples of which are disclosed in the Table E, where W⁵ may be selected to selectively react with W^{5a} in order to provide L⁵ of Formula (III-D).

TABLE E

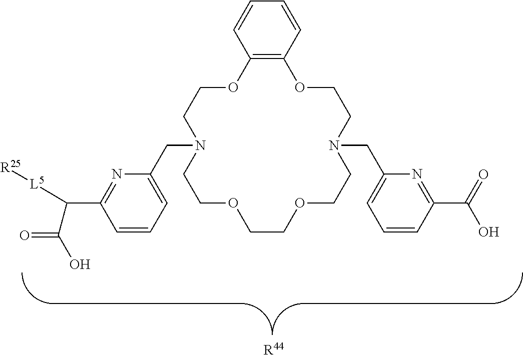

| | | Final Conjugation Product |
|---|---|---|
| R²⁵—W^{5a} | W⁵—R⁴⁴ | (R²⁵—L⁵—R⁴⁴) |
| 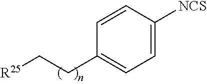 | H₂N—R⁴⁴ | 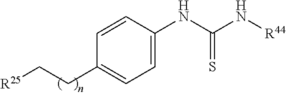 |
| 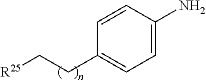 | 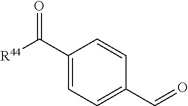 | 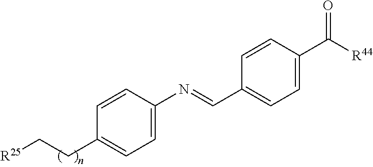 |
| | 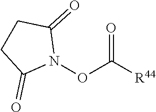 | 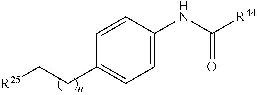 |
| 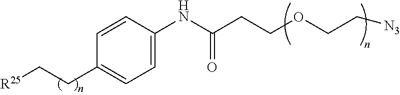 | 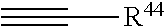 | 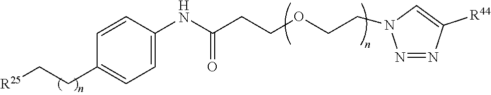 |
| | | and/or |
| | | 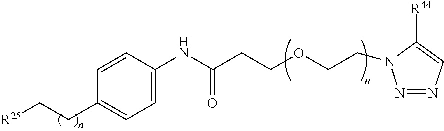 |
| | 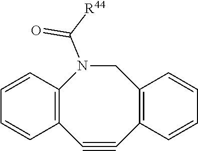 | 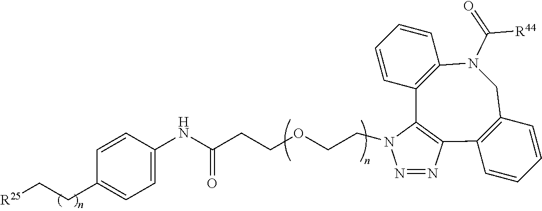 |
| | | and/or |
| | | 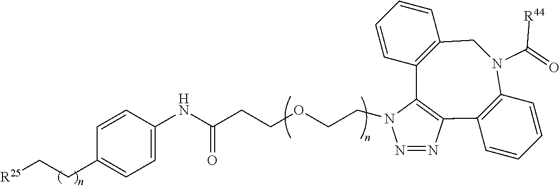 |
| 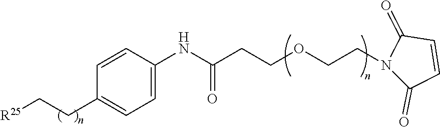 | 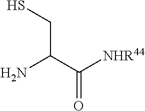 | 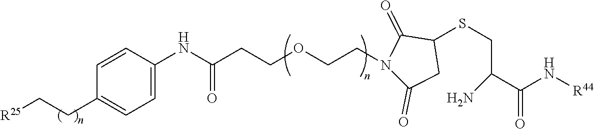 |

In any embodiment herein, it may be that the structures include compounds of Formula (I-E); a modified antibody, modified antibody fragment, or modified binding peptide comprising a linkage arising from conjugation of a compound of Formula (I-E) or pharmaceutically acceptable salt and/or solvate thereof, with an antibody, antibody fragment, or binding peptide; compounds of Formula (II-E); a modified antibody, modified antibody fragment, or modified binding peptide comprising a linkage arising from conjugation of a compound of Formula (II-E) or pharmaceutically acceptable salt and/or solvate thereof, with an antibody, antibody fragment, or binding peptide; and targeting compounds of Formula (III-E):

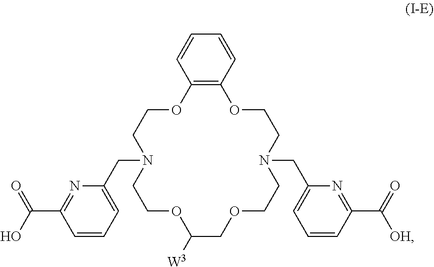

or a pharmaceutically acceptable salt and/or solvate thereof,

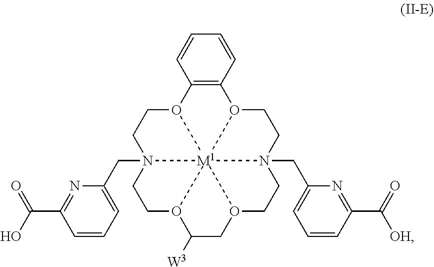

or a pharmaceutically acceptable salt and/or solvate thereof,

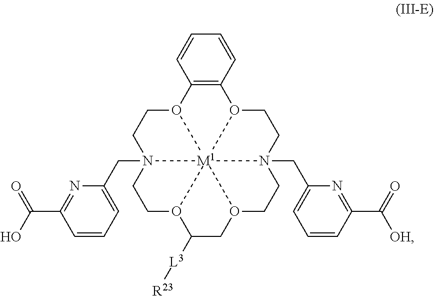

or a pharmaceutically acceptable salt and/or solvate thereof, wherein M¹ is independently at each occurrence a radionuclide.

Targeting compounds of Formula (III-E) may be prepared by a process that includes reacting a compound of Formula (I-E) or (II-E) with R²³—W³a, where Table F provides representative examples (where n is independently at each occurrence 1, 2, 3, 4, 5, 6, 7, 8, 9, or 10). As such, R²³ may be conjugated to macrocycle R⁴⁵ by reaction of complementary chemical functional groups W³ and W^{3a} to form linker L³. For example, R²³—W^{3a} may include a modified target amino acid residue within a protein (e.g., one of the representative antibodies disclosed in Table A or an antigen-binding fragment thereof, a PSMA binding peptide, a somatostatin receptor agonist, a bombesin receptor agonist, a seprase binding compound, or a binding fragment of any one thereof, or an antibody codrituzumab (GC33), or a binding fragment of any one thereof). W³ may include a reactive chemical functional moiety, non-limiting examples of which are disclosed in the Table F, where W³ may be selected to selectively react with W^{3a} in order to provide L³ of Formula (III-E).

TABLE F

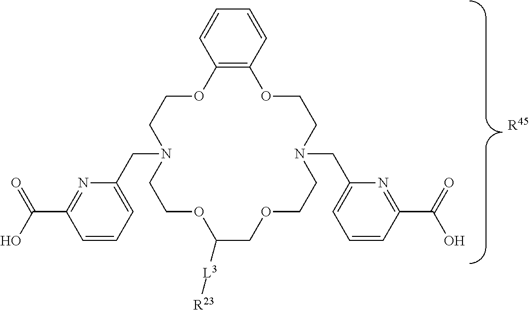

| | | Final Conjugation Product |
|---|---|---|
| R²³—W^{3a} | W³—R⁴⁵ | (R²³—L³—R⁴⁵) |
| 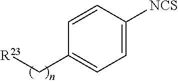 | H₂N—R⁴⁵ | 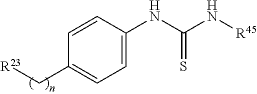 |
| 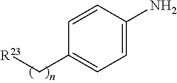 | 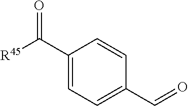 | 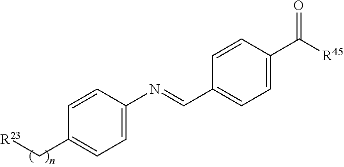 |
| | 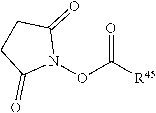 | 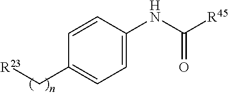 |
| 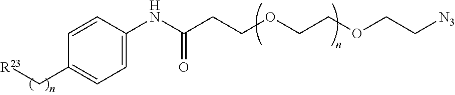 | 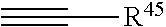 | 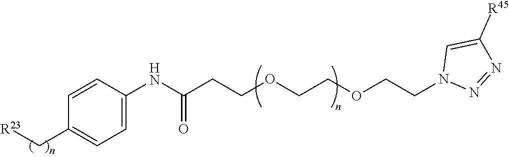 |
| | | and/or |
| | | 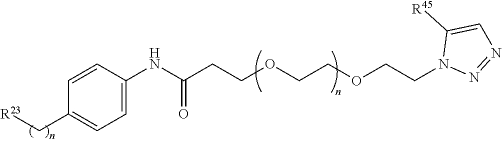 |
| | 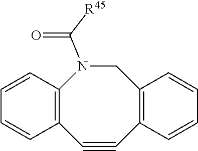 | 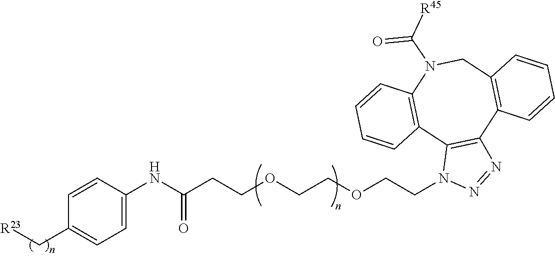 |
| | | and/or |
| | | 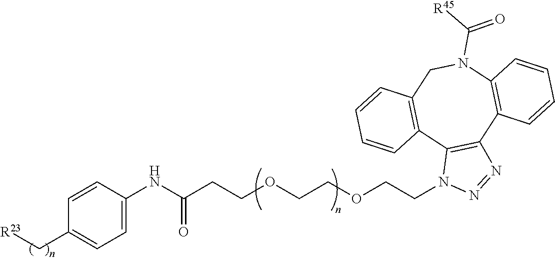 |

In any embodiment herein, it may be that the structures include compounds of Formula (I-F); a modified antibody, modified antibody fragment, or modified binding peptide comprising a linkage arising from conjugation of a compound of Formula (I-F) or pharmaceutically acceptable salt and/or solvate thereof, with an antibody, antibody fragment, or binding peptide; compounds of Formula (II-F); a modified antibody, modified antibody fragment, or modified binding peptide comprising a linkage arising from conjugation of a compound of Formula (II-F) or pharmaceutically acceptable salt and/or solvate thereof, with an antibody, antibody fragment, or binding peptide; and targeting compounds of Formula (III-F)

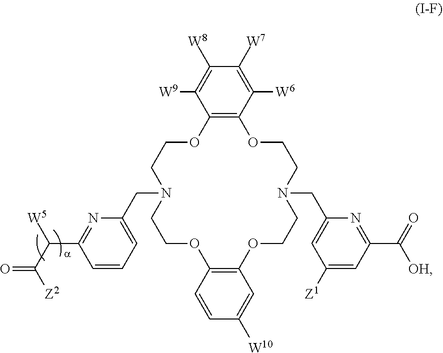

or a pharmaceutically acceptable salt and/or solvate thereof,

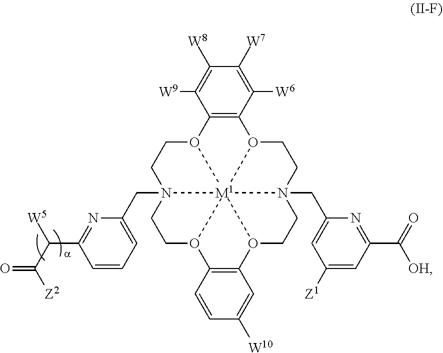

or a pharmaceutically acceptable salt and/or solvate thereof,

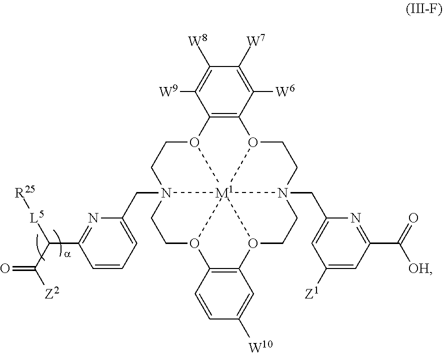

or a pharmaceutically acceptable salt and/or solvate thereof, wherein M¹ is independently at each occurrence a radionuclide.

Targeting compounds of Formula (III-F) may be prepared by a process described herein. For example, in some embodiments, a W⁶, W⁷, W⁸, W⁹ or W¹⁰ moiety of a compound of Formula (III) can be -L⁷-R²⁷, and a targeting compound of Formula (III-F) may be prepared by a process that includes reacting a compound of Formula (I-F) or (II-F) comprising a W⁶, W⁷, W⁸, W⁹ or W¹⁰ moiety with R²⁷—W^{7a}, to form linker L⁷ (e.g., where Table B provides representative examples). In some embodiments, a Z¹ moiety of a compound of Formula (III) can be —X¹-L¹-R²¹, and a targeting compound of Formula (III-F) may be prepared by a process that includes reacting a compound of Formula (I-F) or (II-F) comprising a W¹ moiety with R²¹—W^{1a}, to form linker L¹ (e.g., where Table C provides representative examples). In some other embodiments, a Z² moiety of a compound of Formula (III-F) can be —X²—R²², and a targeting compounds of Formula (III-F) may be prepared by a process that includes reacting a compound of Formula (I-F) or (II-F) comprising a W² moiety with R²²—W^{2a}, to form linker L² (e.g., where Table D provides representative examples). In some other embodiments, a targeting compounds of Formula (III-F) may be prepared by a process that includes reacting a compound of Formula (I-F) or (II-F) comprising a W⁵ moiety with R²⁵—W^{5a}, to form linker L⁵ (e.g., where Table E provides representative examples).

A person of ordinary skill in the art will recognize that numerous chemical conjugation strategies provide ready access to targeting compounds of the present technology, whereby exposed amino acid residues on a protein (e.g., an antibody) undergo well-known reactions with reactive moieties on a prosthetic molecule. For example, amide coupling is a well-known route, where—as an example—lysine residues on the antibody surface react with terminal activated carboxylic acid esters to generate stable amide bonds. Amide coupling is typically mediated by any of several coupling reagents (e.g., HATU, EDC, DCC, HOBT, PyBOP, etc.), which are detailed elsewhere. (See generally Eric Valeur & Mark Bradley, Amide Bond Formation: Beyond the Myth of Coupling Reagents, 38 CHEM. SOC. REv. 606 (2009).) These and other amide coupling strategies are described in a recent review by Tsuchikama. (Kyoji Tsuchikama & Zhiqiang An, Antibody-Drug Conjugates: Recent Advances in Conjugation and Linker Chemistries, 9 PROTEIN CELL 33, 36 (2018); see also, e.g., A. C. Lazar et al., Analysis of the Composition of Immunoconjugates Using Size-Exclusion Chromatography Coupled to Mass Spectrometry, 19 RAPID COMMUN. MASS SPECTROM. 1806 (2005).)

Additionally, a person of ordinary skill in the art will recognize that cysteine coupling reactions may be employed to conjugate prosthetic molecules with thiol-reactive termini to protein surfaces through exposed thiol side chains on cysteine residues on the protein (e.g., antibody) surface. (See generally Tsuchikama & An, supra, at 36-37; see also, e.g., Pierre Adumeau et al., Thiol-Reactive Bifunctional Chelators for the Creation of Site-Selectively Modified Radioimmunoconjugates with Improved Stability, 29 BIOCONJUGATE CHEM. 1364 (2018).) Because cysteine residues readily form disulfide linkages with nearby cysteine residues under physiological conditions, rather than existing as free thiols, some cysteine coupling strategies may rely upon selective reduction of disulfides to generate a higher number of reactive free thiols. (See id.) Cysteine coupling techniques known in the art include, but are not limited to, cys alkylation reactions, cysteine rebridging reactions, and cys-aryl coupling using organometallic palladium reagents. (See, e.g., C. R. Behrens et al., Antibody-Drug Conjugates (ADCs) Derived from Interchain Cysteine Cross-Linking Demonstrates Improved Homogeneity and Other Pharmacological Properties Over Conventional Heterogeneous ADCs, 12 MOL. PHARM. 3986 (2015); Vinogradova et al., Organometallic Palladium Reagents for Cysteine Bioconjugation, 526 NATURE 687 (2015); see also Tsuchikama, supra, at 37 (collecting examples).)

Protein conjugation strategies using non-natural amino acid side chains are also well-known in the art. For example, “click chemistries” provide access to conjugated proteins, by rapid and selective chemical transformations under a diverse range of reaction conditions. Click chemistries are known to yield peptide conjugates with limited by-product formation, despite the presence of unprotected functional groups, in aqueous conditions. One important non-limiting example of a click reaction in the formation of conjugated peptides is the copper(I)-catalyzed azide-alkyne 1,3-dipolar cycloaddition reaction (CuAAC). (See Liyuan Liang & Didier Astruc, The Copper(I)-Catalysed Alkyne-Azide Cycloaddition (CuAAC) “Click” Reaction and Its Applications: An Overview, 255 COORD. CHEM. REV. 2933 (2011); see also, e.g., Herman S. Gill & Jan Marik, Preparation of ¹⁸F-labeled Peptides using the Copper(I)-Catalyzed Azide-Alkyne 1,3-Dipolar Cycloaddition, 6 NATURE PROTOCOLS 1718 (2011).) The CuAAC click reaction may be carried out in the presence of ligands to enhance reaction rates. Such ligands may include, for example, polydentate nitrogen donors, including amines (e.g., tris(triazolyl)methyl amines) and pyridines. (See Liang & Astruc, supra, at 2934 (collecting examples); P. L. Golas et al., 39 MACROMOLECULES 6451 (2006).) Other widely-utilized click reactions include, but are not limited to, thiol-ene, oxime, Diels-Alder, Michael addition, and pyridyl sulfide reactions.

Copper-free (Cu-free) click methods are also known in the art for delivery of therapeutic and/or diagnostic agents, such as radionuclides (e.g., ¹⁸F), chemotherapeutic agents, dyes, contrast agents, fluorescent labels, chemiluminescent labels, or other labels, to protein surfaces. Cu-free click methods may permit stable covalent linkage between target molecules and prosthetic groups. Cu-free click chemistry may include reacting an antibody or antigen-binding fragment, which has been modified with a non-natural amino acid side chain that includes an activating moiety such as a cyclooctyne (e.g., dibenzocyclooctyne (DBCO)), a nitrone or an azide group, with a prosthetic group that presents a corresponding or complementary reactive moiety, such as an azide, nitrone or cyclooctyne (e.g., DBCO). (See, e.g., David. J. Donnelly et al., Synthesis and Biologic Evaluation of a Novel ¹⁸F-Labeled Adnectin as a PET Radioligand for Imaging PD-L1 Expression, 59 J. NUCL. MED. 529 (2018).) For example, where the targeting molecule comprises a cyclooctyne, the prosthetic group may include an azide, nitrone, or similar reactive moiety. Where the targeting molecule includes an azide or nitrone, the prosthetic group may present a complementary cyclooctyne, alkyne, or similar reactive moiety. Cu-free click reactions may be carried out at room temperature, in aqueous solution, in the presence of phosphate-buffered saline (PBS). The prosthetic group may be radiolabeled (e.g., with ¹⁸F) or may be conjugated to any alternative diagnostic and/or therapeutic agent (e.g., a chelating agent). (See id. at 531.)

The compounds of any embodiment and aspect herein of the present technology may be a tripartite compound. Thus, in an aspect, a tripartite compound is provided that includes a first domain that has relatively low but still specific affinity for serum albumin (e.g., 0.5 to 50×10⁻⁶M), a second domain including a chelating moiety such as but not limited to those described herein (e.g., a chelating moiety comprising or arinsing from a compound of Formula (I) or Formula (II)), and a third domain that includes tumor targeting moiety (TTT) having relatively high affinity for a tumor antigen (e.g., 0.5 to 50×10⁻⁹M). The following exemplary peptide receptors, enzymes, cell adhesion molecules, tumor associated antigens, growth factor receptors and cluster of differentiation antigens are useful targets for constructing the TTT domain: glypican-3 (GPC3) receptor, somatostatin peptide receptor-2 (SSTR2), gastrin-releasing peptide receptor, seprase (FAP-alpha), incretin receptors, glucose-dependent insulinotropic polypeptide receptors, VIP-1, NPY, folate receptor, LHRH, and αvβ3, an overexpressed peptide receptor, a neuronal transporter (e.g., noradrenaline transporter (NET)), or other tumor associated proteins such as EGFR, HER-2, VGFR, MUC-1, CEA, MUC-4, ED2, TF-antigen, endothelial specific markers, neuropeptide Y, uPAR, TAG-72, CCK analogs, VIP, bombesin, VEGFR, tumor-specific cell surface proteins, GLP-1, CXCR4, Hepsin, TMPRSS2, caspaces, Alpha V beta six, cMET. Other such targets will be apparent to those of skill in the art, and compounds that bind these can be incorporated in the TTT to produce a tripartite radiotherapeutic compound.

The following Formulas (L)-(LIV) provide exemplary general structures for tripartite compounds of the present technology.

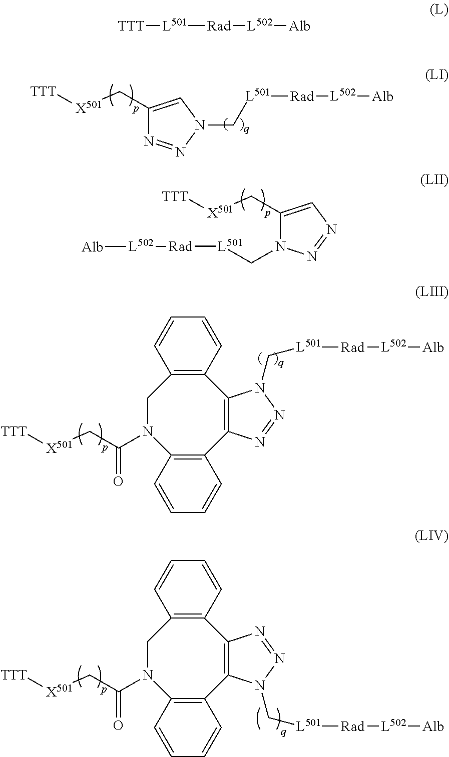

where
   TTT is independently at each occurrence a binding domain for a glypican-3 (GPC3) receptor, a somatostatin peptide receptor-2 (SSTR2), a gastrin-releasing peptide receptor, a seprase (FAP-alpha), an incretin receptor, a glucose-dependent insulinotropic polypeptide receptor, VIP-1, NPY, a folate receptor, LHRH, αvβ3, an overexpressed peptide receptor, a neuronal transporter (e.g., noradrenaline transporter (NET)), a receptor for a tumor associated protein (such as EGFR, HER-2, VGFR, MUC-1, CEA, MUC-4, ED2, TF-antigen, endothelial specific markers, neuropeptide Y, uPAR, TAG-72, CCK analogs, VIP, bombesin, VEGFR, tumor-specific cell surface proteins, GLP-1, CXCR4, Hepsin, TMPRSS2, caspaces, Alpha V beta six, cMET, or combination of any two or more thereof), or a cominbation of any two or more thereof,
   X⁵⁰¹ is independently at each occurrence absent, O, S, or NH;
   L⁵⁰¹ is independently at each occurrence absent, —C(O)—, —C(O)—NR⁴—, —C(O)—NR⁵—C₁-C₁₂ alkylene-, —C₁-C₁₂ alkylene-C(O)—, —C(O)—NR⁶—C₁-C₁₂ alkylene-C(O)—, -arylene-, —O(CH₂CH₂O)ᵣ—CH₂CH₂C(O)—, —O(CH₂CH₂O)ᵣᵣ—CH₂CH₂C(O)—NH—, —O(CH₂CH₂O)ᵣᵣᵣ—CH₂CH₂—, an amino acid, a peptide of 2, 3, 4, 5, 6, 7, 8, 9, or 10 amino acids, or a combination of any two or more thereof, where r is 0, 1, 2, 3, 4, 5, 6, 7, 8, or 9, rr is 0, 1, 2, 3, 4, 5, 6, 7, 8, or 9, rrr is 0, 1, 2, 3, 4, 5, 6, 7, 8, or 9, and where R⁴, R⁵, and R⁶ are each independently H, alkyl, or aryl;
Rad is independently at each occurrence a moiety capable of including a radionuclide, optionally further including a radionuclide;
L⁵⁰² is independently at each occurrence absent, —C(O)—, —(CH₂CH₂O)ₛ—CH₂CH₂C(O)—, —(CH₂CH₂O)ₛₛ—CH₂CH₂C(O)—NH—, —(CH₂CH₂O)ₛₛₛ—CH₂CH₂—, an amino acid, —CH(CO₂H)—(CH₂)₄—, —CH(CO₂H)—(CH₂)₄—NH—, a peptide of 2, 3, 4, 5, 6, 7, 8, 9, 10, 11, 12, 13, 14, 15, 16, 17, 18, 19, or 20 amino acids, or a combination of any two or more thereof, where s is 0, 1, 2, 3, 4, 5, 6, 7, 8, 9, 10, 11, 12, 13, 14, 15 , 16, 17, 18, or 19, ss is 0, 1, 2, 3, 4, 5, 6, 7, 8, 9, 10, 11, 12, 13, 14, 15, 16, 17, 18, or 19, and sss is 0, 1, 2, 3, 4, 5, 6, 7, 8, 9, 10, 11, 12, 13, 14, 15, 16, 17, 18, or 19;
Alb is independently at each occurrence an albumin-binding moiety;
p is independently at each occurrence 0, 1, 2, or 3; and
q is independently at each occurrence 1 or 2.

In any embodiment disclosed herein, the radionuclide may be ¹⁷⁷Lu³⁺, ¹⁷⁵Lu³, ⁴⁵Sc³⁺, ⁶⁶Ga³⁺, ⁶⁷Ga³⁺, ⁶⁸Ga³⁺, ⁶⁹Ga³⁺, ⁷¹Ga³⁺, ⁸⁹Y³⁺, ⁸⁶Y³⁺, ⁸⁹Zr⁴⁺, ⁹⁰Y³⁺, , ^{99m}Tc⁺¹, ¹¹¹In³⁺, ¹¹³In³, ¹¹⁵In³⁺, ¹³⁹La³⁺, ¹³⁶Ce³⁺, ¹³⁸Te³⁺, ¹⁴⁰Ce³⁺, ¹⁴²Ce³⁺, ¹⁵¹Eu³⁺, ¹⁵³Eu³⁺, , ¹⁵²DY³⁺, ¹⁴⁹Tb³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, ¹⁶⁰Gd³⁺, ¹⁸⁸Re⁺¹, ¹⁸⁶Re⁺¹, ²¹³Bi³⁺, ²¹¹At⁺, ²¹⁷At⁺227Th⁴⁺²²⁶Th⁴⁺, ²²⁵Ac³⁺, ²³³Ra²⁺ ¹⁵²DY³⁺, ²¹³Bi³⁺, ²¹²Bi³⁺, ²¹¹Bi³⁺, ²¹²Pb²⁺, ²¹²Pb⁴⁺, ²⁵⁵Fm³⁺, or uranium-230. For example, the radionuclide may be an alpha-emitting radionuclide such as ²¹³Bi³⁺, ²¹¹At⁺, ²²⁵Ac³⁺, ¹⁵²DY³⁺, ²¹²Bi³⁺, ²¹¹Bi³⁺, ²¹⁷At⁺, ²²⁷Th⁴⁺, ²²⁶Th⁴⁺, ²³³Ra²⁺, ²¹²Pb²⁺, or ²¹²Pb⁴⁺.

In any embodiment disclosed herein, it may be the tripartite compounds of Formulas (L)-(LIV) are of Formulas (LV)-(LIX)

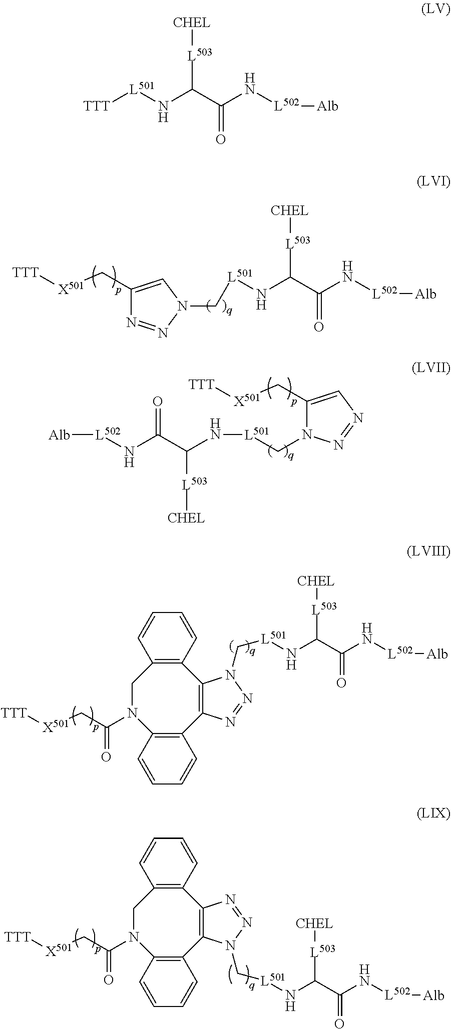

where L⁵⁰³ is independently at each occurrence absent, —C(O)—, —C₁-C₁₂ alkylene-, —C₁-C₁₂ alkylene-C(O)—, —C₁-C₁₂ alkylene-NR¹⁰—, -arylene-, —(CH₂CH₂O)_{z}—CH₂CH₂C(O)—, —(CH₂CH₂O)_{zz}—CH₂CH₂C(O)—NH—, —(CH₂CH₂O)_{zzz}—CH₂CH₂—, an amino acid, —CH(CO₂H)—(CH₂)₄—, —CH(CO₂H)—(CH₂)₄—NH—, a peptide of 2, 3, 4, 5, 6, 7, 8, 9, 10, 11, 12, 13, 14, 15, 16, 17, 18, 19, or 20 amino acids, or a combination of any two or more thereof, where z is 0, 1, 2, 3, 4, 5, 6, 7, 8, 9, 10, 11, 12, 13, 14, 15, 16, 17, 18, or 19, zz is 0, 1, 2, 3, 4, 5, 6, 7, 8, 9, 10, 11, 12, 13, 14, 15, 16, 17, 18, or 19, and zzz is 0, 1, 2, 3, 4, 5, 6, 7, 8, 9, 10, 11, 12, 13, 14, 15, 16, 17, 18, or 19; and CHEL is independently at each occurrence a covalently conjugated chelator that optionally includes a chelated radionuclide (e.g., according to Formula (I), (II), or (III) of the present technology).

The albumin-binding moiety plays a role in modulating the rate of blood plasma clearance of the compounds in a subject, thereby increasing circulation time and compartmentalizing the cytotoxic action of cytotoxin-containing domain and/or imaging capability of the imaging agent-containing domain in the plasma space instead of normal organs and tissues that may express antigen. Without being bound by theory, this component of the structure is believed to interact reversibly with serum proteins, such as albumin and/or cellular elements. The affinity of this albumin-binding moiety for plasma or cellular components of the blood may be configured to affect the residence time of the compounds in the blood pool of a subject. In any embodiment herein, the albumin binding-moiety may be configured so that it binds reversibly or non-reversibly with albumin when in blood plasma. In any embodiment herein, the albumin binding-moiety may be selected such that the binding affinity of the compound with human serum albumin is about 5 μM to about 15 μM.

By way of example, the albumin-binding moiety of any embodiment herein may include a short-chain fatty acid, medium-chain chain fatty acid, a long-chain fatty acid, myristic acid, a substituted or unsubstituted indole-2-carboxylic acid, a substituted or unsubstituted 4-oxo-4-(5,6,7,8-tetrahydronaphthalen-2-yl)butanoic acid, a substituted or unsubstituted naphthalene acylsulfonamide, a substituted or unsubstituted diphenylcyclohexanol phosphate ester, a substituted or unsubstituted 2-(4-iodophenyl)acetic acid, a substituted or unsubstituted 3-(4-iodophenyl)propionic acid, or a substituted or unsubstituted 4-(4-iodophenyl)butanoic acid. Certain representative examples of albumin-binding moieties that may be included in any embodiment herein include one or more of the following.

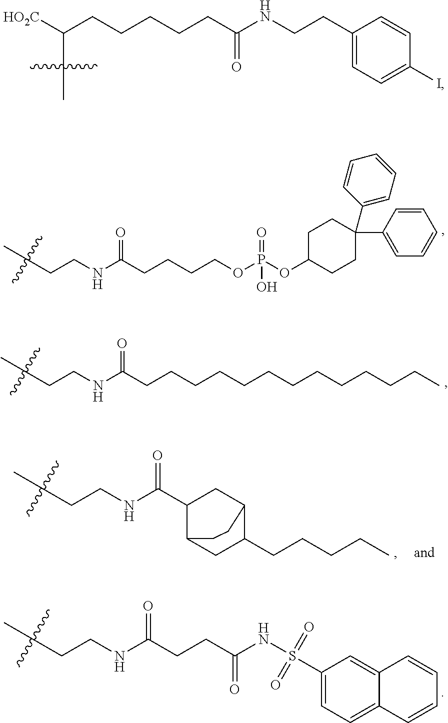 [SMILES: *C(CCCCCC(=O)NCCc1ccc(I)cc1)C(=O)O ; *CCNC(=O)CCCCOP(=O)(O)OC1CCC(c2ccccc2)(c2ccccc2)CC1 ; *CCNC(=O)CCCCCCCCCCCCC ; *CCNC(=O)C1CC2CCC1CC2CCCCC ; *CCNC(=O)CCC(=O)NS(=O)(=O)c1ccc2ccccc2c1]

In any embodiment herein, the tripartite compounds may include an albumin-binding moiety that is

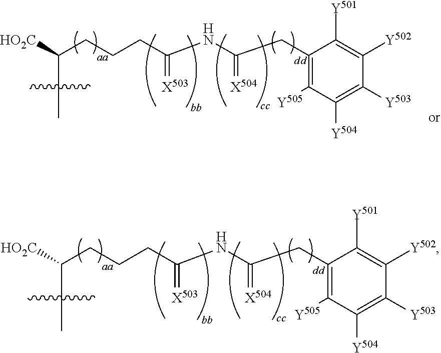

where Y⁵⁰¹, Y⁵⁰², Y⁵⁰³, Y⁵⁰⁴, and Y⁵⁰⁵ are independently H, halo, or alkyl, X⁵⁰³, X⁵⁰⁴, X⁵⁰⁵, and X⁵⁰⁶ are each independently O or S, aa is independently at each occurrence 0, 1, or 2, bb is independently at each occurrence 0 or 1, cc is independently at each occurrence 0 or 1, and dd is independently at each occurrence 0, 1, 2, 3, or 4. In any embodiment herein, it may be that bb and cc cannot be the same value. In any embodiment herein, it may be that Y⁵⁰³ is I and each of Y⁵⁰¹, Y⁵⁰², Y⁵⁰³, Y⁵⁰⁴, and Y⁵⁰⁵ are each independently H.

Representative chelators useful in any embodiment of the present technology include, but are not limited to, a covalently conjugated substituted or unsubstituted chelator of the following group:
6,6′-((18-isothiocyanato-2,3,5,6,8,9,11,12,14,15-decahydro-4H,13H-benzo[b][1,4,10,13]tetraoxa[7,16]diazacyclooctadecine-4,13-diyl)bis(methylene))dipicolinic acid (H2BZmacropa-NCS);
6,6′-((2,3,5,6,8,9,11,12,14,15-decahydro-4H,13H-benzo[b][1,4,10,13]tetraoxa[7,16]diazacyclooctadecine-4,13-diyl)bis(methylene))dipicolinic acid (H2BZmacropa); and
6,6′-((6,7,9,10,17,18,20,21-octahydro-8H,19H-dibenzo[b,k][1,4,10,13]tetraoxa[7,16]diazacyclooctadecine-8,19-diyl)bis(methylene))dipicolinic acid (H2BZ2macropa).

It is to be understood that a “covalently conjugated” chelator means a chelator (such as those listed above) wherein one or more bonds to a hydrogen atom contained therein are replaced by a bond to an atom of the remainder of the Rad and/or CHEL moiety, to L⁵⁰¹, and/or to L⁵⁰², or a pi bond between two atoms is replaced by a bond from one of the two atoms to an atom of the remainder of the Rad and/or CHEL moiety, to L⁵⁰¹, and/or to L⁵⁰², and the other of the two atoms includes a new bond, e.g. to a hydrogen (such as reaction of an —NCS group in the chelator to provide the covalently conjugated chelator).

In any embodiment disclosed herein, it may be that the CHEL of the tripartite compounds is a chelator as provided in the compounds of Formula (I), (II), or (III). For example, tripartite compound may be a targeting compound of Formula (III) where R²¹, R²², R²³, R²⁴, R²⁵, and R²⁷ are each independently

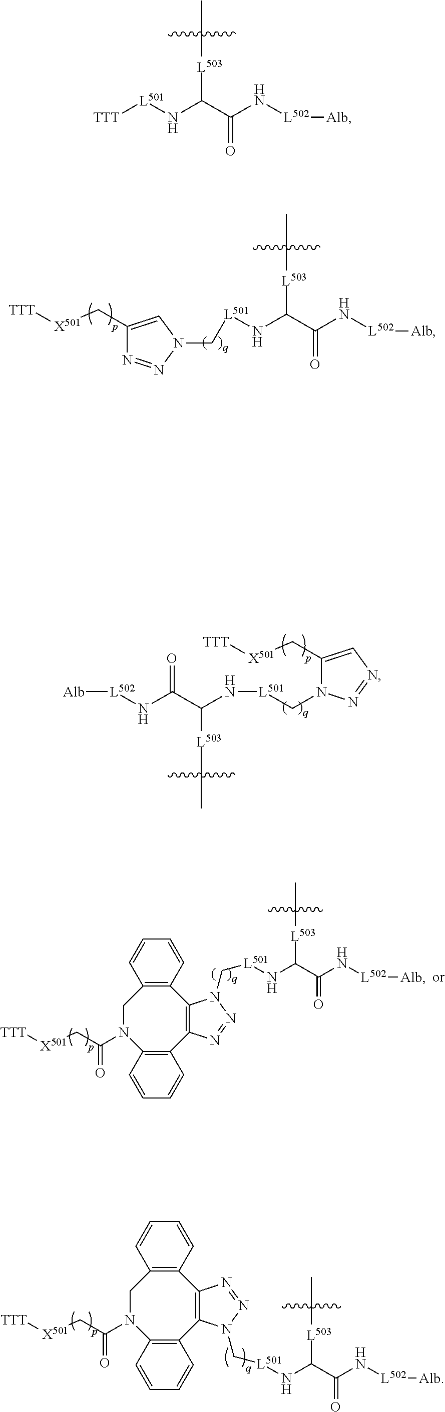

In any embodiment disclosed herein, TTT may be an antibody. In embodiments, TTT may be codrituzumab (GC33), or a binding fragment thereof. In embodiments, TTT may be a targeting moiety that targets the glypican-3 (GPC3) receptor. In embodiments, TTT comprises a targeting moiety that targets the glypican-3 (GPC3) receptor.

In any embodiment, TTT may be

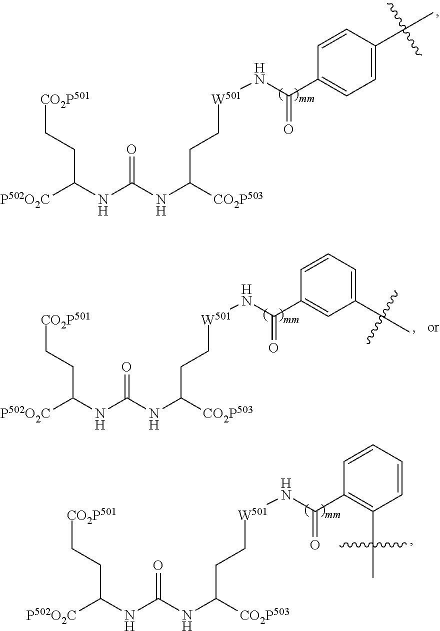

where
W⁵⁰¹ is —C(O)—, —(CH₂)_{ww}—, or —(CH₂)ₒₒ—NH₂—C(O)—;
mm is 0 m or 1;
ww is 1 or 2;
oo is 1 or 2; and
P⁵⁰¹ P⁵⁰² and P⁵⁰³ are each independently H, methyl, benzyl, 4-methoxybenzyl, or tert-butyl.

In any embodiment herein, it may be that each of p⁵⁰¹, p⁵⁰², and P⁵⁰³ are H.

In some embodiments, the tripartite compounds (e.g., the tumor targeting domain) of the present technology comprise a moiety with one of the following structures, which can, e.g., target seprase (Fibroblast Activation Protein/FA-P):

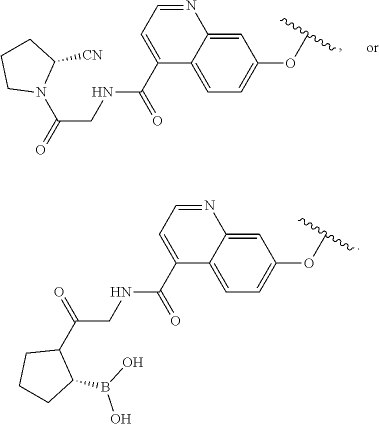 [SMILES: *Oc1ccc2c(C(=O)NCC(=O)N3CCC[C@@H]3C#N)ccnc2c1 ; *Oc1ccc2c(C(=O)NCC(=O)C3CCC[C@H]3B(O)O)ccnc2c1]

It is to be understood that the tripartite compounds of the present technology can include variations on any of the three domains: e.g., the domain including the chelator, the domain including the albumin-binding group, or the domain including the tumor targeting moiety.

The present technology also provides compositions (e.g., pharmaceutical compositions) and medicaments comprising any of one of the embodiments of the compounds of Formulas (I), (II), (III), any one of the modified antibodies, modified antibody fragments, or modified binding peptides of the present technology disclosed herein, or any one of the embodiments of the tripartite compounds disclosed herein and a pharmaceutically acceptable carrier or one or more excipients or fillers (collectively referred to as “pharmaceutically acceptable carrier” unless otherwise specified). The compositions may be used in the methods and treatments described herein. The pharmaceutical composition may include an effective amount of any embodiment of the compounds of the present technology for treating the cancer (e.g., liver cancer) and/or mammalian tissue overexpressing e.g., a glypican-3 (GPC3) receptor and/or PSMA; or an effective amount of any embodiment of the modified antibody, modified antibody fragment, or modified binding peptide of the present technology for treating the cancer (e.g., liver cancer) and/or mammalian tissue overexpressing e.g., a glypican-3 (GPC3) receptor and/or PSMA; or an effective amount of any embodiment of the tripartite compound of the present technology for treating the cancer (e.g., liver cancer) and/or mammalian tissue overexpressing e.g., a glypican-3 (GPC3) receptor and/or PSMA. In an related aspect, a method of treating a subject is provided, wherein the method includes administering a targeting compound of the present technology to the subject or administering a modified antibody, modified antibody fragment, or modified binding peptide of the present technology to the subject. In any embodiment disclosed herein, it may be that the subject suffers from cancer (e.g., liver cancer) and/or mammalian tissue overexpressing e.g., a glypican-3 (GPC3) receptor and/or PSMA. In any embodiment herein, it may be the administering includes administering an effective amount of any embodiment of the compounds of the present technology for treating the cancer (e.g., liver cancer) and/or mammalian tissue overexpressing, e.g., a glypican-3 (GPC3) receptor and/or PSMA, of the compound or an effective amount of any embodiment of the modified antibody, modified antibody fragment, or modified binding peptide of the present technology for treating the cancer (e.g., liver cancer) and/or mammalian tissue overexpressing, e.g., a glypican-3 (GPC3) receptor and/or PSMA, or an effective amount of any embodiment of the tripartite compound of the present technology for treating the cancer (e.g., liver cancer) and/or mammalian tissue overexpressing e.g., a glypican-3 (GPC3) receptor and/or PSMA. The subject may suffer from a mammalian tissue expressing a somatostatin receptor, a bombesin receptor, seprase, or a combination of any two or more thereof and/or mammalian tissue overexpressing PSMA. The mammalian tissue of any embodiment disclosed herein may include one or more of a growth hormone producing tumor, a neuroendocrine tumor, a pituitary tumor, a vasoactive intestinal peptide-secreting tumor, a small cell carcinoma of the lung, gastric cancer tissue, pancreatic cancer tissue, a neuroblastoma, and a metastatic cancer. In any embodiment disclosed herein, the subject may suffer from one or more of a liver cancer, a glioma, a breast cancer, an adrenal cortical cancer, a cervical carcinoma, a vulvar carcinoma, an endometrial carcinoma, a primary ovarian carcinoma, a metastatic ovarian carcinoma, a non-small cell lung cancer, a small cell lung cancer, a bladder cancer, a colon cancer, a primary gastric adenocarcinoma, a primary colorectal adenocarcinoma, a renal cell carcinoma, and a prostate cancer. In any embodiment disclosed herein, the composition (e.g., pharmaceutical composition) and/or medicament may be formulated for parenteral administration. In any embodiment disclosed herein, the composition (e.g., pharmaceutical composition) and/or medicament may be formulated for intraveneous administration. In any embodiment disclosed herein, the administering step of the method may include parenteral administration. In any embodiment disclosed herein, the administering step of the method may include intraveneous administration.

In any embodiment herein, the effective amount may be determined in relation to a subject. “Effective amount” refers to the amount of a compound or composition required to produce a desired effect. One non-limiting example of an effective amount includes amounts or dosages that yield acceptable toxicity and bioavailability levels for therapeutic (pharmaceutical) use including, but not limited to, the treatment of e.g., one or more of a glioma, a liver cancer, a breast cancer, an adrenal cortical cancer, a cervical carcinoma, a vulvar carcinoma, an endometrial carcinoma, a primary ovarian carcinoma, a metastatic ovarian carcinoma, a non-small cell lung cancer, a small cell lung cancer, a bladder cancer, a colon cancer, a primary gastric adenocarcinoma, a primary colorectal adenocarcinoma, a renal cell carcinoma, and a prostate cancer. Another example of an effective amount includes amounts or dosages that are capable of reducing symptoms associated with e.g., one or more of a glioma, a liver cancer, a breast cancer, an adrenal cortical cancer, a cervical carcinoma, a vulvar carcinoma, an endometrial carcinoma, a primary ovarian carcinoma, a metastatic ovarian carcinoma, a non-small cell lung cancer, a small cell lung cancer, a bladder cancer, a colon cancer, a primary gastric adenocarcinoma, a primary colorectal adenocarcinoma, a renal cell carcinoma, and a prostate cancer, such as, for example, reduction in proliferation and/or metastasis of liver cancer, prostate cancer, breast cancer, or bladder cancer. The effective amount may be from about 0.01 g to about 1 mg of the compound per gram of the composition, and preferably from about 0.1 g to about 500 μg of the compound per gram of the composition. As used herein, a “subject” or “patient” is a mammal, such as a cat, dog, rodent or primate. Typically the subject is a human, and, preferably, a human suffering from or suspected of suffering from one or more of a glioma, a liver cancer, a breast cancer, an adrenal cortical cancer, a cervical carcinoma, a vulvar carcinoma, an endometrial carcinoma, a primary ovarian carcinoma, a metastatic ovarian carcinoma, a non-small cell lung cancer, a small cell lung cancer, a bladder cancer, a colon cancer (such as colon adenocarcinoma), a primary gastric adenocarcinoma, a primary colorectal adenocarcinoma, a renal cell carcinoma, and a prostate cancer. The term “subject” and “patient” can be used interchangeably.

In any of the embodiments of the present technology described herein, the pharmaceutical composition may be packaged in unit dosage form. The unit dosage form is effective in treating one or more of a liver cancer, a glioma, a breast cancer, an adrenal cortical cancer, a cervical carcinoma, a vulvar carcinoma, an endometrial carcinoma, a primary ovarian carcinoma, a metastatic ovarian carcinoma, a non-small cell lung cancer, a small cell lung cancer, a bladder cancer, a colon cancer (such as colon adenocarcinoma), a primary gastric adenocarcinoma, a primary colorectal adenocarcinoma, a renal cell carcinoma, and a prostate cancer. Generally, a unit dosage including a compound of the present technology will vary depending on patient considerations. Such considerations include, for example, age, protocol, condition, sex, extent of disease, contraindications, concomitant therapies and the like. An exemplary unit dosage based on these considerations may also be adjusted or modified by a physician skilled in the art. For example, a unit dosage for a patient comprising a compound of the present technology may vary from 1×10⁻⁴ g/kg to 1 g/kg, preferably, 1×10⁻³ g/kg to 1.0 g/kg. Dosage of a compound of the present technology may also vary from 0.01 mg/kg to 100 mg/kg or, preferably, from 0.1 mg/kg to 10 mg/kg. Suitable unit dosage forms, include, but are not limited to powders, tablets, pills, capsules, lozenges. suppositories. patches. nasal sprays, injectibles, implantable sustained-release formulations, rnucoadherent films, topical varnishes, lipid complexes, etc.

The pharmaceutical compositions may be prepared by mixing one or more of the compounds of Formulas (I), (II), (III), or any one of the modified antibodies, modified antibody fragments, or modified binding peptides of the present technology, or any embodiment of the tripartite compound of the present technology, pharmaceutically acceptable salts thereof, stereoisomers thereof, tautomers thereof, or solvates thereof, with pharmaceutically acceptable carriers, excipients, binders, diluents or the like to prevent and treat disorders associated with cancer (e.g., liver cancer) and/or a mammalian tissue overexpressing glypican-3 (GPC3) receptor and/or a mammalian tissue overexpressing PSMA. The compounds and compositions described herein may be used to prepare formulations and medicaments that treat e.g., liver cancer, prostate cancer, breast cancer, or bladder cancer. Such compositions may be in the form of, for example, granules, powders, tablets, capsules, syrup, suppositories, injections, emulsions, elixirs, suspensions or solutions. The instant compositions may be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference. The instant compositions may also include, for example, micelles or liposomes, or some other encapsulated form.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Various assays and model systems can be readily employed to determine the therapeutic effectiveness of the treatment according to the present technology.

For the indicated condition, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

In another aspect, the present technology provides a method of treating cancer (e.g., liver cancer) by administering an effective amount of the targeting composition according to Formula (III) to a subject having cancer. Since a cancer cell targeting agent can be selected to target any of a wide variety of cancers, the cancer considered herein for treatment is not limited. The cancer can be essentially any type of cancer. For example, antibodies or peptide vectors can be produced to target any of a wide variety of cancers. The targeting compositions described herein are typically administered by injection into the bloodstream, but other modes of administration, such as oral or topical administration, are also considered. In some embodiments, the targeting composition may be administered locally, at the site where the target cells are present, i.e., in a specific tissue, organ, or fluid (e.g., blood, cerebrospinal fluid, etc.). Any cancer that can be targeted through the bloodstream is of particular consideration herein. Some examples of applicable body parts containing cancer cells include the breasts, lungs, stomach, intestines, prostate, ovaries, cervix, pancreas, kidney, liver, skin, lymphs, bones, bladder, uterus, colon, rectum, and brain. The cancer can also include the presence of one or more carcinomas, sarcomas, lymphomas, blastomas, or teratomas (germ cell tumors). The cancer may also be a form of leukemia. In some embodiments, the cancer is a triple negative breast cancer. In some embodiments, the cancer is a liver cancer.

As is well known in the art, the dosage of the active ingredient(s) generally depends on the disorder or condition being treated, the extent of the disorder or condition, the method of administration, size of the patient, and potential side effects. In different embodiments, depending on these and other factors, a suitable dosage of the targeting composition may be precisely, at least, above, up to, or less than, for example, 1 mg, 10 mg, 50 mg, 100 mg, 200 mg, 300 mg, 400 mg, 500 mg, 600 mg, 700 mg, 800 mg, 900 mg, 1000 mg, 1200 mg, or 1500 mg, or a dosage within a range bounded by any of the foregoing exemplary dosages. Furthermore, the composition can be administered in the indicated amount by any suitable schedule, e.g., once, twice, or three times a day or on alternate days for a total treatment time of one, two, three, four, or five days, or one, two, three, or four weeks, or one, two, three, four, five, or six months, or within a time frame therebetween. Alternatively, or in addition, the composition can be administered until a desired change in the disorder or condition is realized, or when a preventative effect is believed to be provided.

The examples herein are provided to illustrate advantages of the present technology and to further assist a person of ordinary skill in the art with preparing or using the compounds of the present technology or salts, pharmaceutical compositions, derivatives, prodrugs, or tautomeric forms thereof. The examples herein are also presented in order to more fully illustrate the preferred aspects of the present technology. The examples should in no way be construed as limiting the scope of the present technology, as defined by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### EXAMPLES

### Example 1: Exemplary Synthetic Procedures and Characterization of the Compounds

Materials and Instrumentation. All reagents and solvents were obtained commercially and used without further purification unless otherwise noted. Deionized water (>18 MΩ·cm) was obtained from an Elga Purelab Flex 2 water purification system. Organic solvents were of ACS grade or higher. Solvents noted as dry were obtained following storage over activated 3 Å molecular sieves.

NMR spectra were recorded at 25° C. on a 500 MHz Bruker AVIII HD spectrometer equipped with a broadband Prodigy cryoprobe. Chemical shifts are reported in parts per million (ppm). ¹H NMR and ¹³C{¹H} NMR peaks were referenced to TMS internal standard or to the residual solvent signal (spectra acquired in D₂O were spiked MeCN as an internal reference). ¹⁹F NMR spectra were referenced to an internal standard of fluorobenzene.

High-resolution mass spectra (HRMS) were acquired on an Exactive Orbitrap mass spectrometer (Thermo Scientific, Waltham, MA) in positive electrospray ionization (ESI) mode or direct analysis in real time (DART) mode. UV-vis spectra were recorded on a Shimadzu UV-1900 spectrometer (Shimadzu, Kyoto, Japan) fitted with a temperature-controlled circulating water bath and a stirrer. Elemental analyses were performed by Atlantic Microlab, Inc. (Norcross, GA).

High-performance liquid chromatography (HPLC) was performed using a LC-20AP (preparative) or a LC-20AT (analytical) pump coupled to an SPD-20AV UV-vis detector monitoring at 270 nm (Shimadzu, Japan). Analytical HPLC was carried out using an Ultra Aqueous C18 column, 100 Å, 5 m, 250 mm×4.6 mm (Restek, Bellefonte, PA) at a flow rate of 1.0 mL/min. Semipreparative purification was performed using an Epic Polar preparative column, 120 Å, 10 m, 25 cm×20 mm (ES Industries, West Berlin, NJ) at a flow rate of 14 mL/min.
All HPLC methods employed a binary mobile phase (A+B or A+C).
Solvent A; 0.1% trifluoroacetic acid (TFA) in H₂O
Solvent B; 0.1% TFA in MeOH
Solvent C; 0.1% TFA in MeCN.
The following linear-gradient HPLC methods were employed:
Method quick-MeCN (solvents A/C)→10% C (0-5 min), 10-100% C (5-20 min)
Method prep-MeCN (solvents A/C)→10% C (0-5 min), 10-100% C (5-25 min)
Method prep-MeOH (solvents A/B)→10% B (0-5 min), 10-100% B (5-25 min)
Method prep-MeOH_slow (solvents A/B)→5% B (0-10 min), 5-100% B (10-32 min)

Synthesis and Characterization of H₂BZmacropa. H₂BZmacropa was prepared according to below procedure.

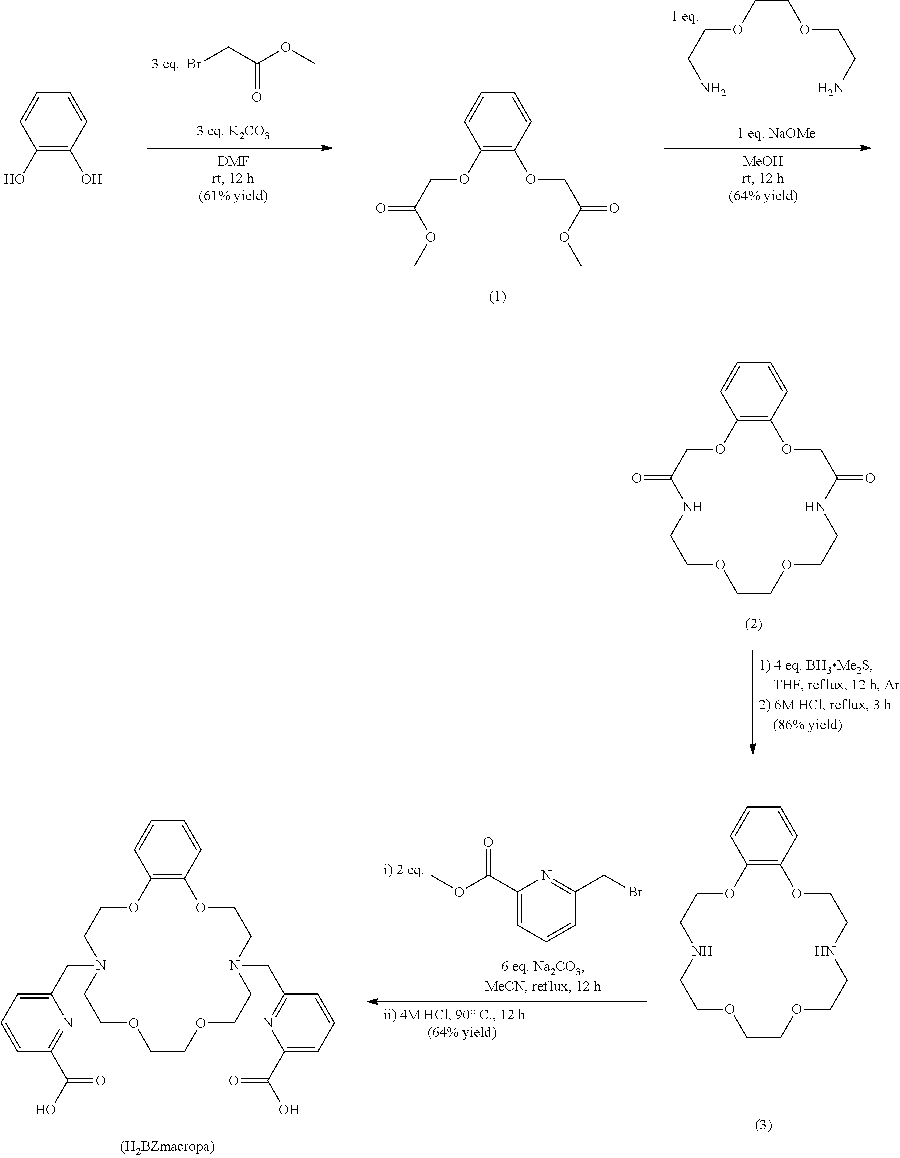

### Benzene-1,2-dioxyacetic acid dimethyl ester (1)

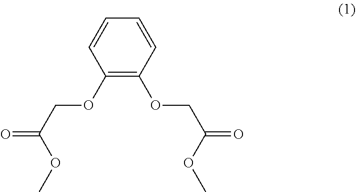

Catechol (5 g, 1 eq., 45.41 mmol), methyl 2-bromoacetate (20.840 g, 3 eq., 136.23 mmol), and K₂CO₃ (20 g, 3.2 eq., 145.31 mmol) were mixed in DMF (25 mL) and stirred overnight at room temperature. The reaction mixture was poured into diethyl ether (150 mL) and the organic phase was washed with water (5×50 mL). The organic layer was collected and dried over anhydrous Na₂SO₄. After filtration, the dried organic layer was concentrated to about 75 mL and cooled in a refrigerator to induce crystallization. The crystals were collected by filtration and dried under vacuum to yield the product as a white solid (7.1 g, 61% yield). ¹H NMR (500 MHz, CDCl₃) δ 6.96 (m, 1H, Ar—CH), 6.89 (m, 1H, Ar—CH), 4.73 (s, 2H, CH₂), 3.79 (s, 3H, CH₃). ¹³C{¹H} NMR (126 MHz, CDCl₃) δ 169.60, 148.12, 122.79, 115.59, 66.74, 52.32. ESI-HRMS: m/z=277.069 ([M+Na]⁺, Caled; 277.068).

### 5,6,8,9,12,13-Hexahydro-2H,11H-1,7,10,16,4,13-benzotetraoxadiazacyclooctadecine-3,14(4H,15H)-dione (2)

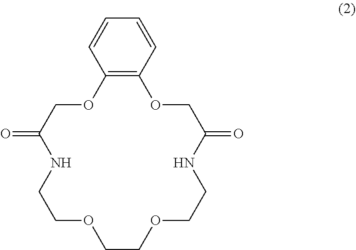 [SMILES: CN1CCOCCOCCNC(=O)COc2ccccc2OCC1=O]

A homogenous solution of 1 (5.085 g, 1 eq., 20 mmol), 2,2′-(ethylenedioxy)diethylamine (2.964 g, 1 eq., 20 mmol), and sodium methoxide (1.080 g, 1 eq., 20 mmol) in MeOH (200 mL) was stirred at room temperature overnight. A lot of precipitate formed during the course of the reaction. The reaction mixture was cooled in a refrigerator for 3 h and the precipitate was collected via filtration while cold. Another batch of precipitate was obtained by concentrating the filtrate to 100 mL and cooling it in a refrigerator overnight. The collected precipitate was washed with cold EtOH (20 mL) and dried under vacuum to yield the product as a white solid (4.324 g, 64% yield). ¹H NMR (500 MHz, CDCl₃) δ 7.12 (s, 1H, NH), 6.98 (m, 1H, Ar—H), 6.91 (m, 1H, Ar—H), 4.58 (s, 2H, CH₂O), 3.58-3.56 (m, 6H, CH₂). ¹³C{¹H} NMR (126 MHz, CDCl₃) δ 168.07, 146.87, 122.57, 113.33, 70.38, 69.90, 67.79, 38.85. DART-MS: m/z=339.154 ([M+H]⁺, Calcd; 339.155).

### 3,4,5,6,8,9,12,13,14,15-decahydro-2H,11H-1,7,10,16,4,13-benzotetraoxadiazacyclooctadecine (3)

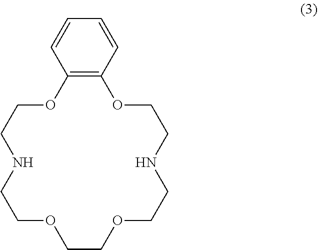 [SMILES: CN1CCOCCOCCNCCOc2ccccc2OCC1]

Under an argon atmosphere, excess borane dimethylsulfide (˜2 mL, 4 eq., 19.56 mmol) was added to a suspension of 2 (1.655 g, 1 eq., 4.89 mmol) in dry THE (50 mL). The reaction was refluxed overnight under argon. After cooling the suspension to room temperature, the reaction was slowly quenched with MeOH (10 mL) during which the solution turned homogenous. Volatiles were removed under vacuum. The white solid obtained was suspended in 6 M HCl (20 mL) and refluxed for 3 h to obtain a homogenous solution. The reaction mixture was cooled to 0° C. and basified (pH>10) by slow addition of an aqueous solution of KOH (10 g, 30 mL). The product was extracted using chloroform (3×30 mL). Organic phase dried over Na₂SO₄. After filtration, volatiles were removed under vacuum to yield a light pink oil which solidified upon standing. The crude product thus obtained was purified by recrystallization from boiling hexane to yield a white solid (1.305 g, 86% yield). ¹H NMR (500 MHz, CDCl₃) δ 6.86 (s, 2H, Ar—CH), 4.11 (t, J=4.7 Hz, 2H, CH₂O), 3.63 (t, J=4.8 Hz, 2H, CH₂O), 3.60 (s, 2H, CH₂O), 3.04 (t, J=4.7 Hz, 2H, CH₂NH), 2.85 (t, J=4.8 Hz, 2H, CH₂NH), 2.23 (s, 1H, NH). ¹³C{¹H} NMR (126 MHz, CDCl₃) δ 148.52, 120.74, 111.98, 70.62, 70.47, 67.94, 49.47, 48.93. DART-MS: m/z=311.196 ([M+H]+, Calcd; 311.196).

### H₂BZmacropa.

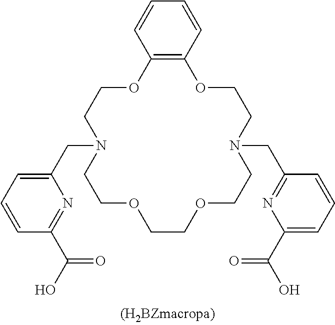

To a solution of methyl 6-(bromomethyl)picolinate (734 mg, 2.1 eq., 3.19 mmol) and 3 (472 mg, 1 eq., 1.52 mmol) in MeCN (50 mL) was added Na₂CO₃ (970 mg, 6 eq., 9.12 mmol) and the reaction mixture was refluxed overnight. After cooling to room temperature, the reaction was filtered, and the filtrate evaporated under vacuum to yield a light-yellow oil. The oil was redissolved in 4 M HCl (15 mL) and the reaction mixture was heated at 90° C. overnight. After cooling to room temperature, the volatiles were removed under vacuum at 60° C. The light-yellow waxy residue thus obtained was completely dissolved in EtOH (10 mL) using sonication and the solution was cooled to 0° C. to induce precipitation. The precipitate was collected via filtration and washed with cold EtOH (2×10 mL). The resulting white solid was redissolved in pure water (3 mL), filtered and filtrate lyophilized to yield H₂BZmacropa·2HCl·3H₂O (690 mg, 64% yield). ¹H NMR (500 MHz, D₂O, pD≈10) δ 7.74 (t, J=7.7 Hz, 2H, Py-CH), 7.66 (d, J=6.7 Hz, 2H, Py-CH), 7.49 (d, J=6.6 Hz, 2H, Py-CH), 6.85 (m, 2H, Ar—CH), 6.79 (m, 2H, Ar—CH), 4.11 (t, J=5.5 Hz, 4H, CH₂O), 3.86 (s, 4H, CH₂Py), 3.73 (t, J=5.3 Hz, 4H, CH₂O), 3.65 (s, 4H, CH₂O), 3.08 (t, J=5.4 Hz, 4H, CH₂N), 2.95 (t, J=5.3 Hz, 4H, CH₂N). ¹³C{¹H} NMR (126 MHz, D₂O, pD≈10) δ 173.49, 158.25, 153.45, 147.46, 138.75, 126.05, 122.77, 121.99, 113.56, 70.10, 68.83, 66.32, 60.15, 54.87, 53.68. ESI-HRMS: m/z=581.259 ([M+H]⁺, Calcd; 581.260), 291.133 ([M+2H]²⁺, Calcd; 291.134). Elemental analysis: found %: C, 51.00, H, 6.15, N 7.94; caled % for C₃₀NH₃₆N₄O₈·2HCl·3H₂O: C 50.92, H 6.27, N 7.92. Analytical HPLC (method quick-MeCN): tR=13.13 min.

Note that in a few batches of ligand synthesis, the purification procedure mentioned above yielded product with <97% purity. In such cases, the product was further purified by preparative HPLC using method prep-MeOH. Analytically pure fractions were combined and volatiles were removed under vacuum. The residue was redissolved thrice in 4 M HCl (10 mL) and evaporated to dryness to remove residual TFA. The resulting white solid was redissolved in pure water (3 mL), filtered and filtrate lyophilized to yield H₂BZmacropa·2HCl·3H₂O (560 mg, 52% yield).

Synthesis and Characterization of H₂BZ₂macropa. H₂BZ₂macropa was prepared according to below procedure.

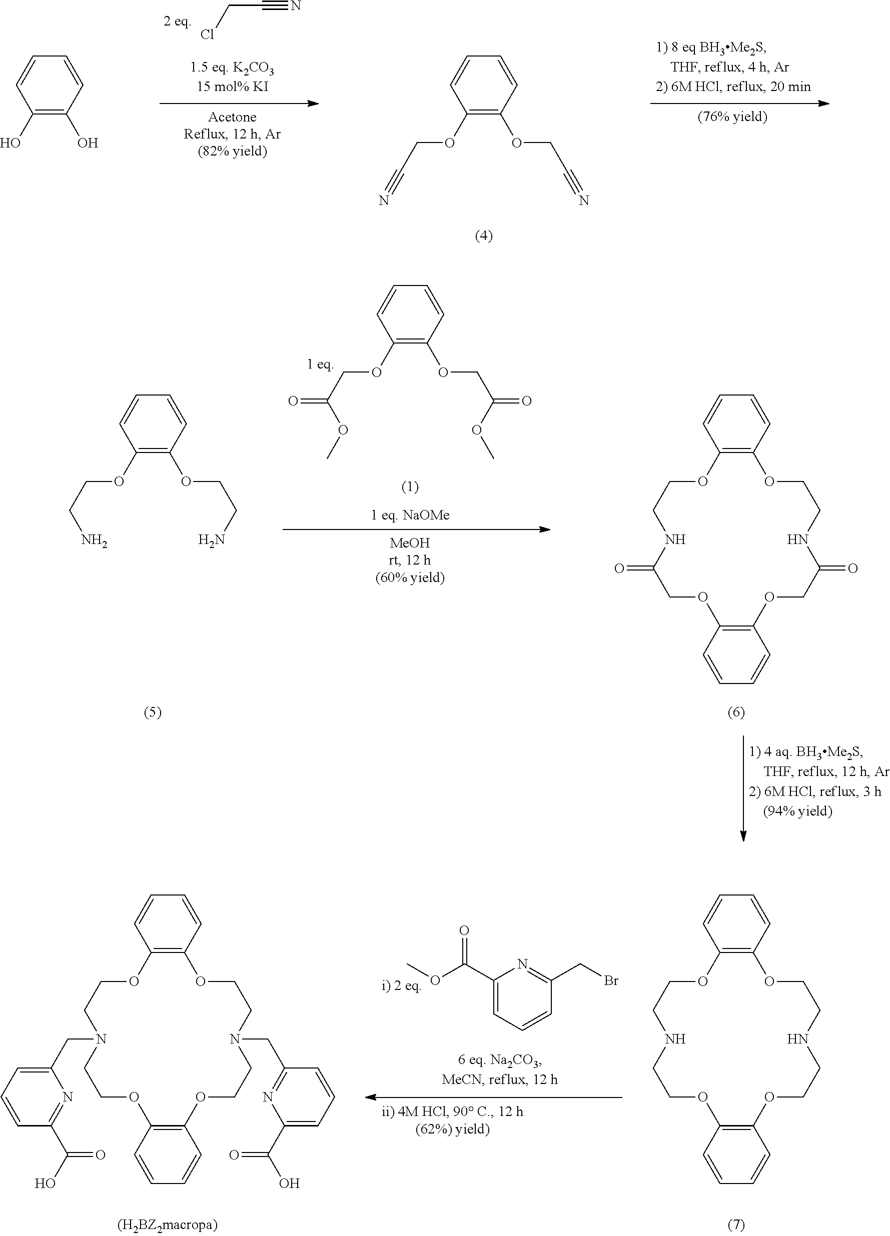

### 1,2-Phenylenedioxydiacetonitrile (4)

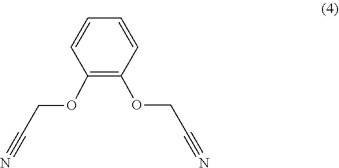 [SMILES: N#CCOc1ccccc1OCC#N]

Chloroacetonitrile (1.7 mL, 3 eq., 27.25 mmol) was added to a solution of catechol (1 g, 1 eq., 9.08 mmol) in acetone (20 mL) followed by K₂CO₃ (1.9 g, 1.5 eq., 13.62 mmol) and KI (226 mg, 0.15 eq., 1.36 mmol). The reaction mixture was refluxed overnight. After cooling to room temperature, the reaction mixture was filtered, and the brown filtrate collected. Volatiles were removed under vacuum to yield a brown residue. The residue was washed with hot diethyl ether (3×30 mL) and the washes collected. Ether was removed under vacuum to obtain a yellow residue. The residue was redissolved in ethanol (20 mL) and cooled in a refrigerator to yield the product as off-white needle crystals (1.4 g, 82% yield). ¹H NMR (500 MHz, CDCl₃) δ 7.12 (m, 4H, Ar—CH), 4.81 (s, 4H, CH₂). ¹³C{¹H} NMR (126 MHz, CDCl₃) δ 147.16, 124.76, 117.05, 115.02, 55.45. DART-MS: m/z=206.092 ([M+NH₄]⁺, Calcd; 206.092).

### 1,2-Bis(2-aminoethoxy)benzene (5)

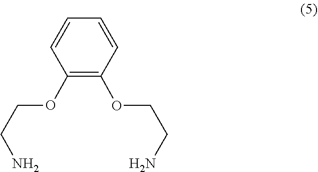 [SMILES: NCCOc1ccccc1OCCN]

Under an argon atmosphere, excess borane dimethylsulfide (˜2 mL, 4 eq., 21.24 mmol) was added to a solution of 4 (1 g, 1 eq., 5.31 mmol) in dry THF (20 mL). The reaction was refluxed overnight under argon. After cooling to room temperature, the reaction was slowly quenched by the dropwise addition of a 1:1 mixture of water and THE (20 mL). Volatiles were removed under vacuum and the white solid obtained was suspended in 6 M HCl (20 mL). The reaction was refluxed for 1 h to obtain a homogenous solution. The reaction mixture was cooled to 0° C. and basified (pH>10) by slow addition of an aqueous solution of KOH (10 g, 30 mL). The product was extracted using chloroform (3×30 mL) and the collected organic phase was washed with saturated brine (2×25 mL). Organic phase was dried over Na₂SO₄. After filtration, volatiles were removed under vacuum to yield a light-yellow oil, which was stored under Argon/vacuum (0.790 g, 76% yield). ¹H NMR (500 MHz, CDCl₃) δ 6.92 (s, 2H, Ar—CH), 4.03 (t, J=5.2 Hz, 2H, CH₂O), 3.09 (t, J=5.2 Hz, 2H, CH₂N). ¹³C{¹H} NMR (101 MHz, CDCl₃) δ 149.23, 121.78, 114.84, 71.92, 41.81. DART-MS: m/z=197.128 ([M+H]⁺, Calcd; 197.128).

### 9,10,18,19-Tetrahydro-6H,17H-dibenzo[b,k][1,4,10,13,7,16]-tetraoxadiazacyclooctadecine-7,20(8H,21H)-dione (6)

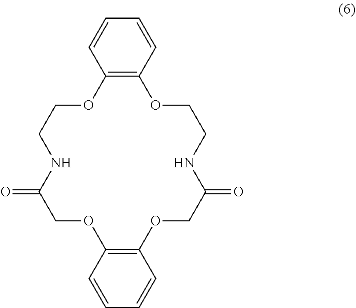 [SMILES: O=C1COc2ccccc2OCC(=O)NCCOc2ccccc2OCCN1]

A homogenous solution of 1 (0.920 g, 1 eq., 3.62 mmol), 5 (0.710 g)²⁹¹ g, 1 eq., 3.62 mmol), and sodium methoxide (0.196 g, 1 eq., 3.62 mmol) in MeOH (40 mL) was stirred at room temperature overnight. A lot of precipitate formed during the course of the reaction. The reaction mixture was cooled in a refrigerator for 3 h and the precipitate was collected via filtration while cold. The collected precipitate was washed with cold MeOH (20 mL) and dried under vacuum to yield the product as a white solid (0.840 g, 60% yield). ¹H NMR (500 MHz, CDCl₃) δ 7.23 (s, 1H, NH), 6.98 (m, 1H, Ar—CH), 6.92 (m, 2H, Ar—CH), 6.84 (m, 1H, Ar—CH), 4.57 (s, 2H, CH₂O), 4.10 (m, 2H, CH₂O), 3.88 (q, J=5.2 Hz, 2H, CH₂NH). ¹³C{¹H} NMR (126 MHz, CDCl₃) δ 167.98, 147.85, 146.51, 122.56, 121.45, 113.26, 111.98, 67.63, 66.96, 38.64. DART-MS: m/z=387.154 ([M+H]⁺, Calcd; 387.155).

### 7,8,9,10,18,19,20,21-Octahydro-6H,17H-dibenzo[b,k][1,4,10,13,7,16]tetraoxadiazacyclooctadecine (7)

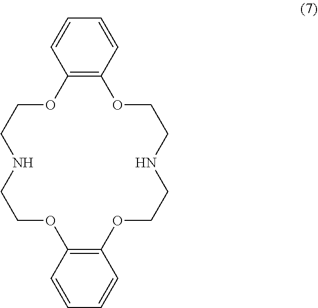 [SMILES: c1ccc2c(c1)OCCNCCOc1ccccc1OCCNCCO2]

Under an argon atmosphere, excess borane dimethylsulfide (˜1.5 mL, 4 eq., 15.53 mmol) was added to a suspension of 6 (1.5 g, 1 eq., 3.88 mmol) in dry THE (50 mL). The reaction mixture was refluxed overnight under argon. After cooling the suspension to room temperature, the reaction was slowly quenched with MeOH (10 mL) during which the solution turned homogenous. Volatiles were removed under vacuum. The white solid obtained was suspended in 6 M HCl (20 mL) and refluxed for 3 h to obtain a homogenous solution. The reaction mixture was cooled to 0° C. and basified (pH>10) by slow addition of an aqueous solution of KOH (10 g, 30 mL). The product was extracted using chloroform (3×30 mL). Organic phase dried over Na₂SO₄. After filtration, volatiles were removed under vacuum to yield the product as a white solid (1.310 g, 94% yield). ¹H NMR (500 MHz, CDCl₃) δ 6.86 (m, 4H, Ar—CH), 4.12 (t, J=4.7 Hz, 4H, CH₂O), 3.14 (t, J=4.7 Hz, 4H, CH₂NH), 2.40 (s, 1H, NH). ¹³C{¹H} NMR (126 MHz, CDCl₃) δ 148.26, 120.67, 111.28, 67.64, 49.16. DART-MS: m/z=359.195 ([M+H]⁺, Calcd; 359.196).

### H₂BZ₂macropa

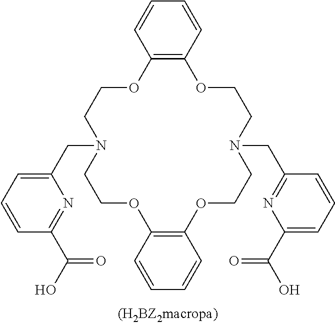 [SMILES: O=C(O)c1cccc(CN2CCOc3ccccc3OCCN(Cc3cccc(C(=O)O)n3)CCOc3ccccc3OCC2)n1]

To a solution of methyl 6-(bromomethyl)picolinate (580 mg, 2.1 eq., 2.52 mmol) and 7 (430 mg, 1 eq., 1.2 mmol) in MeCN (50 mL) was added Na₂CO₃ (763 mg, 6 eq., 7.2 mmol) and the reaction mixture was refluxed overnight. After cooling to room temperature, the reaction was filtered, and the filtrate evaporated under vacuum to yield a light-yellow oil. The oil was redissolved in 4 M HCl (15 mL) and the reaction mixture was heated at 90° C. overnight. A lot of precipitate formed over the course of the reaction. After cooling to room temperature, the precipitate was collected via filtration and washed with ice cold water (2×20 mL) followed by acetone (2×20 mL). The solid residue was resuspended in pure water (3 mL) and lyophilized to yield H₂BZ₂macropa·2HCl·2.5H₂O (555 mg, 62% yield). ¹H NMR (500 MHz, D₂O, pD≈10) δ 7.58 (t, J=7.7 Hz, 1H, Py-CH), 7.51 (d, J=7.8 Hz, 1H, Py-CH), 7.39 (d, J=7.8 Hz, 1H, Py-CH), 6.81 (m, 4H, Ar—CH), 4.11 (t, J=5.3 Hz, 4H, CH₂O), 3.81 (s, 2H, CH₂Py), 3.14 (t, J=5.4 Hz, 4H, CH₂N). ¹³C{¹H} NMR (126 MHz, D₂O, pD≈10) δ 173.06, 158.37, 153.11, 147.32, 138.55, 125.57, 122.67, 121.91, 113.02, 65.70, 59.59, 54.64. ESI-HRMS: m/z=629.261 ([M+H]⁺, Calcd; 629.260). Elemental analysis: found %: C, 54.72, H, 5.90, N, 7.52; calcd % for C₃₄H₃₆N₄O₈·2HCl·2.5H₂O: C 54.70, H 5.81, N 7.50. Analytical HPLC (method quick-MeCN): tR=13.81 min.

Synthesis and Characterization of H₂BZmacropa-NCS. H₂BZ₂macropa was prepared according to below procedure.

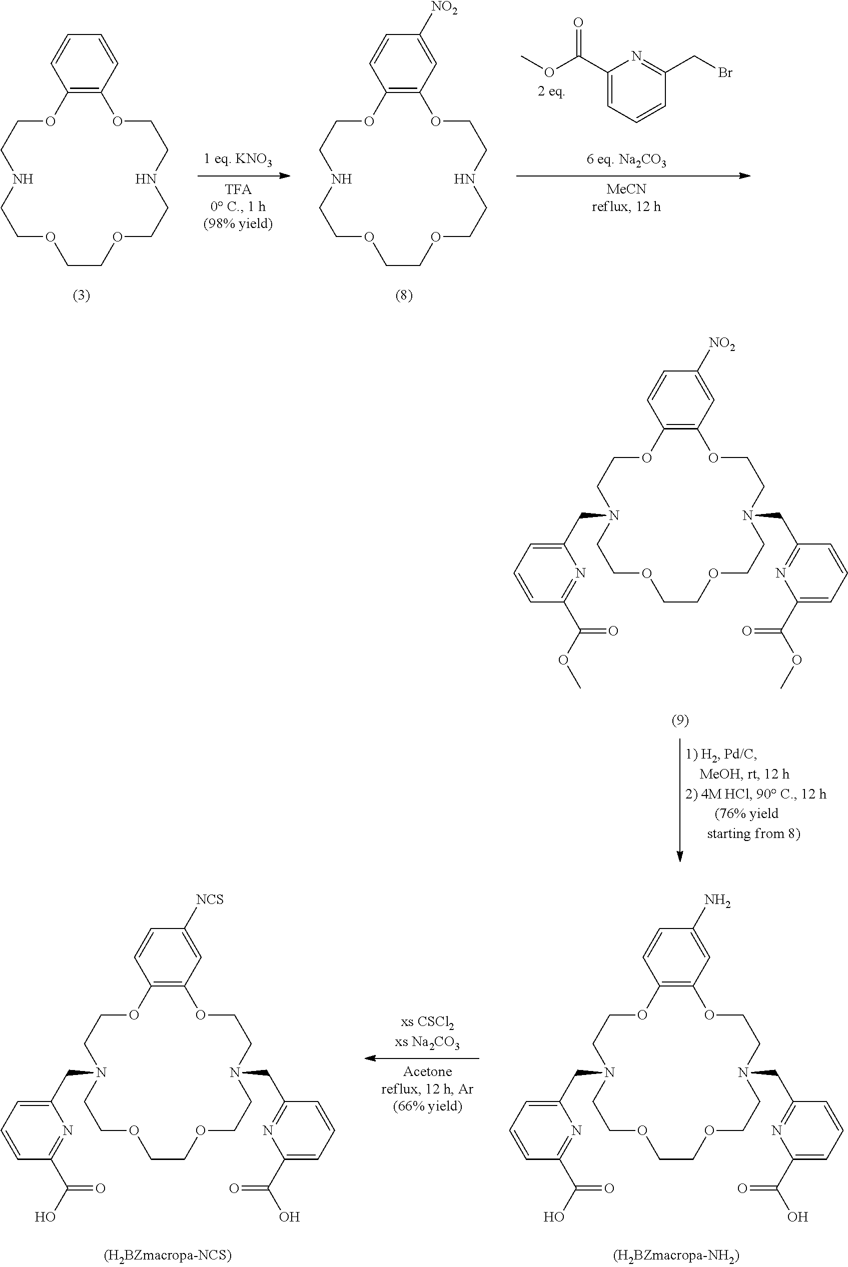

### 18-Nitro-3,4,5,6,8,9,12,13,14,15-decahydro-2H,11H-1,7,10,16,4,13-benzotetraoxadiazacyclooctadecine (8)

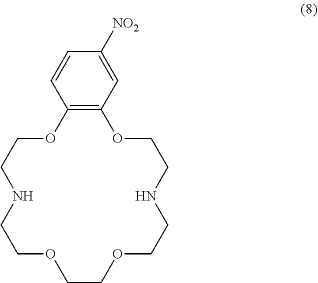 [SMILES: O=[N+]([O-])c1ccc2c(c1)OCCNCCOCCOCCNCCO2]

In a 50 mL round bottom flask, trifluoroacetic acid (20 mL) was cooled to 0° C. Compound 3 (1 g, 1.0 eq., 3.22 mmol) was added to the cold trifluoroacetic acid in small batches over 1 h to yield a light teal colored homogenous solution. An ice-cold solution of KNO₃ (326 mg, 1.0 eq., 3.22 mmol) in trifluoroacetic acid (10 mL) was slowly (˜10 mins) added to the reaction mixture at 0° C., during which the reaction color turned yellow. The reaction was allowed to stir at 0° C. for another 1 h. Trifluoroacetic acid was removed under vacuum. The yellow oil thus obtained was basified using slow addition of saturated aqueous NaHCO₃(50 mL) and the product was extracted using DCM (3×30 mL). Organic phase dried over Na₂SO₄. After filtration, volatiles were removed under vacuum to yield the product as a yellow solid (1.120 g, 98% yield). ¹H NMR (500 MHz, CDCl₃) δ 7.87 (dd, J=9.0, 2.6 Hz, 1H, Ar—CH), 7.71 (d, J=2.6 Hz, 1H, Ar—CH), 6.87 (d, J=9.0 Hz, 1H, Ar—CH), 4.19 (m, 4H, CH₂O), 3.63 (m, 4H, CH₂O), 3.60 (s, 4H, CH₂O), 3.07 (t, J=4.6 Hz, 4H, CH₂NH), 2.86 (t, J=4.6 Hz, 4H, CH₂NH), 2.25 (s, 2H, NH). ¹³C{¹H} NMR (126 MHz, CDCl₃) δ 154.13, 148.34, 141.35, 117.73, 110.33, 106.88, 70.48, 70.44, 70.39, 69.14, 68.77, 49.47, 49.43, 48.46, 48.43. ESI-HRMS: m/z=356.180 ([M+H]⁺, Calcd; 356.181).

### Dimethyl-6,6′-((18-nitro-2,3,5,6,8,9,11,12,14,15-decahydro-4H,13H-benzo[b][1,4,10,13]tetraoxa[7,16]diazacyclooctadecine-4,13-diyl)bis(methylene))dipicolinate (9)

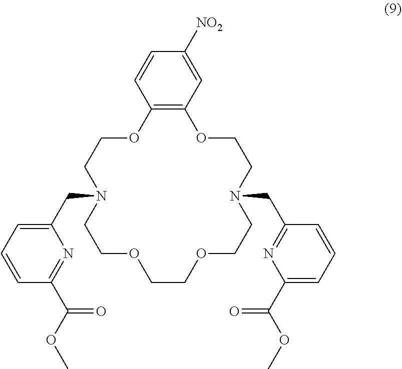

To a solution of methyl 6-(bromomethyl)picolinate (647 mg, 2.0 eq., 2.81 mmol) and 8 (500 mg, 1 eq., 1.41 mmol) in MeCN (50 mL) was added Na₂CO₃ (897 mg, 6 eq., 8.46 mmol) and the reaction mixture was refluxed overnight. After cooling to room temperature, the reaction was filtered, and the filtrate evaporated under vacuum to yield a yellow oil which solidified upon standing. The product thus obtained was used for the nest step without further purification/characterization. ESI-HRMS: m/z=654.275 ([M+H]⁺, Calcd; 654.277).

### H₂BZmacropa-NH₂

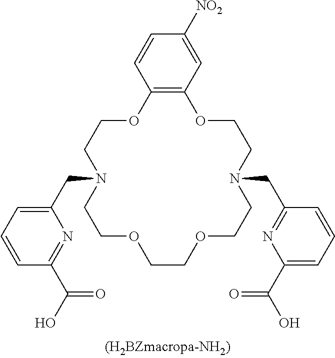

The oil (9) obtained from the above reaction (assuming full conversion; 1 eq., 1.41 mmol) was dissolved in MeOH (10 mL) in a 50 mL round bottom flask and 10% Pd/C (3 mol % loading, 45 mg, 0.04 mmol Pd) added. The round bottom flask was purged with a balloon of H₂ and the reaction was stirred under H₂ at room temperature overnight. The gray mixture was filtered through a bed of Celite and the bed was washed with MeOH (20 mL×4). The combined filtrate and washes were dried under vacuum to yield a light brown oil to which 4 M HCl (20 mL) was added, and the reaction was heated at 90° C. overnight. After cooling to room temperature, the volatiles were removed under vacuum at 60° C. The light-brown wax thus obtained was redissolved in 6 mL MeOH:H₂O (5:95) mixture containing 0.1% TFA and purified by preparative HPLC using method prep-MeOH_slow. Analytically pure fractions were combined, and volatiles were removed under vacuum. The residue was redissolved thrice in 4 M HCl (10 mL) and evaporated to dryness to remove residual TFA. The resulting light-yellow solid was redissolved in pure water (3 mL), filtered and filtrate lyophilized to yield H₂BZmacropa-NH₂·3HCl (754 mg, 76% combined yield starting from 8). ¹H NMR (500 MHz, D₂O, pD≈5) δ 7.78 (m, 2H, Py-CH), 7.72 (d, J=7.8 Hz, 1H, Py-CH), 7.69 (d, J=7.7 Hz, 1H, Py-CH), 7.53 (d, J=7.6 Hz, 2H, Py-CH), 6.49 (d, J=8.5 Hz, 1H, Ar—CH), 6.26 (dd, J=8.5, 2.4 Hz, 1H, Ar—CH), 6.17 (d, J=2.4 Hz, 1H, Ar—CH), 4.55 (s, 2H, CH₂Py), 4.53 (s, 2H, CH₂Py), 4.17 (q, J=5.0 Hz, 4H), 3.97 (t, J=4.9 Hz, 4H), 3.75 (s, 4H, CH₂O), 3.69 (t, J=4.7 Hz, 4H), 3.59 (t, J=4.8 Hz, 2H), 3.56 (t, J=4.7 Hz, 2H). ¹³C{¹H} NMR (126 MHz, D₂O, pD≈5) δ 172.31, 153.12, 152.98, 147.05, 140.45, 139.60, 139.54, 126.20, 126.10, 124.13, 124.03, 114.22, 109.67, 103.09, 70.50, 70.47, 65.58, 65.45, 63.61, 63.24, 59.16, 59.09, 56.57, 56.37, 54.61. ESI-HRMS: m/z=596.271 ([M+H]⁺, Calcd; 596.271), 298.639 ([M+2H]²⁺, Calcd; 298.639).

### H₂BZmacropa-NCS

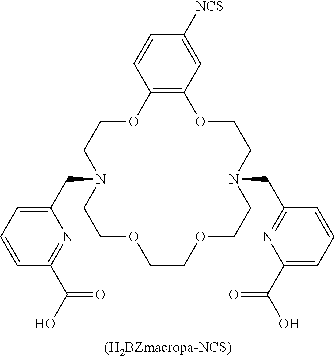

A suspension of H₂BZmacropa-NH₂·3HCl (113 mg, 1 eq., 0.16 mmol) and Na₂CO₃ (254 mg, 15 eq., 2.4 mmol) in acetone (10 mL) was heated at reflux under an argon atmosphere for 30 min before the slow addition of CSCl₂ (762 μL, 50 eq., 8 mmol, 80-85%, Acros Organics). The orange reaction mixture was refluxed overnight under argon. After cooling to room temperature, volatiles were removed under vacuum and the residue was dissolved in 4 mL MeCN:H₂O(10:90) mixture containing 0.1% TFA, filtered, and immediately purified by preparative HPLC using method prep-MeCN. Analytically pure fractions were combined, and volatiles were removed under vacuum. The resulting light-beige solid was redissolved in pure water (2 mL), filtered and filtrate lyophilized to yield H₂BZmacropa-NCS·2TFA (92 mg, 66% yield). No. of TFA calculated from ¹H NMR and ¹⁹F NMR spectra of the sample spiked with fluorobenzene. ¹H NMR (500 MHz, D₂O, pD≈4) δ 7.78 (m, 2H, Py-CH), 7.68 (t, J=7.8 Hz, 2H, Py-CH), 7.53 (t, J=7.6 Hz, 2H, Py-CH), 6.73 (dd, J=8.7, 2.3 Hz, 1H, Ar—CH), 6.54 (s, 1H, Ar—CH), 6.53 (d, J=2.2 Hz, 1H, Ar—CH), 4.71 (s, 4H, CH₂Py), 4.26 (t, J=4.5 Hz, 2H), 4.22 (t, J=4.6 Hz, 2H), 4.05 (t, J=4.9 Hz, 4H), 3.90-3.84 (m, 4H), 3.82 (s, 4H, CH₂O), 3.77-3.70 (m, 4H). ¹³C{¹H} NMR (126 MHz, D₂O, pD≈4) δ 171.72, 171.52, 152.79, 152.74, 150.11, 150.10, 146.31, 145.62, 139.91, 139.87, 133.79, 125.98, 125.96, 124.36, 124.25, 119.40, 112.69, 110.26, 70.71, 64.79, 62.73, 59.41, 59.30, 57.20, 57.02, 54.53, 54.46. ESI-HRMS: m/z=638.228 ([M+H]⁺, Calcd; 638.227). Analytical IPLC (method quick-MeCN): tR=14.92 min.

H₂BZmacropa-NCS Hydrolysis. The stability of BZmacropa-NCS²— towards hydrolysis was tested as the followings. A solution (1 mL) of H₂BZmacropa-NCS (≈1.5 mM) was prepared in 0.1 M Na₂CO₃/NaHCO₃pH 9.1 buffer and stored at room temperature (≈23±1° C.) in a vial covered with aluminum foil. This solution was analyzed periodically by analytical HPLC (method quick-MeCN, 5 μL aliquots) for over a week. It shows that H₂macropa-NCS is relatively stable towards hydrolysis of the thiocyanate functional group with a half-life of 56 h (more than a week's time for complete hydrolysis).

### Example 2: Coordination Chemistry with La³⁺ and/or Ba²⁺

Thermodynamic stability studies. Thermodynamic stability studies were carried out Using Ba(II) and La(III) as cold surrogates for ²²³Ra and ²²⁵Ac. The protonation constants of BZmacropa and the thermodynamic metal stability constants obtained by potentiometric titrations are summarized below in Table 1 (I=0.1 M KCl, 25° C.). The conditional thermodynamic stability constants for barium complexes and lanthanum complexes are summarized in Table 2.

TABLE 1

| | BZmacropa | | macropa | | DOTA | | DTPA | EDTA |
|---|---|---|---|---|---|---|---|---|
| log K₁ | 7.06 | (2) | 7.41 | (1) | 11.14 | (1) | 10.34 | 10.19 |
| log K₂ | 6.41 | (0) | 6.90 | (3) | 9.69 | (2) | 8.59 | 6.13 |
| log K₃ | 3.33 | (3) | 3.23 | (1) | 4.85 | (2) | 4.25 | 2.69 |
| log K₄ | 2.49 | (3) | 2.45 | (5) | 3.95 | (1) | 2.71 | 2.00 |
| log K₅ | | | | | | | 2.18 | |

| log K_{BaL} | 9.09 | 11.11 | (4) | 11.75 | (1) | 8.78 | 7.80 |
|---|---|---|---|---|---|---|---|
| log K_{BaHL} | 3.56 | 3.76 | (2) | | | 5.34 | |
| log K_{BaH2L} | — | 2.49 | (7) | | | | |

| log K_{LaL} | 13.98 | 14.99 | (2) | 21.7 | 19.48 | 15.46 |
|---|---|---|---|---|---|---|
| log K_{LaHL} | — | 2.28 | (3) | — | — | — |

TABLE 2

| | BZmacropa | macropa | DOTA | DTPA | EDTA |
|---|---|---|---|---|---|
| log K_{Ba}′ | 8.91 | 10.74 | 5.72 | 4.63 | 4.99 |
| log K_{La}′ | 13.80 | 14.63 | 15.67 | 15.32 | 12.65 |
| pBa | 9.87 | 11.69 | 6.76 | 6.15 | 6.28 |
| pLa | 14.76 | 15.58 | 16.62 | 16.28 | 13.60 |

*Conditional stability constants (log K′_{M}) at pH 7.4, 25° C., and I = 0.1M KCl
**pM values calculated from −log [M]_{free} ([M]ₜₒₜ = 10⁻⁶ M, [L]ₜₒₜ = 10⁻⁵ M, pH 7.4, 25° C., and I = 0.1M KCl)

X-Ray Diffraction Studies. Treatment of equimolar ratios of LaCl₃ with each ligand (H₂BZmacropa or H₂BZ₂macropa) in water at neutral pH followed by salt metathesis with KPF₆ led to the precipitation and thus isolation of [LaL(H₂O)]PF₆ complexes ([La(BZmacropa)(H₂O)](PF₆) and [La(BZ₂macropa)(H₂O)](PF₆)). Crystals suitable for X-ray crystallography were obtained by slow evaporation of a concentrated aqueous solution of [LaL(H₂O)]PF₆ complexes ([La(BZmacropa)(H₂O)](PF₆) and [La(BZ₂macropa)(H₂O)](PF₆)) at room temperature over a few days.

Low-temperature X-ray diffraction data for the La(III) complexes were collected on a Rigaku XtaLAB Synergy diffractometer coupled to a Rigaku Hypix detector with Mo Kα radiation (λ=0.71073 Å), from a PhotonJet micro-focus X-ray source at 100 K. The diffraction images were processed and scaled using the CrysAlisPro software. The structures were solved through intrinsic phasing using SHELXT¹² and refined against F² on all data by full-matrix least squares with SHELXL following established refinement strategies. All non-hydrogen atoms were refined anisotropically. All hydrogen atoms bound to carbon were included in the model at geometrically calculated positions and refined using a riding model. Hydrogen atoms bound to oxygen were located in the difference Fourier synthesis and subsequently refined semi-freely with the help of distance restraints. The isotropic displacement parameters of all hydrogen atoms were fixed to 1.2 times the Ueq value of the atoms they are linked to (1.5 times for methyl groups). It shows all the O and N donor atoms on the ligand binds to La³⁺ forming a decadentate coordination sphere and a 11^{th} coordination site on an axial position is filled by a water molecule.

Potentiometric Titrations. Stability constant measurements were carried out by titrating an aqueous solution (˜15 mL) of ligand (˜1 mM), metal (˜1 mM), and HCl (˜10 mM) with standardized KOH (0.1 M). The ionic strength of the solution was maintained at 0.1 M using KCl. The titration method employed a 0.1 mV/min drift limit with a minimum and maximum wait time of 0 s and 300 s respectively between addition of KOH aliquots (0.015 mL volume increments). No metal hydroxide precipitation was observed within the pH range and concentration employed. The titration data within the pH range of 2.2-11.3 were analyzed using Hyperquad2013 software. The stability constants were calculated from the average of three independent titrations (using three independently prepared ligand stock solutions from three independent synthetic preparations of ligands). Stability constants (log K_{LaL}) of lanthanum-ligand complexes determined by potentiometric titrations of the ligands in the presence of equimolar La³⁺ ions revealed a decreased stability upon addition of phenyl groups to the backbone; log K_{LaL} H₂macropa>H₂BZmacropa>H₂BZ₂macropa. However, this ranking does not detract from the fact that the data showed H₂BZmacropa and H₂BZ₂macropa form highly stable complexes with La³⁺.

### Example 3: Actinium-225 Chelation and Biodistribution Studies

Radiolabeling of Ligands with Actinium-225. A solution of gentisic acid (10 mg/mL) in water (10 μL) was added to a solution of ²²⁵Ac(NO₃)₃ in 0.1 M HNO₃ (70 μCi in 25 μL). To this mixture was added 2.5 μL NH₄OAc solution (5 M, pH 7), followed by 5 μL of a solution of chelator in water (2 mg/mL). The pH of the resulting solution was 5.5. This mixture was incubated at RT for 30 min, then a 1 μL aliquot was analyzed via iTLC (condition 1). Both ligands (H₂BZmacropa and H₂BZ₂macropa) quantitatively coordinated 225Ac³⁺ in 30 minutes.

### Antibody Conjugation.

The following conjugates were prepared:
GC33-BZM: GC33-BZmacropa conjugate.
GC33-M: GC33-macropa conjugate.
OBI-BZM: Obinutuzumab-BZmacropa conjugate.
   Concentrated antibody stock solutions were thawed and buffered exchanged into PBS using a PD-10 column. The resulting solutions were then mixed with solutions of H₂macropa-NCS (3 equiv.) or H₂BZmacropa-NCS (2.5 equiv.) in pure water and PBS was added to a final volume of 900 μL. A solution of 0.1 M bicarbonate buffer (100 μL, pH 9.5) was added to adjust the pH to 9.2, and the mixture was heated at 37° C. with shaking for 1 h. The solution was then purified using a PD-10 column pre-equilibrated with 0.1 M NH₄OAc, pH 7. Aliquots of the product were immediately frozen at −30° C. and stored for no more than 1 month prior to use.

Chelator:Antibody Ratios: The chelate:antibody ratio was investigated using arsenazo assay. A mixture of arsenazo(III) dye (ARS) and La(NO₃)₃ (400 μM ARS, 200 μM La) was prepared in 0.1 M NH₄OAc, pH 7. This solution was mixed 1:1 with known concentrations of chelator or antibody conjugate in 0.1 M NH₄OAc. The mixtures were incubated at least 30 min prior to spectroscopic analysis. The absorbance of the solutions at 660 nm was divided by the absorbance at 570 nm (isosbestic point). This ratio was plotted vs chelate concentration for the known samples and fit using a linear regression to yield a standard curve. Chelate concentration in unknown samples was calculated by interpolation using the standard curve (EDTA or unconjugated H₂BZmacropa). This concentration was then divided by the protein concentration in the samples to yield the chelate:antibody ratio. Protein concentration in the samples was determined using the Pierce BCA Assay Kit (Thermo Fisher, Waltham, MA) according to the manufacturer's instructions. A solution of trastuzumab was used for the standard curve. All conjugates were found to have chelate:antibody ratios of approximately 1:1 using this method.

Binding Affinity of GC33-BZM, GC33-M, and Unmodified GC33. The binding affinity of GC33-BZM, GC33-M, and unmodified GC33 were investigated using bio-layer interferometry (BLI). Biotinylated human GPC3 protein (Acro Biosystems, Newark, DE) was diluted to 1 μg/mL in assay buffer: 1×PBS with 0.02% Tween-20 and 0.1% acetylated BSA (Electron Microscopy Sciences, Hatfield, PA) in a 96-well plate and loaded onto streptavidin biosensors (ForteBio, Menlo Park, CA). Antibodies were diluted in assay buffer at 4 concentrations from 0-25 nM and loaded into a 96-well plate (final volume of 200 μL). GPC3 was loaded to a density of 0.3 to avoid avidity affects. Reference wells containing buffer only, GPC3-only and antibody only were used as blanks. Plates were run on an Octet Red96 system (FortéBio, Menlo Park, CA) and analyzed with ForteBio Octet Data Analysis software (v.11). The exposure of various concentrations of antibody to immobilized GPC3 antigen revealed concentration-dependent binding of the antibody to the surface and subsequent dissociation. The combined association and dissociation kinetic data across all concentrations were fit using the global fitting model in the Octet Analysis Studio software. Data revealed K_{d} values of 0.19, 0.14, and 0.042 nM for GC33-BZM, GC33-M, and GC33 respectively. Taken together, the results show that both GC33-BZM and GC33-M retained high binding affinity for GPC3.

Radiolabeling of Antibody Conjugates GC33-BZM, GC33-M, OBI-BZM. A solution of gentisic acid (10 mg/mL) in water (10 μL) was added to a solution of ²²⁵Ac(NO₃)₃ in 0.1 M HNO₃ (150 μCi, 5.55 MBq in 20 μL). To this mixture was added 2 μL NH₄OAc solution (5 M, pH 7), followed by a solution of antibody conjugate (75 μL, 450 μg, 3 nmol) in 0.1 M NH₄OAc (pH 7). The mixture (final pH 5.5) was incubated at room temperature for 30 min, then a 1 μL aliquot was analyzed via ITLC (condition 2). After confirming radiolabeling, the mixture was purified using a PD-10 column pre-equilibrated with PBS. The final product was allowed to equilibrate for at least 2 h following purification before use in other studies.

Radioconjugate Stability Experiments in Human Serum. Solutions of ²²⁵Ac conjugates in PBS were diluted with whole human serum to a final concentration of 10 μCi (0.37 MBq) conjugate in 500 μL of at least 90% serum. The mixtures were spotted initially (t=0), then incubated with shaking at 37° C. and monitored via ITLC. Unconjugated complexes were monitored using ITLC condition 1, whereas antibody conjugates were analyzed using ITLC condition 2. ITLC plates were allowed to equilibrate for at least 24 h prior to analysis. Activity at the origin (0-10 mm) was considered intact complex, while mobile activity (>10 mm) was considered unchelated ²²⁵Ac. Stability experiments with unconjugated chelators were performed on single samples monitored over 5 days. Studies of antibody conjugates were performed in triplicate and monitored daily over 7 days. The radioconjugate ²²⁵Ac[Ac]-GC33-BZM exhibited degradation over the course of the experiment, with approximately 55% intact complex remaining after 7 days.

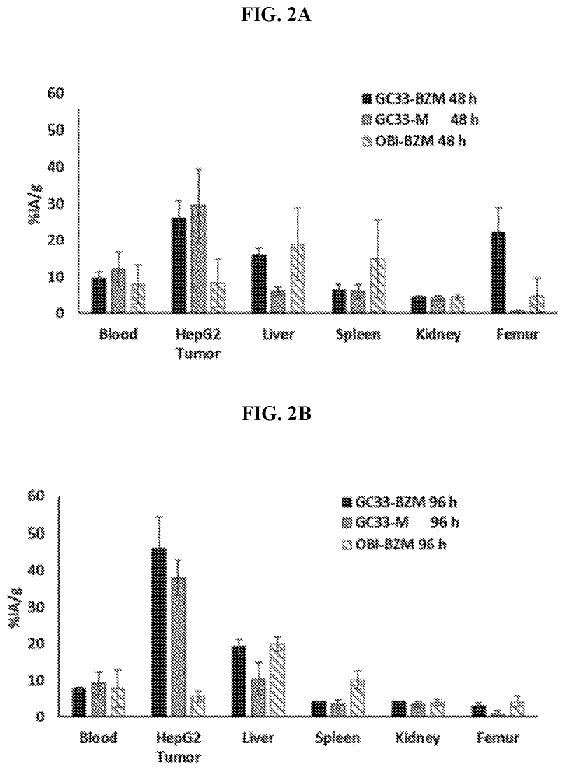

Biodistribution. Mice were injected intravenously with 100 nCi (3.7 kBq, 300 ng) of GC33-BZM, GC33-M, or OBI-BZM (isotype control) dissolved in PBS containing 10 mg/mL BSA (100 μL). At 48 and 96 h post-injection, mice (n=3-4 per time point) were euthanized. Their organs were harvested and weighed, then counted on a gamma counter after allowing 24 h for ²²⁵Ac daughters to equilibrate. The activity in each organ was decay-corrected using a standard of known activity prepared at the same time as the injections. Radiotracer uptake in each organ was calculated by dividing the % injected activity (% IA) in each organ by the organ's weight. Measurement errors are reported as the standard deviation of the % IA/g values (FIGS. 2A-2B). In this study, ²²⁵Ac[Ac]-GC33-BZM demonstrated clear, specific tumor uptake at both 48 and 96 h post-injection, with tumor signal greater than that of all organs at both time points. Tumor targeting by ²²⁵Ac[Ac]-GC33-BZM also showed no significant difference from that of ²²⁵Ac[Ac]-GC33-M, indicating comparable tumor targeting (FIGS. 2A-2B). While the off-target accumulation of ²²⁵Ac[Ac]-GC33-BZM was higher than that of ²²⁵Ac[Ac]-GC33-M in both the liver and femur at 48 h for these particular conjugates (FIG. 2A), the data clearly evidences the suitability of ²²⁵Ac[Ac]-GC33-BZM and other compositions of the present technology in targeted radiotherapy.

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A compound of Formula (I)

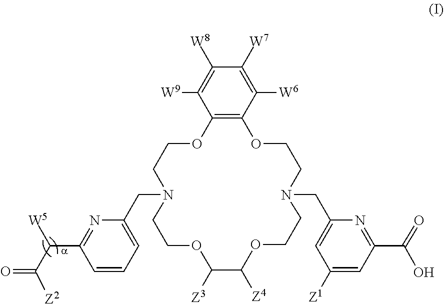

or a pharmaceutically acceptable salt and/or solvate thereof, wherein
Z¹ is H or —X¹—W¹;
Z² is OH or NH—W²;
Z³ is H or W³, and Z⁴ is H or W⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W¹, W², W⁶, W⁷, W⁸, and W⁹ are each independently H, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)_{y}—R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group;
W³, W⁴, W⁵, and W¹⁰ are each independently OH, NH₂, SH, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)yₓ-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group; and
R′ is independently at each occurrence H, halo, —N₃, C₁-C₆ alkyl, C₃-C₆ cycloalkyl, C₂-C₆ alkenyl, C₅-C₅ cycloalkenyl, C₂-C₆ alkynyl, C₅-C₁₀ cycloalkynyl, C₅-C₆ aryl, heterocyclyl, or heteroaryl.
B. The compound of Paragraph A, wherein at least one of W⁶, W⁷, W⁸, and W⁹ is not hydrogen.
C. The compound of Paragraph A or B, wherein the compound is a compound of Formula (I-A)

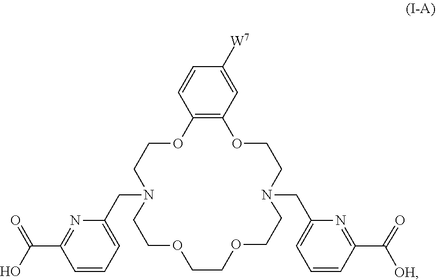

or a pharmaceutically acceptable salt and/or solvate thereof.
D. The compound of any one of Paragraphs A-C, wherein the compound is

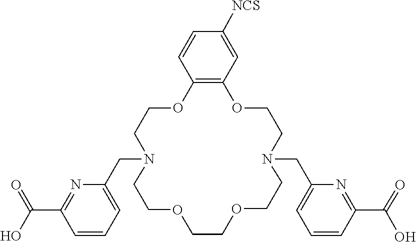

or pharmaceutically acceptable salt and/or solvate thereof.
E. The compound of Paragraph A, wherein the compound of Formula (I) is a compound of Formula (I-B)

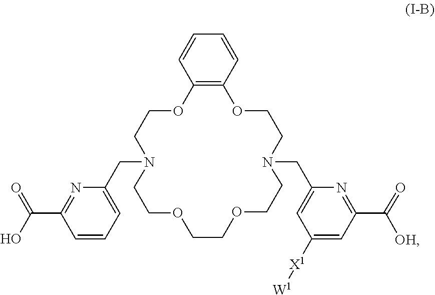

or a pharmaceutically acceptable salt and/or solvate thereof.
F. The compound of Paragraph A, wherein the compound of Formula (I) is a compound of Formula (I-C)

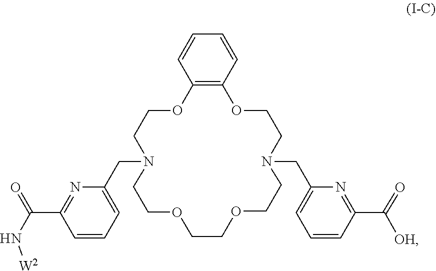

or a pharmaceutically acceptable salt and/or solvate thereof.
G. The compound of Paragraph A, wherein the compound of Formula (I) is a compound of Formula (I-D)

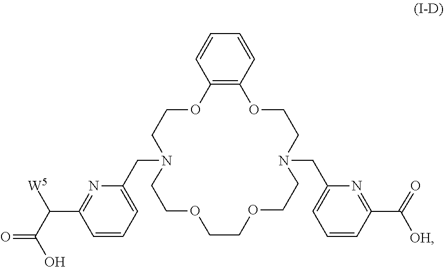

or a pharmaceutically acceptable salt and/or solvate thereof.
H. The compound of Paragraph A, wherein the compound is a compound of Formula (I-E)

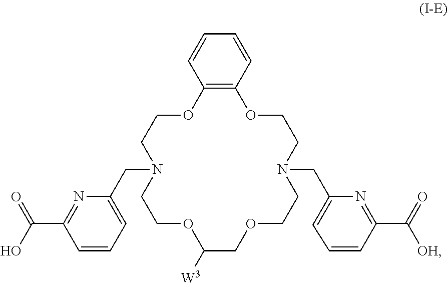

or a pharmaceutically acceptable salt and/or solvate thereof.
I. The compound of Paragraph A, wherein the compound is a compound of Formula (I-F)

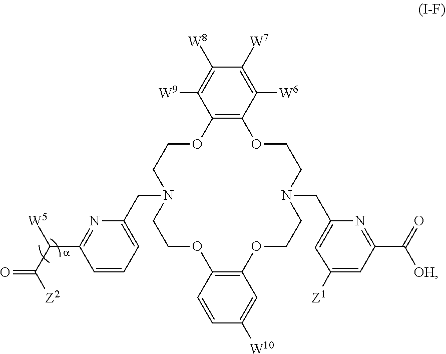

or a pharmaceutically acceptable salt and/or solvate thereof.
J. A compound of Formula (II)

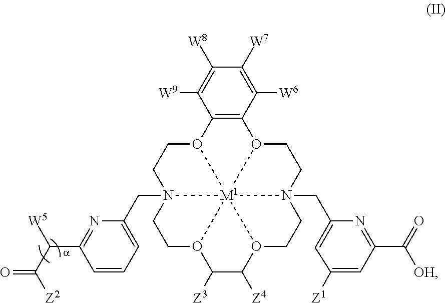

or a pharmaceutically acceptable salt and/or solvate thereof, wherein
M¹ is a radionuclide;
Z¹ is H or —X¹—W¹;
Z² is OH or NH—W²;
Z³ is H or W³, and Z⁴ is H or W⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W¹, W², W⁶, W⁷, W⁸, and W⁹ are each independently H, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)_{y}—R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group;
W³, W⁴, W⁵, and W¹⁰ are each independently OH, NH₂, SH, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)yₓ-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group; and
R′ is independently at each occurrence H, halo, —N₃, C₁-C₆ alkyl, C₃-C₆ cycloalkyl, C₂-C₆ alkenyl, C₅-C₅ cycloalkenyl, C₂-C₆ alkynyl, C₅-C₁₀ cycloalkynyl, C₅-C₆ aryl, heterocyclyl, or heteroaryl.
K. The compound of Paragraph J, wherein at least one of W⁶, W⁷, W⁸, and W⁹ is not hydrogen.
L. The compound of Paragraph J or K, wherein the compound of Formula (II) is a compound of Formula (II-A)

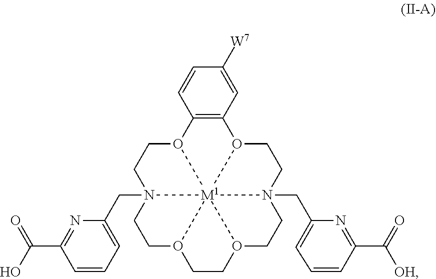

or a pharmaceutically acceptable salt and/or solvate thereof.
M. The compound of any one of Paragraphs J-L, wherein the compound is

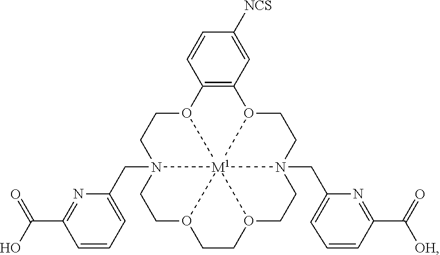

or pharmaceutically acceptable salt and/or solvate thereof.
N. The compound of Paragraph J, wherein the compound of Formula (II) is a compound of Formula (II-B)

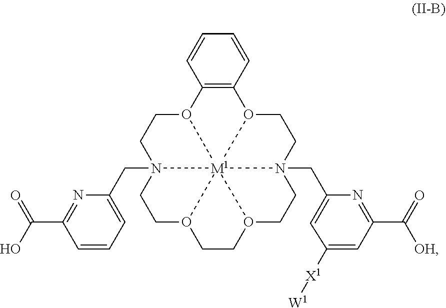

or a pharmaceutically acceptable salt and/or solvate thereof.
O. The compound of Paragraph J, wherein the compound of Formula (II) is a compound of Formula (II-C)

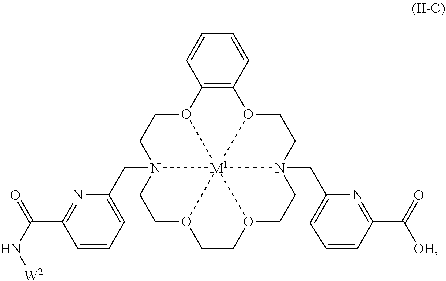

or a pharmaceutically acceptable salt and/or solvate thereof.
P. The compound of Paragraph J, wherein the compound of Formula (II) is a compound of Formula (II-D)

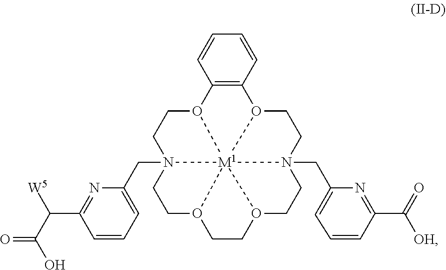

or a pharmaceutically acceptable salt and/or solvate thereof.
Q. The compound of Paragraph J, wherein the compound of Formula (II) is a compound of Formula (II-E)

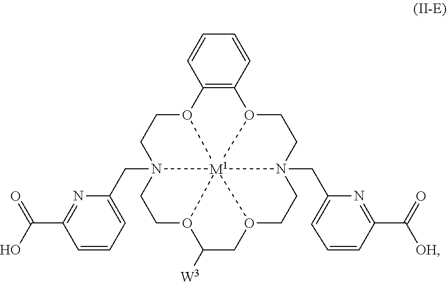

or a pharmaceutically acceptable salt and/or solvate thereof.
R. The compound of Paragraph J, wherein the compound of Formula (II) is a compound of Formula (II-F)

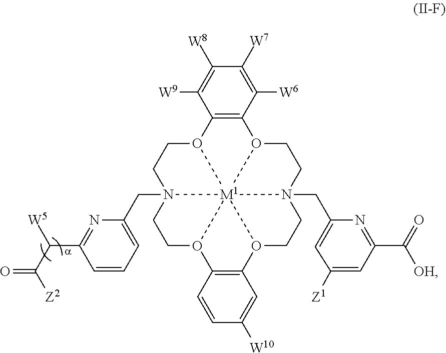

or a pharmaceutically acceptable salt and/or solvate thereof.
S. The compound of any one of Paragraphs J-R, wherein M¹ is independently at each occurrence actinium-225 (²²⁵Ac³⁺), lanthanum-132 (¹³²La³⁺), lanthanum-135 (¹³⁵La³⁺) lutetium-177 (¹⁷⁷Lu³⁺), indium-111 (¹¹¹ In³), radium-223 (²³³Ra²⁺), bismuth-213 (²¹³Bi³⁺) lead-212 (²¹²Pb²⁺ and/or ²¹²Pb⁴⁺), terbium-149 (¹⁴⁹Tb³⁺), fermium-255 (²⁵⁵Fm³⁺) thorium-227 (²²⁷Th⁴⁺), thorium-226 (²²⁶Th⁴⁺), astatine-211 (²¹¹At⁺), astatine-217 (²¹⁷At⁺), uranium-230, scandium-44 (⁴⁴Sc³⁺), scandium-47 (⁴⁷Sc³⁺), gallium-67 (⁶⁷Ga³⁺), or gallium-68 (⁶⁸Ga³⁺
T. The compound of Paragraph S, wherein M¹ is actinium-225 (²²⁵Ac³⁺)
U. A targeting compound of Formula (III)

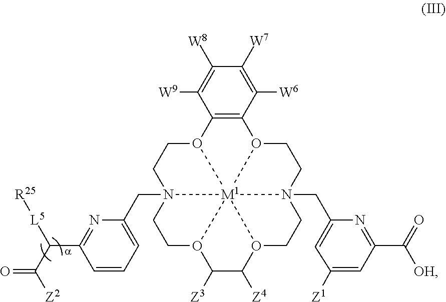

or a pharmaceutically acceptable salt and/or solvate thereof, wherein
M¹ is a radionuclide;
Z¹ is H or —X¹-L¹-R²¹;
Z² is OH or NH-L²-R²²;
Z³ is H or -L³-R²³, and Z⁴ is H or -L⁴-R²⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W⁶, W⁷, W⁸, W⁹, and W¹⁰ are each independently H or -L⁷-R²⁷; L¹, L², L³, L⁴, L⁵, and L⁷ are each independently at each occurrence a bond or a linker group; and
R²¹, R²², R²³, R²⁴, R²⁵, and R²⁷ are each independently comprises an antibody, antibody fragment (e.g., an antigen-binding fragment), a binding moiety, a binding peptide, a binding polypeptide (such as a selective targeting oligopeptide containing up to 50 amino acids), a binding protein, an enzyme, a nucleobase-containing moiety (such as an oligonucleotide, DNA or RNA vector, or aptamer), or a lectin.
V. The targeting compound of Paragraph U, wherein R²¹, R²², R²³, R²⁴, R²⁵, and R²⁷ each independently comprises Codrituzumab (GC33), belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, Etaracizumab, an antigen-binding fragment of any thereof, a prostate specific membrane antigen (“PSMA”) binding peptide, a somatostatin receptor agonist, a bombesin receptor agonist, a seprase binding compound, or a binding fragment of any thereof.
W. The compound of Paragraph U or V, wherein one of W⁶, W⁷, W⁸, and W⁹ is not hydrogen.
X. The targeting compound of any one of Paragraphs U-W, wherein the compound is a compound of Formula (III-A)

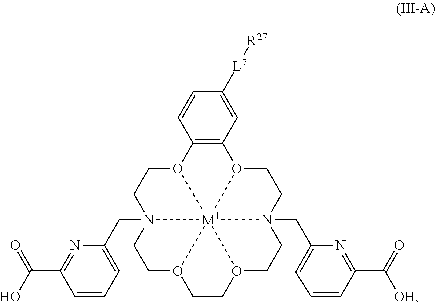

or a pharmaceutically acceptable salt and/or solvate thereof.
Y. The targeting compound of Paragraph U or V, wherein the compound is a compound of Formula (III-B)

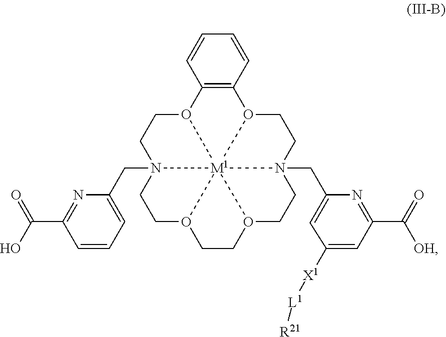

or a pharmaceutically acceptable salt and/or solvate thereof.
Z. The targeting compound of Paragraph U or V, wherein the compound is a compound of Formula (III-C)

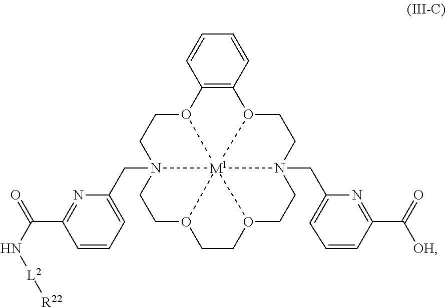

or a pharmaceutically acceptable salt and/or solvate thereof.
AA. The targeting compound of Paragraph U or V, wherein the compound is a compound of Formula (III-D)

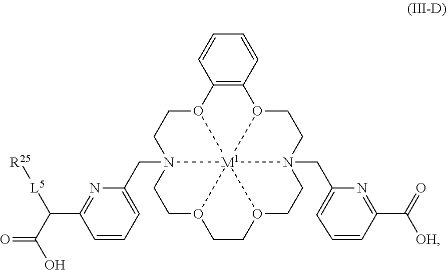

or a pharmaceutically acceptable salt and/or solvate thereof.
BB. The targeting compound of Paragraph U or V, wherein the compound is a compound of Formula (III-E)

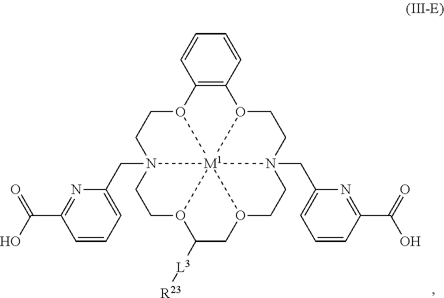

or a pharmaceutically acceptable salt and/or solvate thereof.
CC. The compound of Paragraph U or V, wherein the compound of Formula (III) is a compound of Formula (III-F)

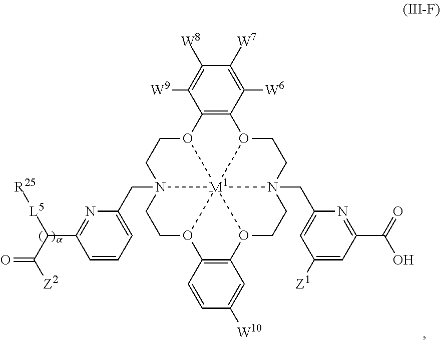

or a pharmaceutically acceptable salt and/or solvate thereof.
DD. The compound of any one of Paragraphs U-CC, wherein M¹ is independently at each occurrence actinium-225 (²²⁵Ac³⁺), lanthanum-132 (¹³²La³⁺), lanthanum-135 (¹³⁵La³⁺) lutetium-177 (¹⁷⁷Lu³⁺), indium-111 (¹¹¹ In³⁺), radium-223 (²³³Ra²⁺), bismuth-213 (²¹³Bi³⁺) lead-212 (²¹²Pb²⁺ and/or ²¹²Pb⁴⁺), terbium-149 (¹⁴⁹Tb³⁺), fermium-255 (²⁵⁵Fm³⁺) thorium-227 (²²⁷Th⁴⁺), thorium-226 (²²⁶Th⁴⁺), astatine-211 (²¹¹At⁺), astatine-217 (²¹⁷At⁺), uranium-230, scandium-44 (⁴⁴Sc³⁺), scandium-47 (⁴⁷Sc³⁺), gallium-67 (⁶⁷Ga³⁺), or gallium-68 (⁶⁸Ga³⁺
EE. The compound of Paragraph DD, wherein M¹ is actinium-225 (²²⁵Ac³⁺)
FF. A modified antibody, modified antibody fragment, or modified binding peptide comprising a linkage arising from conjugation of a compound of Formula (I)

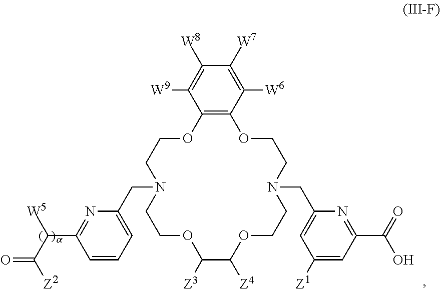

or a pharmaceutically acceptable salt and/or solvate thereof, with an antibody, antibody fragment, or binding peptide, wherein
Z¹ is H or —X¹—W¹;
Z² is OH or NH—W²;
Z³ is H or W³, and Z⁴ is H or W⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W¹, W², W⁶, W⁷, W⁸, and W⁹ are each independently H, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)_{y}—R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group;
W³, W⁴, W⁵, and W¹⁰ are each independently OH, NH₂, SH, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)yₓ-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group; and
R′ is independently at each occurrence H, halo, —N₃, C₁-C₆ alkyl, C₃-C₆ cycloalkyl, C₂-C₆ alkenyl, C₅-C₅ cycloalkenyl, C₂-C₆ alkynyl, C₅-C₁₀ cycloalkynyl, C₅-C₆ aryl, heterocyclyl, or heteroaryl.
GG. The modified antibody, modified antibody fragment, or modified binding peptide of Paragraph FF, wherein the antibody comprises Codrituzumab (GC33), belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, Etaracizumab, a prostate specific membrane antigen (“PSMA”) binding peptide, a somatostatin receptor agonist, a bombesin receptor agonist, or a seprase binding compound.
HH. The modified antibody, modified antibody fragment, or modified binding peptide of Paragraph FF, wherein the antibody fragment comprises an antigen-binding fragment of Codrituzumab (GC33), belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, Etaracizumab, a prostate specific membrane antigen (“PSMA”) binding peptide, a somatostatin receptor agonist, a bombesin receptor agonist, or a seprase binding compound.
II. The modified antibody, modified antibody fragment, or modified binding peptide of any one of Paragraphs FF-HH, wherein the binding peptide comprises Codrituzumab (GC33), or a binding fragment thereof.
JJ. The modified antibody, modified antibody fragment, or modified binding peptide of any one of Paragraphs FF-II, wherein the compound of Formula (I) is a compound of Formula (I-A)

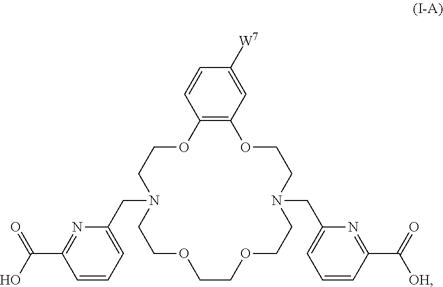

or a pharmaceutically acceptable salt and/or solvate thereof.
KK. The modified antibody, modified antibody fragment, or modified binding peptide of any one of Paragraphs FF-JJ, wherein the linkage is a thiocyante linkage; wherein the thiocyanate linkage arises from conjugation of the compound with the antibody, antibody fragment, or binding peptide; and wherein the compound is

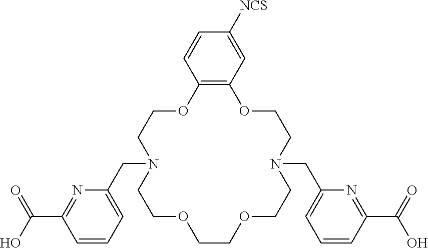

or pharmaceutically acceptable salt and/or solvate thereof
LL. The modified antibody, modified antibody fragment, or modified binding peptide of any one of Paragraphs FF-II, wherein the compound of Formula (I) is a compound of Formula (I-B)

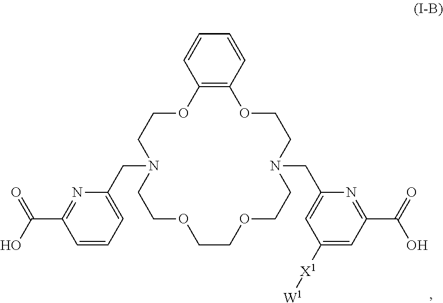

or a pharmaceutically acceptable salt and/or solvate thereof.
MM. The modified antibody, modified antibody fragment, or modified binding peptideof any one of Paragraphs FF-II, wherein the compound of Formula (I) is a compound of Formula (I-C)

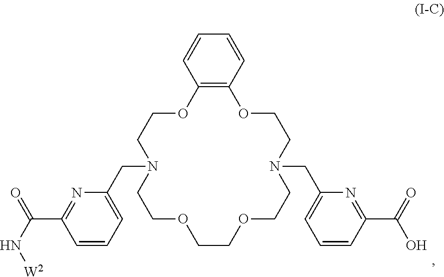

or a pharmaceutically acceptable salt and/or solvate thereof.
NN. The modified antibody, modified antibody fragment, or modified binding peptide of any one of Paragraphs FF-II, wherein the compound of Formula (I) is a compound of Formula (I-D)

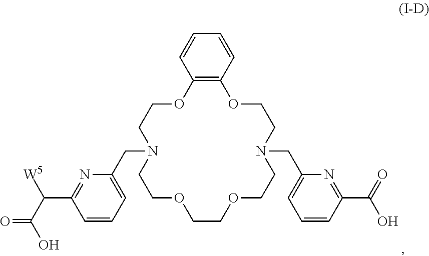

or a pharmaceutically acceptable salt and/or solvate thereof.
OO. The modified antibody, modified antibody fragment, or modified binding peptide of any one of Paragraphs FF-II, wherein the compound of Formula (I) is a compound of Formula (I-E)

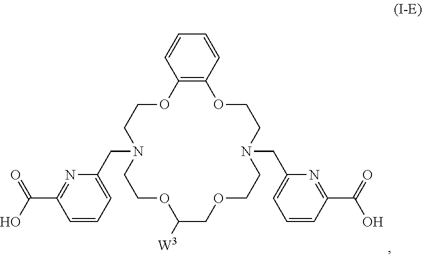

or a pharmaceutically acceptable salt and/or solvate thereof.
PP. The modified antibody, modified antibody fragment, or modified binding peptide of any one of Paragraphs FF-II, wherein the compound of Formula (I) is a compound of Formula (I-F)

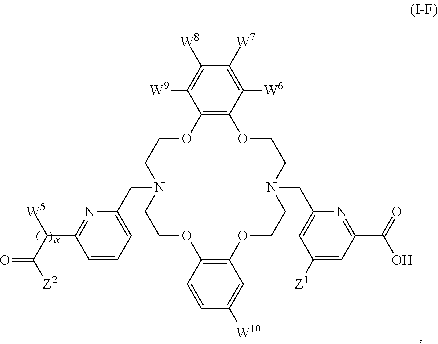

or a pharmaceutically acceptable salt and/or solvate thereof.
QQ. A modified antibody, modified antibody fragment, or modified binding peptide comprising a linkage arising from conjugation of a compound of Formula (II)

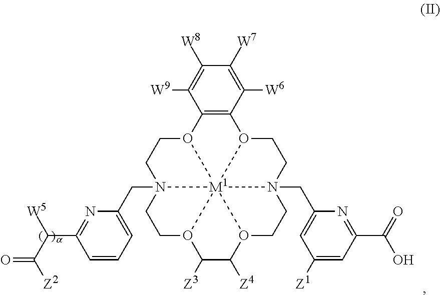

or a pharmaceutically acceptable salt and/or solvate thereof, with an antibody, antibody fragment, or binding peptide, wherein
M¹ is a radionuclide;
Z¹ is H or —X¹—W¹;
Z² is OH or NH—W²;
Z³ is H or W³, and Z⁴ is H or W⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W¹, W², W⁶, W⁷, W⁸, and W⁹ are each independently H, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)_{y}—R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group;
W³, W⁴, W⁵, and W¹⁰ are each independently OH, NH₂, SH, alkyl, cycloalkyl, alkenyl, cycloalkenyl, alkynyl, aryl, heterocyclyl, heteroaryl, —CH₂CH₂—(OCH₂CH₂)_{w}—R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)yₓ-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group; and
R′ is independently at each occurrence H, halo, —N₃, C₁-C₆ alkyl, C₃-C₆ cycloalkyl, C₂-C₆ alkenyl, C₅-C₅ cycloalkenyl, C₂-C₆ alkynyl, C₅-C₁₀ cycloalkynyl, C₅-C₆ aryl, heterocyclyl, or heteroaryl.
RR. The modified antibody, modified antibody fragment, or modified binding peptide of Paragraph QQ, wherein the antibody comprises Codrituzumab (GC33), belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, Etaracizumab, a prostate specific membrane antigen (“PSMA”) binding peptide, a somatostatin receptor agonist, a bombesin receptor agonist, or a seprase binding compound.
SS. The modified antibody, modified antibody fragment, or modified binding peptide of any one of Paragraph QQ, wherein the antibody fragment comprises an antigen-binding fragment of Codrituzumab (GC33), belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, Etaracizumab, a prostate specific membrane antigen (“PSMA”) binding peptide, a somatostatin receptor agonist, a bombesin receptor agonist, or a seprase binding compound.
TT. The modified antibody, modified antibody fragment, or modified binding peptide of any one of Paragraphs QQ-SS, wherein the binding peptide comprises Codrituzumab (GC33), or a binding fragment thereof.
UU. The modified antibody, modified antibody fragment, or modified binding peptide of any one of Paragraphs QQ-TT, wherein the compound of Formula (II) is a compound of Formula (II-A)

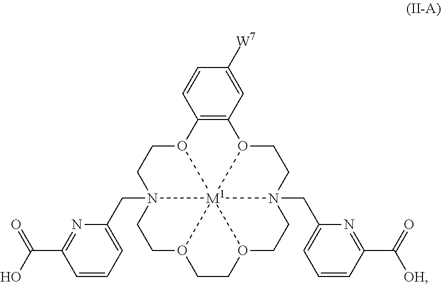

or a pharmaceutically acceptable salt and/or solvate thereof.
VV. The modified antibody, modified antibody fragment, or modified binding peptide of Paragraph UU, wherein the linkage is a thiocyante linkage; wherein the thiocyanate linkage arises from conjugation of the compound with the antibody, antibody fragment, or binding peptide; and wherein the compound is

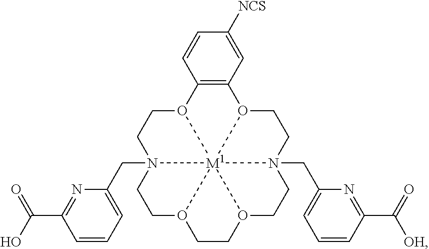

or a pharmaceutically acceptable salt and/or solvate thereof.
WW. The modified antibody, modified antibody fragment, or modified binding peptide of any one of Paragraphs QQ-TT, wherein the compound of Formula (II) is a compound of Formula (II-B)

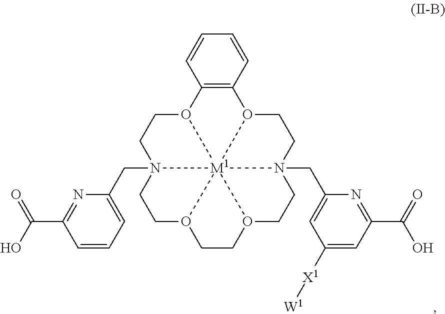

or a pharmaceutically acceptable salt and/or solvate thereof.
XX. The modified antibody, modified antibody fragment, or modified binding peptide of any one of Paragraphs QQ-TT, wherein the compound of Formula (II) is a compound of Formula (II-C)

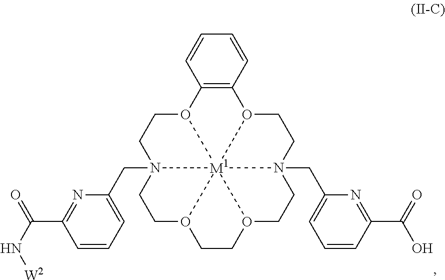

or a pharmaceutically acceptable salt and/or solvate thereof.
YY. The modified antibody, modified antibody fragment, or modified binding peptide of any one of Paragraphs QQ-TT, wherein the compound of Formula (II) is a compound of Formula (II-D)

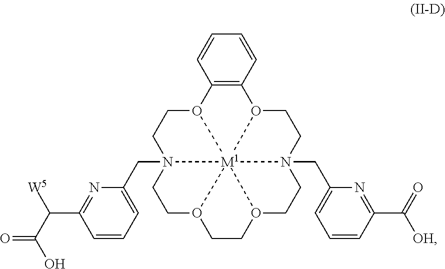

or a pharmaceutically acceptable salt and/or solvate thereof.
ZZ. The modified antibody, modified antibody fragment, or modified binding peptide of any one of Paragraphs QQ-TT, wherein the compound of Formula (II) is a compound of Formula (II-E)

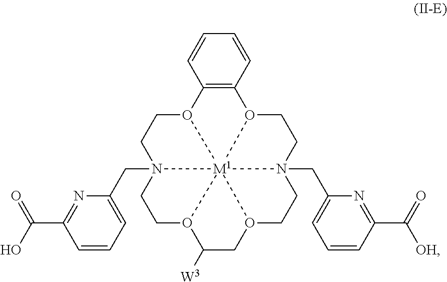

or a pharmaceutically acceptable salt and/or solvate thereof.
AAA. The modified antibody, modified antibody fragment, or modified binding peptide of any one of Paragraphs QQ-TT, wherein the compound of Formula (II) is a compound of Formula (II-F)

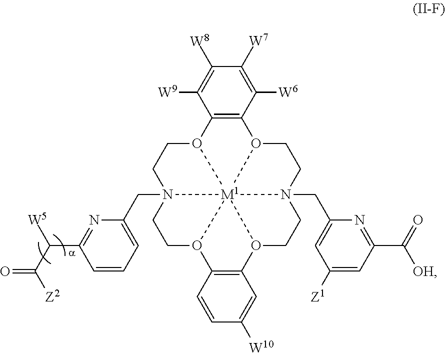

or a pharmaceutically acceptable salt and/or solvate thereof.
BBB. The modified antibody, modified antibody fragment, or modified binding peptide of any one of Paragraphs QQ-AAA, wherein M¹ is independently at each occurrence actinium-225 (²²⁵Ac³⁺), lanthanum-132 (¹³²La³⁺), lanthanum-135 (¹³⁵La³⁺), lutetium-177 (¹⁷⁷Lu³⁺), indium-111 (¹¹¹In³⁺), radium-223 (²³³Ra²⁺), bismuth-213 (²¹³Bi³⁺), lead-212 (²¹²Pb²⁺ and/or ²¹²Pb⁴⁺), terbium-149 (¹⁴⁹Tb³⁺), fermium-255 (²⁵⁵Fm³⁺), thorium-227 (²²⁷Th⁴⁺), thorium-226 (²²⁶Th⁴⁺), astatine-211 (²¹¹At⁺), astatine-217 (²¹⁷At⁺), uranium-230, scandium-44 (⁴⁴Sc³⁺), scandium-47 (⁴⁷Sc³⁺), gallium-67 (⁶⁷Ga³⁺), or gallium-68 (⁶⁸Ga³⁺).
CCC. The modified antibody, modified antibody fragment, or modified binding peptide of Paragraph BBB, wherein M¹ is actinium-225 (²²⁵Ac³⁺)
DDD. A composition comprising a pharmaceutically acceptable carrier and a compound of any one of Paragraphs A-T.
EEE. A composition comprising a pharmaceutically acceptable carrier and a targeting compound of any one of Paragraphs U-EE or comprising a pharmaceutically acceptable carrier and a modified antibody, modified antibody fragment, or modified binding peptide of any one of Paragraphs FF-CCC.
FFF. A pharmaceutical composition useful in targeted radiotherapy in a subject of cancer and/or mammalian tissue overexpressing glypican-3 (GPC3) receptor and/or a mammalian tissue overexpressing PSMA, wherein the pharmaceutical composition comprises a pharmaceutically acceptable carrier and a compound of any one of Paragraphs U-EE or a modified antibody, modified antibody fragment, or modified binding peptide of any one of Paragraphs FF-CCC.
GGG. The pharmaceutical composition of Paragraph FFF, wherein the pharmaceutical composition comprises an effective amount for treating the cancer and/or mammalian tissue overexpressing glypican-3 (GPC3) receptor and/or mammalian tissue overexpressing PSMA of the compound or an effective amount for treating the cancer and/or mammalian tissue overexpressing glypican-3 (GPC3) receptor and/or mammalian tissue overexpressing PSMA of the modified antibody, modified antibody fragment, or modified binding peptide.
HHH. The pharmaceutical composition of Paragraph FFF or Paragraph GGG, where the subject suffers from a mammalian tissue overexpressing a glypican-3 (GPC3) receptor and/or a mammalian tissue overexpressing PSMA
III. The pharmaceutical composition of any one of Paragraphs FFF-HHH, wherein the subject suffers from one or more of, a growth hormone producing tumor, a neuroendocrine tumor, a pituitary tumor, a vasoactive intestinal peptide-secreting tumor, a small cell carcinoma of the lung, gastric cancer tissue, pancreatic cancer tissue, and a neuroblastoma.
JJJ. The pharmaceutical composition of any one of Paragraphs FFF-III, wherein the subject suffers from one or more of a liver cancer, a glioma, a breast cancer, an adrenal cortical cancer, a cervical carcinoma, a vulvar carcinoma, an endometrial carcinoma, a primary ovarian carcinoma, a metastatic ovarian carcinoma, a non-small cell lung cancer, a small cell lung cancer, a bladder cancer, a colon cancer, a primary gastric adenocarcinoma, a primary colorectal adenocarcinoma, a renal cell carcinoma, and a prostate cancer.
KKK. The pharmaceutical composition of any one of Paragraphs FFF-JJJ, wherein the subject suffers from liver cancer.
LLL. The pharmaceutical composition of any one of Paragraphs FFF-KKK, wherein the pharmaceutical composition is formulated for intraveneous administration, optionally comprising sterilized water, Ringer's solution, or an isotonic aqueous saline solution.
MMM. The pharmaceutical composition of any one of FFF-LLL, wherein the effective amount of the compound is from about 0.01 μg to about 10 mg of the compound per gram of the pharmaceutical composition.
NNN. The pharmaceutical composition of any one of Paragraphs FFF-MMM, wherein the pharmaceutical composition is provided in an injectable dosage form.
OOO. A method of treating a subject, wherein the method comprises administering a targeting compound of any one of Paragraphs U-EE to the subject or administering a modified antibody, modified antibody fragment, or modified binding peptide of any one of Paragraphs FF-CCC.
PPP. The method of Paragraph 000, wherein the subject suffers from cancer and/or mammalian tissue overexpressing a glypican-3 (GPC3) receptor and/or a mammalian tissue overexpressing PSMA.
QQQ. The method of Paragraph 000, wherein the method comprises administering an effective amount for treating the cancer and/or mammalian tissue overexpressing a glypican-3 (GPC3) receptor and/or mammalian tissue overexpressing PSMA of the compound or an effective amount for treating the cancer and/or mammalian tissue overexpressing a glypican-3 (GPC3) receptor and/or mammalian tissue overexpressing PSMA of the modified antibody, modified antibody fragment, or modified binding peptide.
RRR. The method of any one of Paragraphs 000-QQQ, wherein the subject suffers from one or more of a liver cancer, a glioma, a breast cancer, an adrenal cortical cancer, a cervical carcinoma, a vulvar carcinoma, an endometrial carcinoma, a primary ovarian carcinoma, a metastatic ovarian carcinoma, a non-small cell lung cancer, a small cell lung cancer, a bladder cancer, a colon cancer, a primary gastric adenocarcinoma, a primary colorectal adenocarcinoma, a renal cell carcinoma, and a prostate cancer.
SSS. The method of any one of Paragraphs 000-RRR, wherein the administering comprises parenteral administration.
TTT. The method of any one of Paragraphs 000-SSS, wherein the administering comprises intraveneous administration.
UUU. The method of any one of Paragraphs 000-TTT, wherein the effective amount is from about 0.1 μg to about 50 μg per kilogram of subject mass.

Other embodiments are set forth in the following claims, along with the full scope of equivalents to which such claims are entitled.

1. A compound of Formula (I) 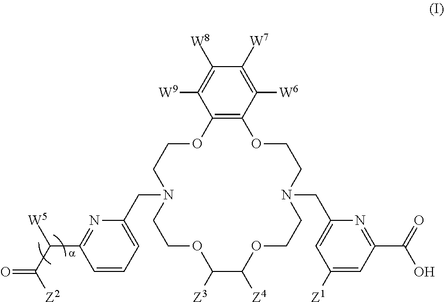
or a pharmaceutically acceptable salt and/or solvate thereof, wherein
Z¹ is H or —X¹—W¹;
Z² is OH or NH—W²;
Z³ is H or W³, and Z⁴ is H or W⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W¹, W², W⁶, W⁷, W⁸, and W⁹ are each independently H, alkyl, cycloalkyl, alkenyl, cycloalkenyl, alkynyl, aryl, heterocyclyl, heteroaryl, —CH₂CH₂—(OCH₂CH₂), —R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)y-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group;
W³, W⁴, W⁵, and W¹⁰ are each independently OH, NH₂, SH, alkyl, cycloalkyl, alkenyl, cycloalkenyl, alkynyl, aryl, heterocyclyl, heteroaryl, —CH₂CH₂—(OCH₂CH₂), —R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)yₓ-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group; and
R′ is independently at each occurrence H, halo, —N₃, C₁-C₆ alkyl, C₃-C₆ cycloalkyl, C₂-C₆ alkenyl, C₅-C₈ cycloalkenyl, C₂-C₆ alkynyl, C₈-C₁₀ cycloalkynyl, C₅-C₆ aryl, heterocyclyl, or heteroaryl.

2. The compound of claim 1, wherein at least one of W⁶, W⁷, W⁸, and W⁹ is not hydrogen.

3. The compound of claim 1, wherein the compound is a compound of Formula (I-A) 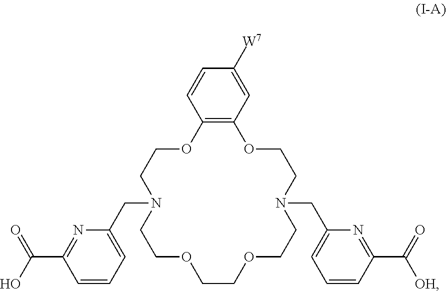
or a pharmaceutically acceptable salt and/or solvate thereof.

4. The compound of claim 1, wherein the compound is 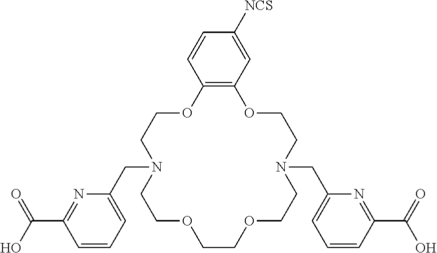
or pharmaceutically acceptable salt and/or solvate thereof.

5. The compound of claim 1, wherein the compound of Formula (I) is a compound of Formula (I-B) 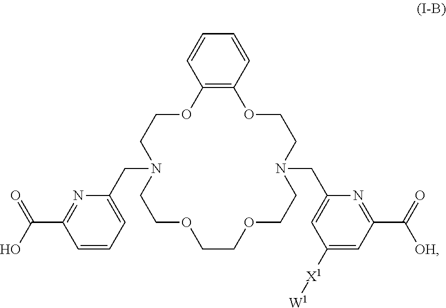
or a pharmaceutically acceptable salt and/or solvate thereof.

6. The compound of claim 1, wherein the compound of Formula (I) is a compound of Formula (I-C) 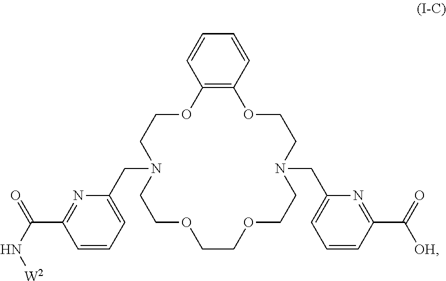
or a pharmaceutically acceptable salt and/or solvate thereof.

7. The compound of claim 1, wherein the compound of Formula (I) is a compound of Formula (I-D) 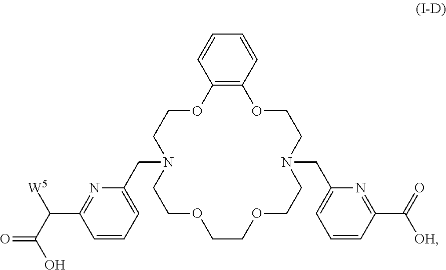
or a pharmaceutically acceptable salt and/or solvate thereof.

8. The compound of claim 1, wherein the compound is a compound of Formula (I-E) 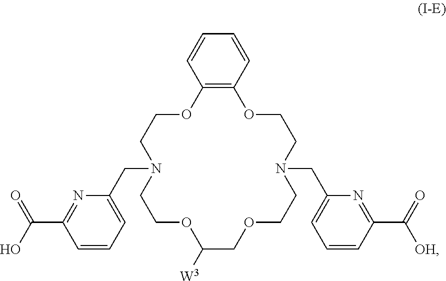
or a pharmaceutically acceptable salt and/or solvate thereof.

9. The compound of claim 1, wherein the compound is a compound of Formula (I-F) 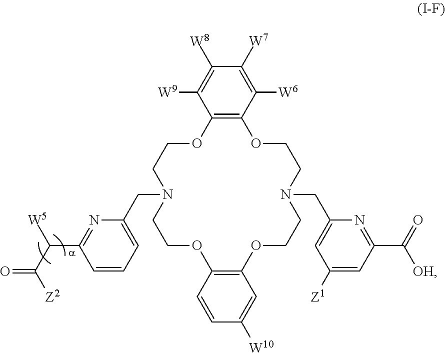
or a pharmaceutically acceptable salt and/or solvate thereof.

10. A compound of Formula (II) 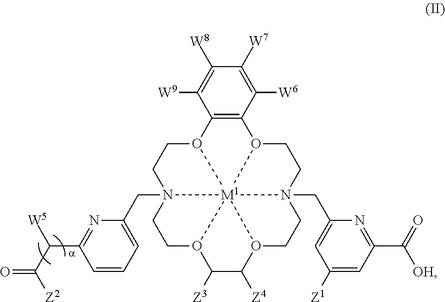
or a pharmaceutically acceptable salt and/or solvate thereof, wherein
M¹ is a radionuclide;
Z¹ is H or —X¹—W¹;
Z² is OH or NH—W²;
Z³ is H or W³, and Z⁴ is H or W⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W¹, W², W⁶, W⁷, W⁸, and W⁹ are each independently H, alkyl, cycloalkyl, alkenyl, cycloalkenyl, alkynyl, aryl, heterocyclyl, heteroaryl, —CH₂CH₂—(OCH₂CH₂), —R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)y-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group;
W³, W⁴, W⁵, and W¹⁰ are each independently OH, NH₂, SH, alkyl, cycloalkyl, alkenyl, cycloalkenyl, alkynyl, aryl, heterocyclyl, heteroaryl, —CH₂CH₂—(OCH₂CH₂), —R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)yₓ-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group; and
R′ is independently at each occurrence H, halo, —N₃, C₁-C₆ alkyl, C₃-C₆ cycloalkyl, C₂-C₆ alkenyl, C₅-C₈ cycloalkenyl, C₂-C₆ alkynyl, C₈-C₁₀ cycloalkynyl, C₅-C₆ aryl, heterocyclyl, or heteroaryl.

11. (canceled)

12. (canceled)

13. (canceled)

14. (canceled)

15. (canceled)

16. (canceled)

17. (canceled)

18. (canceled)

19. (canceled)

20. (canceled)

21. A targeting compound of Formula (III) 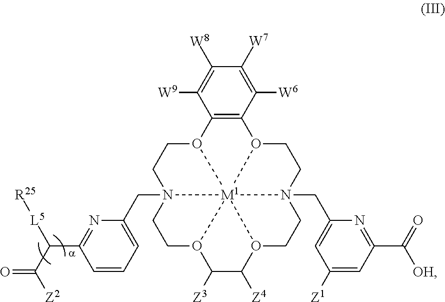
or a pharmaceutically acceptable salt and/or solvate thereof, wherein
M¹ is a radionuclide;
Z¹ is H or —X¹-L¹-R²¹;
Z² is OH or NH-L²-R²²;
Z³ is H or -L³-R²³, and Z⁴ is H or -L⁴-R²⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W⁶, W⁷, W⁸, W⁹, and W¹⁰ are each independently H or -L⁷-R²⁷;
L¹, L², L³, L⁴, L⁵, and L⁷ are each independently at each occurrence a bond or a linker group; and
R²¹, R²², R²³, R²⁴, R²⁵, and R²⁷ are each independently comprises an antibody, antibody fragment (e.g., an antigen-binding fragment), a binding moiety, a binding peptide, a binding polypeptide (such as a selective targeting oligopeptide containing up to 50 amino acids), a binding protein, an enzyme, a nucleobase-containing moiety (such as an oligonucleotide, DNA or RNA vector, or aptamer), or a lectin.

22. The targeting compound of claim 21, wherein R²¹, R²², R²³, R²⁴, R²⁵, and R²⁷ each independently comprises Codrituzumab (GC33), belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, Etaracizumab, an antigen-binding fragment of any thereof, a prostate specific membrane antigen (“PSMA”) binding peptide, a somatostatin receptor agonist, a bombesin receptor agonist, a seprase binding compound, or a binding fragment of any thereof.

23. (canceled)

24. (canceled)

25. (canceled)

26. (canceled)

27. (canceled)

28. (canceled)

29. (canceled)

30. (canceled)

31. (canceled)

32. A modified antibody, modified antibody fragment, or modified binding peptide comprising a linkage arising from conjugation of a compound of Formula (I) 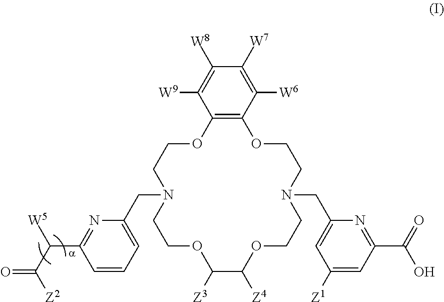
or a pharmaceutically acceptable salt and/or solvate thereof, with an antibody, antibody fragment, or binding peptide, wherein
Z¹ is H or —X¹—W¹;
Z² is OH or NH—W²;
Z³ is H or W³, and Z⁴ is H or W⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W¹, W², W⁶, W⁷, W⁸, and W⁹ are each independently H, alkyl, cycloalkyl, alkenyl, cycloalkenyl, alkynyl, aryl, heterocyclyl, heteroaryl, —CH₂CH₂—(OCH₂CH₂), —R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)y-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group;
W³, W⁴, W⁵, and W¹⁰ are each independently OH, NH₂, SH, alkyl, cycloalkyl, alkenyl, cycloalkenyl, alkynyl, aryl, heterocyclyl, heteroaryl, —CH₂CH₂—(OCH₂CH₂), —R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)yₓ-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group; and
R′ is independently at each occurrence H, halo, —N₃, C₁-C₆ alkyl, C₃-C₆ cycloalkyl, C₂-C₆ alkenyl, C₅-C₈ cycloalkenyl, C₂-C₆ alkynyl, C₈-C₁₀ cycloalkynyl, C₅-C₆ aryl, heterocyclyl, or heteroaryl.

33. (canceled)

34. (canceled)

35. (canceled)

36. (canceled)

37. (canceled)

38. (canceled)

39. (canceled)

40. (canceled)

41. (canceled)

42. (canceled)

43. A modified antibody, modified antibody fragment, or modified binding peptide comprising a linkage arising from conjugation of a compound of Formula (II) 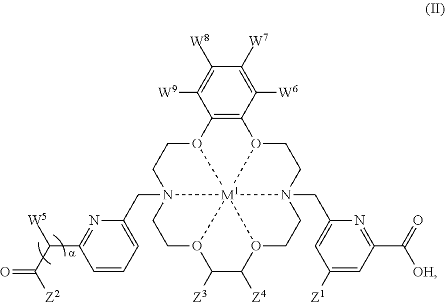
or a pharmaceutically acceptable salt and/or solvate thereof, with an antibody, antibody fragment, or binding peptide, wherein
M¹ is a radionuclide;
Z¹ is H or —X¹—W¹;
Z² is OH or NH—W²;
Z³ is H or W³, and Z⁴ is H or W⁴; or Z³ and Z⁴ taken together with the carbon atoms to which they are bound are a 6-membered aryl ring optionally substituted by W¹⁰;
α is 0 or 1;
X¹ is O, NH, S, or a covalent bond;
W¹, W², W⁶, W⁷, W⁸, and W⁹ are each independently H, alkyl, cycloalkyl, alkenyl, cycloalkenyl, alkynyl, aryl, heterocyclyl, heteroaryl, —CH₂CH₂—(OCH₂CH₂), —R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)y-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group;
W³, W⁴, W⁵, and W¹⁰ are each independently OH, NH₂, SH, alkyl, cycloalkyl, alkenyl, cycloalkenyl, alkynyl, aryl, heterocyclyl, heteroaryl, —CH₂CH₂—(OCH₂CH₂), —R′ where w is 1, 2, 3, 4, 5, 6, 7, 8, 9, or 10, or —CH₂CH₂—(OCH₂CH₂)ₓ—OR′ where x is 1, 2, 3, 4, 5, 6, 7, 8, 9, or 10, each of which may optionally be substituted with one or more of halo, —N₃, —OR′, —CH₂CH₂—(OCH₂CH₂)yₓ-R′ where y is 1, 2, 3, 4, 5, 6, 7, 8, 9, or 10, —CH₂CH₂—(OCH₂CH₂)_{z}—OR′ where z is 1, 2, 3, 4, 5, 6, 7, 8, 9, or 10, —SR′, —OC(O)R′, —C(O)OR′, —C(S)OR′, —S(O)R′, —SO₂R′, —SO₂(OR′), —SO₂NR′₂, —P(O)(OR′)₂, —P(O)R′(OR′), —P(O)R′₂, —CN, —OCN, —SCN, —NCO, —NCS, —NR′—NH₂, —N═C═N—R′, —SO₂Cl, —C(O)Cl, or an epoxide group; and
R′ is independently at each occurrence H, halo, —N₃, C₁-C₆ alkyl, C₃-C₆ cycloalkyl, C₂-C₆ alkenyl, C₅-C₈ cycloalkenyl, C₂-C₆ alkynyl, C₈-C₁₀ cycloalkynyl, C₅-C₆ aryl, heterocyclyl, or heteroaryl.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. A composition comprising a pharmaceutically acceptable carrier and a compound of claim 1.

57. A composition comprising a pharmaceutically acceptable carrier and a targeting compound of claim 21.

58. A pharmaceutical composition useful in a subject in targeted radiotherapy of cancer and/or a mammalian tissue overexpressing glypican-3 (GPC3) receptor and/or a mammalian tissue overexpressing prostate specific membrane antigen (PSMA), wherein the pharmaceutical composition comprises a pharmaceutically acceptable carrier and a compound of claim 21.

59. (canceled)

60. (canceled)

61. (canceled)

62. (canceled)

63. (canceled)

64. (canceled)

65. (canceled)

66. (canceled)

67. A method of treating a subject, wherein the method comprises administering a targeting compound of claim 21 to the subject.

68. A pharmaceutical composition useful in a subject in targeted radiotherapy of cancer and/or a mammalian tissue overexpressing glypican-3 (GPC3) receptor and/or a mammalian tissue overexpressing prostate specific membrane antigen (PSMA), wherein the pharmaceutical composition comprises a pharmaceutically acceptable carrier and a compound of claim 32.

69. A method of treating a subject, wherein the method comprises administering a modified antibody, modified antibody fragment, or modified binding peptide of claim 32 to the subject.
